(19) United States
(12) Patent Application Publication
(10) Pub. No.: US 2010/0273151 A1
(43) Pub. Date: Oct. 28, 2010

(54) GENOME-WIDE ANALYSIS OF PALINDROME FORMATION AND DNA METHYLATION

(75) Inventors: Stephen J. Tapscott, Seattle, WA (US); Hisashi Tanaka, Pepper Pike, OH (US); Meng-Chao Yao, Taipei (TW); Scott J. Diede, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fred Hutchinson Cancer Research Center, Seattle, WA (US)

(21) Appl. No.: 12/472,311

(22) Filed: May 26, 2009

Related U.S. Application Data
(63) Continuation-in-part of application No. 11/142,091, filed on May 31, 2005, now abandoned.
(60) Provisional application No. 60/575,331, filed on May 28, 2004.

(51) Int. Cl. C12Q 1/68

(52) U.S. Cl. 435/6

(57) ABSTRACT

The present disclosure provides methods for detecting the genome-wide presence of methylated DNA and palindrome formation. The present disclosure also provides methods for specific enrichment of methylated DNA or DNA having a DNA palindrome. These methods have demonstrated that somatic palindromes and methylated DNA occur frequently and are widespread in human cancers. Individual tumor types have a characteristic non-random distribution of palindromes in their genome and a small subset of the palindromic loci are associate with gene amplification. The disclosed method can be used to define the plurality of genomic DNA palindromes and regions having methylated DNA associated with various tumor types and can provide methods for the classification of tumors, and the diagnosis, early detection of cancer as well as the monitoring of disease recurrence and assessment of residual disease.

### CROSS-REFERENCES TO RELATED APPLICATIONS

The present application is a continuation-in-part of U.S. patent application Ser. No. 11/142,091, which claims priority to U.S. Provisional Patent Application No. 60/575,331, filed May 28, 2004, the entire disclosures of which are incorporated by reference herein.

### STATEMENT AS TO RIGHTS TO INVENTIONS MADE UNDER FEDERALLY SPONSORED RESEARCH OR DEVELOPMENT

This invention was made with government support under Grant Nos. R01AR 045113, R01GM 26210, K12 HD43376 and 2T32CA009351 awarded by the National Institutes of Health. The Government has certain rights in the invention.

### BACKGROUND

Cancer is a disease of impaired genetic integrity. In most cases disturbed genetic integrity is observed at the chromosome level and include a configuration called anaphase bridges, which are most likely derived from dicentric or ring chromosomes segregating into two different daughter cells in the process of the breakage-fusion-bridge (BFB) cycle. The BFB cycles have been shown to generate large DNA palindromes with structural gains and losses at the termini of sister chromatids by creating recombinogenic free ends, followed by sister chromatid fusions at each cycle. Evidence has been accumulating that the BFB cycle is a major driving force for genetic diversity generating chromosome aberrations in cancer cells. Telomere shortening in mice lacking the Telomerase RNA component (TR) results in chromosome end-to-end fusions that are enhanced by p53 deficiency. Initiation of neoplastic lesions and frequent anaphase bridges are both increased with progressive telomere shortening in mouse intestinal tumors, and human colon carcinomas show a sharp increase of anaphase bridges at the early stage of carcinogenesis. This suggests that telomere dysfunction can generate dicentric chromosomes by end-to-end fusions and trigger the BFB cycle, providing genetic heterogeneity that furthers the malignant phenotype. Spontaneous and/or ionizing radiation induced chromosome end-to-end fusions are also seen in cells that have cancer-predisposing mutations, such as a deficiency in the DNA damage checkpoint function (ATM) (Metcalf et al. Nat. Genet. 13:350-353 (1996)), non-homologous end-to-end joining (NHEJ) repair of DNA double strand breaks (DSB) (DNA-PKcs, Ku70, Ku80, Lig4, XRCC4) (Bailey et al., Proc. Natl. Acad. Sci. USA 96:14899-14904 (1999); Ferguson et al., Proc. Natl. Acad. Sci. USA 97: 6630-6633 (2000); Gao et al., Nature 404:897-900 (2000); Hsu et al., Genes Dev. 14:2807-2812 (2000)), RAD51D (Tarsounas et al., Cell 117:337-347 (2004)) and histone H2AX (Bassing et al., Proc. Natl. Acad. Sci. USA 99:8173-8178 (2002)). Moreover in mice deficient in both p. 53 and NHEJ, co-amplification of c-myc and IgH in pro B cell lymphomas is initiated by the BFB cycle after RAG-induced DSB at the IgH locus is incorrectly repaired by fusion to the c-myc gene to form a dicentric chromosome (Gao et al., supra. (2000); Zhu et al., Cell 109: 811-821 (2002)). This indicates that improper DSB repair also could trigger the BFB cycle for further chromosome aberrations.

The BFB cycle has also been implicated as a common mechanism for intrachromosomal gene amplification (Coquelle et al., Cell 89:215-225 (1997); Ma et al., Genes Dev. 7:605-620 (1993); Smith et al., Proc. Natl. Acad. Sci. USA 89:5427-5431 (1992); Toledo et al., EMBO J. 11:2665-2673 (1992)). Studies of gene amplifications selected by drug resistance in rodent cells have shown that most of the amplifications are associated with large DNA palindromes (Coquelle et al., supra. (1997); Ma et al., supra. (1993); Ruiz and Wahl, Mol. Cell. Biol. 8:4302-4313 (1988); Smith et al., Proc. Natl. Acad. Sci. USA 89:5427-5431 (1992); Toledo et al., supra. (1992)). An initial palindromic duplication of the dhfr gene induced by I-SceI-induced chromosomal DSB triggers BFB cycles and results in further dhfr amplification, where the initial formation of a palindrome appears to be the rate-limiting step for subsequent gene amplification (Tanaka et al., Proc. Natl. Acad. Sci. USA 99:8772-8777 (2002)). Various clastogenic drugs induce initial chromosome breaks at the common loci that bracket the palindromic amplification of the selected gene (Coquelle et al., supra. (1997)), suggesting the presence of specific loci in the genome susceptible to palindrome formation.

Although cytogenetic studies of cancer cells also indicate that oncogene amplifications occur as large DNA palindromes by BFB cycles (Ciullo et al., Hum. Mol. Genet. 11:2887-2894 (2002); Hellman et al., Cancer Cell 1:89-97 (2002)), little is known about how prevalent this type of chromosome aberration is in cancer cells. Given the fact that telomere dysfunction and impaired DNA damage checkpoint/repair functions can trigger BFB cycles and are major causes of chromosome instability, somatic palindrome formation might be widespread in cancer cells and provide a platform for additional gene amplification. However, our molecular analysis of the structure of amplified loci in cancer cells has been limited by the fact that the duplication covers very large regions of the chromosome.

DNA methylation in vertebrates is a well-established epigenetic mechanism that controls a variety of important developmental functions including X chromosome inactivation, genomic imprinting and transcriptional regulation. Cytosine DNA methylation in mammals predominantly occurs at CpG dinucleotides, of which more than 70% are methylated. CpG islands are clusters of CpG dinucleotides that mostly remain unmethylated and could play an important role in gene regulation. There are approximately 27,000 and 15,500 CpG islands in the human and mouse genomes respectively, among which 10,000 are highly conserved between these two organisms. CpG islands often reside in 5′ regulatory regions and exons of genes (promoter CpG islands), and recent computational analysis indicates that a significant proportion of CpG islands are in other exons and intergenic regions. Although CpG islands are generally considered to be unmethylated, a significant fraction of them can be methylated. For example, a number of studies have shown that differential methylation of promoter CpG islands leads to transcriptional repression of tumor suppressor genes in cancer cells. There also are a few CpG islands that undergo tissue specific methylation during development. However, these examples are limited in number and fail to reveal the full scope of dynamic changes in methylation status. For instance, there is general hypomethylation in cancer cells, and a genome-wide demethylation-remethylation transition occurs during normal development.

Currently, a number of genome-wide methods to determine DNA methylation states have been reported (Suzuki & Bird, Nat. Rev. Genet., 9:465-476 (2008)). Certain methods, such as Comprehensive High-Throughput Arrays for Relative Methylation (CHARM) (Irizarry et al., Genome Research 18:780-790 (2008)) and HpaII-tiny fragment Enrichment by Ligation-mediated PCR (HELP) (Khulan et al., Genome Research 16:1046-1055 (2006)), use restriction enzymes that are either sensitive, insensitive, or specific or CpG methylation to interrogate DNA methylation states. These methods can be disadvantageous because each method is dependent on the presence and optimal spacing of methylation sensitive restriction enzyme recognition sites and variable methylation patterns with similar densities can cause differential signals. Other methods are based on affinity purification of methylated DNA. One commonly used method is methylated DNA immunoprecipitation (MeDIP) (Weber et al., Nat. Genet., 37:853-862 (2005)), which uses an antibody to 5-methylcytosine to assess DNA methylation. Another set of techniques utilizes a methyl-CpG binding protein to enrich for DNA methylation. Two such techniques have been described, one using the rat MeCP2 protein (Cross et al., Nat. Genet. 6:236-244 (1994)) and another using the MBD2/MBD3L1 complex (Rauch et al., Cancer Research 66:7939-7947 (2006)). All of these techniques to assess genome-wide methylation patterns can use a variety of microarray platforms to generate ‘methylome’ datasets.

The present disclosure provides methods for the study of the genome-wide distribution of somatic palindrome formation and methylated DNA.

### BRIEF SUMMARY

Genome-wide methods for analyzing palindrome formation and DNA methylation are disclosed. In certain embodiments, the methods generally include isolating genomic DNA including a DNA palindrome and a methylated DNA, fragmenting the genomic DNA, denaturing unmethylated genomic DNA, rehybridizing the denatured unmethylated DNA under suitable conditions for the DNA palindrome to form a snap back DNA, digesting the rehybridized DNA with a nuclease that digests single strand DNA, and identifying the genomic DNA including the methylated DNA and the snap back DNA including the DNA palindrome. The methods can further include identifying regions of the genomic DNA including the methylated DNA and the DNA palindrome by hybridization of the genomic DNA fragments with a human genomic DNA array.

In one embodiment, the method includes the steps of: a) isolating genomic DNA including the DNA palindrome or the methylated DNA from a population of cells; b) denaturing the isolated, unmethylated DNA; c) rehybridizing the denatured isolated DNA under suitable conditions for the DNA palindrome to form a snap back DNA and to keep the methylated DNA hybridized; d) digesting the rehybridized DNA with a nuclease that digests single strand DNA to form double stranded DNA fragments including the snap back DNA and the methylated DNA; e) digesting the double stranded DNA fragments including the snap back DNA with a nucleotide sequence specific restriction enzyme; f) adding a sequence specific linker nucleotide sequence to one end of each stand of the double strand DNA including the snap back DNA; g) amplifying the DNA fragments including the added linker using a labeled linker sequence specific primer corresponding to the sequence specific linker added in step (f); and h) hybridizing the methylated DNA and the amplified DNA fragments including the snap back DNA to a genomic DNA library and identifying the genomic DNA region including the palindrome or the methylated DNA.

The method can further include steps wherein the amplified DNA fragments include the snap back DNA are mixed and co-hybridized in step (h) with a sample of high molecular weight DNA from a normal cell population that has been digested with S1 nuclease, and the restriction enzyme of step (e), adding a linker labeled with a second single label, and amplified. As with the snap back DNA sample, the normal high molecular weight DNA will have been digested with S1 nuclease and with the same restriction enzymes of step (e) as the snap back DNA sample, have the sequence specific linker added and the DNA fragments amplified and labeled using a sequence-specific primer corresponding to the sequence specific linker added in the previous step which contains a second label, prior to mixing with the snap back DNA and co-hybridization.

Any single strand nuclease can be used in the present methods including, for example S1 nuclease. Further, the genomic DNA fragments can be digested with any restriction enzyme that specifically cuts double stranded DNA. Typically, the DNA will be digested with two or more restriction enzymes and the profiles compared. In one embodiment of the present disclosure the DNA is digested separately with MspI, TaqI, or MseI. To prepare the high molecular weight genomic DNA, total DNA from a sample of a cell population is isolated and the isolated genomic DNA is fragmented by a chemical, physical, or enzymatic method. In one embodiment the genomic DNA is digested with, for example, SalI, but any other restriction enzyme that results in high molecular weight DNA can also be used.

The present disclosure also provides methods for classifying a population of cancer cells. The methods can include identifying regions of genomic DNA including a methylated DNA and a snap back DNA having a DNA palindrome, and using the identity of genomic DNA regions including fragmenting the genomic DNA, denaturing the unmethylated genomic DNA fragments, incubating the denatured and unmethylated genomic DNA fragments under conditions conducive to the formation of snap back DNA by genomic DNA fragments including the DNA palindrome, and identifying regions of genomic DNA containing the DNA palindrome and the methylated DNA to form a profile. The method can further include comparing the profile of genomic DNA including a DNA palindrome and methylated DNA of the cancer cell population to a population of normal cells or to a profile established for another tumor type.

The present disclosure further provides methods for detecting a population of cancer cells. The methods can include isolating genomic DNA from a cell population, identifying a plurality of genomic DNA regions including methylated DNA and snap back DNA including a palindrome, and using the identity of the plurality of genomic DNA regions including the methylated DNA and palindrome to detect the population of cancer cells. The methods can further include fragmenting the isolated genomic DNA, denaturing the unmethylated genomic DNA fragments, incubating the denatured and unmethylated genomic DNA fragments under conditions conducive to formation of snap back DNA including the DNA palindrome, digesting denatured, single strand DNA, and identifying a plurality of regions of the genomic DNA containing the DNA palindrome and the methylated DNA to form a profile. The method can also include comparing the profile of the cancer cell population to a population of normal cells, wherein the cancer cell population includes genomic DNA including the DNA palindrome and the methylated DNA.

Methods for determining a region of genomic DNA that include an unmethylated CpG island are disclosed. The methods can include digesting genomic DNA with a methylation sensitive restriction enzyme, amplifying the DNA fragments using a labeled linker sequence, and hybridizing the amplified DNA fragments to a genomic DNA library and identifying the genomic DNA region including the palindrome.

The present disclosure also provides methods for identifying a region of genomic DNA including a DNA palindrome. The methods can include isolating genomic DNA including the DNA palindrome or the methylated DNA from a population of cells; denaturing the isolated, unmethylated DNA; incubating denatured isolated DNA under conditions conducive to inducing formation of a snap back DNA rather than inter-molecular hybridization, the snap back DNA including the DNA palindrome; digesting the denatured, unmethylated DNA; isolating the methylated DNA and the snap back DNA; denaturing the methylated DNA and the snap back DNA; incubating the methylated DNA and the snap back DNA under conditions conducive to inducing formation of the snap back DNA; digesting the denatured methylated DNA; and identifying one or more regions of the genomic DNA including the snap back DNA thereby identifying one or more regions of the genomic DNA including the DNA palindrome. The methods can include denaturation of methylated DNA by methods including alkaline denaturation or heating and an agent capable of lowering the melting temperature of methylated DNA, wherein such agent can include formamide.

Methods for isolating genomic DNA including a methylated DNA are disclosed. The methods can include the steps of incubating isolated genomic DNA under conditions conducive to hybridization of the methylated DNA and to denaturation of an unmethylated DNA; digesting the unmethylated DNA; and isolating the genomic DNA including methylated DNA. The methods can further include identifying regions of the genomic DNA including methylated DNA as well as additional steps including incubating the isolated genomic DNA under conditions conducive to inducing formation of a snap back DNA rather than inter-molecular hybridization, wherein the unmethylated DNA includes a DNA palindrome capable of forming snap back DNA; isolating the methylated DNA and the unmethylated DNA including the DNA palindrome; and denaturing the unmethylated DNA including the DNA palindrome. In certain embodiments, the denatured, unmethylated DNA can be digested with a single strand nuclease.

The present disclosure also includes methods for identifying CpG densities and degrees of CpG methylation in one or more regions of genomic DNA. The methods can include the steps of isolating genomic DNA; denaturing the isolated, unmethylated DNA; digesting the unmethylated DNA; isolating the genomic DNA including methylated DNA; and enriching for regions of genomic DNA having a specific CpG density and degree of CpG methylation. In certain embodiments, the methods can further include denaturing the genomic methylated DNA under a temperature, a concentration of formamide, and a concentration of NaCl tuned for hybridization of one or more regions of genomic DNA having a specific CpG density and degree of CpG methylation; digesting the denatured genomic methylated DNA; and, identifying the undigested regions of genomic DNA including methylated DNA

### BRIEF DESCRIPTION OF THE DRAWINGS

FIGS. 1A through C provide results of a series of experiments with a cell line including a large palindrome of the DHFR transgene (D79IR-8 Sce2 cells, WO 03/029438, incorporated herein by reference) demonstrating that the genome-wide assessment of palindrome formation assay efficiently generate intra-molecular base pairings in large palindromic sequences (‘snap-back’ DNA or SB DNA) and that these can be used to isolate large palindromic fragments from total genomic DNA. FIG. 1A depicts the NaCl-dependent formation of ‘snap-back’ (SB) DNA. Genomic DNA obtained from the CHO DHFR-cells containing inverted duplication of the DHFR transgene was heat denatured and rapidly cooled on ice. KpnI or XbaI digestion of DNA and Southern blotting demonstrated efficient intra-strand hybridization of the duplicated region. A 5 kb fragment of KpnI digest and an 11 kb fragment of XbaI digest, respectively, each of which is the size expected for the snap back DNA, were seen on the Southern blot in a NaCl-dependent manner. Solid lines and dotted lines represent single stranded DNA that was complimentary to each other. Probe used for hybridization is indicated on the figure. FIG. 1B depicts the same genomic DNA from D79IR-8 Sce2 cells as in FIG. 1A which was digested with SalI. The SalI-digested DNA was denatured, renatured, and subjected to S1 digestion. The double-stranded DNA was then digested with MspI or TaqI and the digested DNA was amplified by ligation-mediated PCR using linker specific primers. The DNA products were analyzed by Southern blot with a probe for a fragment that contains an inverted repeat (Probe 1), or a probe to an adjacent region that did not contain an inverted repeat (Probe 2). Signals were detected exclusively with the probe to the fragment with the inverted repeat (Probe 1), indicating that DNA obtained by this method is highly enriched for genomic sequences with palindromes. FIG. 1C examines whether the measurement of somatic palindromes could minimize the effect of non-palindromic counterpart(s). SalI-digested genomic DNA from D79IR-8 Sce2 and parental cells were mixed in a variety of ratios such that the total amount of DNA was 4 μg Two micrograms of DNA were subjected to snap back and amplification by LM-PCR for PCR-Southern analysis (upper panel), and the remaining 2 μg of the mixed DNA was digested with KpnI and analyzed by genomic Southern analysis (lower panel). Both Southern analyses were hybridized with a probe specific for inverted repeat (Probe 1 from FIG. 1B). Unlike the signals on the genomic Southern blot, specific signals from the palindrome were seen even after 1/40 dilution, indicating that this approach can detect somatic palindrome formation in a subpopulation of cells.

FIG. 2 is a pictorial summary of the “Procedure of Genome-wide analysis of Palindrome Formation” (GAPF). Tumor samples were subjected to the process to produce snap back DNA, treated with single strand specific nuclease S1, digested with either MspI, TaqI or MseI, ligated with a specific linker having the appropriate complementary sequence (MspI, TaqI or MseI), and amplified by PCR with Cy5-labeled linker specific primer. Standard DNA was prepared from normal human fibroblast (HFF) DNA by the same method except for the snap back process, and labeled with Cy3. Labeled DNAs were co-hybridized onto a human spotted cDNA microarray.

FIG. 3 depicts various comparisons of GAPF features between normal human fibroblasts, normal breast epithelial cells, epithelial cancer cell lines, and the pediatric cancers medulloblastoma and rhabdomyosarcoma. FIG. 3A compares the features of three normal human fibroblast preparations. No significant difference in GAPF features between normal human fibroblasts were observed. Features of SB-DNA of three independent primary cultures of fibroblasts (HDF1 (skin biopsy), HFF2 (foreskin sample) and HFF3 (skin biopsy)) were compared with non-SB-DNA of HFF2 as the common standard, genomic DNA of HFF2 without denaturation and renaturation (non-SB-DNA). Experiments were carried out in triplicate for each set of hybridization using three different preparations of templates. For each gene in each comparison, the q-value, which is a measure of significance in terms of false discovery rate (FDR), was calculated. In these analyses, thresholding genes with q-value<0.1 calls no genes significantly different between any two normal fibroblasts samples. The values pi(0), which represents the percentage of true negatives, and the minimum q-value (qₘᵢₙ) indicate that two sets of SB-DNA (HDF1 and HDF3) are almost identical, while that of HFF2 was very closely related to those of HDF1 and HDF3. FIG. 3B examines cancer specific somatic palindrome formations. GAPF features from HFF2 (normal human foreskin fibroblast, three independent hybridizations on microarrays, N=3), AG32 (normal breast epithelial cell line, N=3), HDF3 (normal human fibroblast, independent from FIG. 3A, N=5), Colo320DM (colon cancer cell line, N=3), MCF7 (breast cancer cell line, N=3), RD (rhabdomyosarcoma cell line, N=3) and five independent medulloblastoma tissues were compared to a common baseline profile consisting of two triplicate data sets of SB-DNA from HDF1 and HDF3 (FIG. 3A). The data from individual genes was grouped into 521 cytogenetic bands, and bands with q<0.05 and log(fold change)>0 were called ‘significantly increased’ relative to the common baseline. Numbers between each cell line and common baseline represent the number of significantly increased cytogenetic bands relative to the common baseline in the cell line. FIG. 3C examines the overlaps in areas of palindrome formation. Significant overlaps of somatic palindrome containing bands were found among age-related epithelial cancers (Colo320DM and MCF7, p=4.4427×10⁻⁶) or pediatric cancers (medulloblastomas and RD, p=0.017). FIG. 3D examines the distribution of overlaps of palindrome containing cytogenetic bands between age-related epithelial cancers and pediatric cancers. Neither Colo320DM nor MCF7 showed significant overlap of palindrome-containing cytogenetic bands with those of medulloblastoma or RD.

FIGS. 4A through 4C depict the clustering of somatic palindromes at specific regions of the genome in Colo320DM and MCF7. Genes from each loci and the surrounding region were plotted on the physical map and fold change of the GAPF and CGH (comparative genomic hybridization) features relative to HDF and are shown. Arrows indicate significant increases (q<0.05) either in Colo (black) or MCF7 (grey). FIG. 4A depicts the profiles of a 32 mega-base regions of the long arm of chromosome 8. The somatic palindromes commonly clustered in two regions at 8q24.1. Palindromes commonly cluster at the MYC gene and 5 MB centromeric to MYC. Note that palindrome formation was associated with the copy number increase of MYC, but not the genes at 5 MB centromeric in Colo320DM. FIG. 4B depicts the profiles of the 18 MB region at 1q21 and a detailed profile of the 4 MB clustered region. The data demonstrate a common cluster of somatic palindromes at a 600 kb region at 1q21. FIG. 4C depicts the palindrome profile of the region corresponding to the common fragile site Fra7I at 7q35.

FIGS. 5A and 5B depict a comparison of the snap back DNA profiles for a human foreskin fibroblast cell population and the human colon cancer cell line Colo320DN. FIG. 5A. The human colon cancer cell line Colo320DM contains an inverted duplication of the c-myc gene. Left panel; Southern blotting analysis of genomic DNA from either Colo320DM or human foreskin fibroblast (HFF). DNA rearrangement is seen in the Colo320DM. Denaturation and rapid renaturation (snap back, SB) of HFF DNA shows loss of the EcoRI fragment. Right panel; Genomic DNA from Colo320DM was either: (a) digested with EcoRI and then subjected to snap-back (EcoRI→SB); or, (b) subjected to snap-back and then digested with EcoRI (SB→EcoRI). Digesting with EcoRI prior to snap-back disrupts the inverted repeat following denaturation and results in fragments that will remain single stranded following snap-back and will be sensitive to S1 nuclease. In contrast, when snap-back is performed prior to EcoRI digestion, the intact inverted repeat will efficiently form double stranded DNA through intra-strand pairing, producing S1 nuclease resistant fragments following EcoRI digestion. Southern hybridization was done using a human c-myc cDNA probe. FIG. 5B. The ECM1 gene was amplified as an inverted repeat and was subjected to snap back. Southern analysis of SB-DNA from Colo320DM shows a half-size EcoRI fragment relative to that of non-SB-DNA, indicating a palindromic amplification of ECM1. Right panel; A human myogenin probe was cohybridized as a control. Left panel; no fragment was seen on the SB-DNA from Colo320DM DNA by hybridizing with the myogenin probe only.

FIG. 6 depicts the hierarchical clustering of the GAPF profile of 5 medulloblastomas and three normal fibroblasts (HDF3). A high degree of similarity among five individual medulloblastomas was seen, which is clearly separable from normal fibroblasts.

FIG. 7 is an idiogram showing genome wide distribution of somatic palindromes. Palindrome-containing cytogenetic bands are shown on the right side of chromosome (Colo320DM, left column of circles, and MCF7, right column of circles) or on the left side (medulloblastoma, right column of circles, or RD, left column of circles). The cytogenetic bands with palindromes that are identified in both Colo and MCF7 cluster at 1q21, 8q24.1, 12q24, 16p12-13.1 and 19q13.

FIGS. 8A and 8B provide a schematic and data for using ligand-mediated methylation PCR to amplify DNA fragments enriched for unmethylated CpG islands. FIG. 8A provides a schematic for the process of ligand-mediated methylation PCR for amplification of unmethylated CpG islands. FIG. 8B provides a blot showing the amplification of small (<500 base pair) HpaII DNA fragments.

FIG. 9 provides a general schematic of the genome-wide analysis of palindrome formation (GAPF) assay, also alternatively depicted in FIG. 2. Genomic DNA was first digested with either KpnI or SbfI, and then these reactions were combined. Palindromic sequences can rapidly anneal intramolecularly to form ‘snap-back’ DNA under conditions that do not favor intermolecular annealing. This snap-back property was used to enrich for palindromic sequences in total genomic DNA by denaturing the DNA at 100° C., rapidly renaturing it in the presence of 100 mM NaCl, and then digesting the mixture with the single-strand specific nuclease S1. Snap-back DNA formed from palindromes was double-stranded and resistant to S1, whereas the remainder of genomic DNA is single-stranded and thus was sensitive to S1 digestion. Ligation-mediated PCR was performed, and then the DNA was labeled and hybridized to a microarray for analysis.

FIG. 10 illustrates exemplary results from a genome-wide analysis of palindrome formation (GAPF) assay that can identify DNA palindromes. FIGS. 10A and 10B illustrate a tiling array analysis of GAPF-positive regions in Colo320DM (Colo) cells compared to primary skin fibroblasts (HDF). Graphically displayed are signal (log₂(signal ratio); top graph and dark gray), and p-values (−10 log₁₀; bottom graph and light gray). The solid dark gray bars below the top graph depict log₂(signal ratio)>1.5 where Colo>HDF, and the solid light grey bars below the bottom graph depict (−10 log₁₀) p-values>30 (=p<0.001). FIG. 10A depicts a GAPF-positive signal of the known palindrome at the CTSK locus. Signal was observed to within approximately 300 by of the known junction between one of the palindromic arms and the nonpalindromic center (junction depicted by double-headed arrow). FIG. 10B depicts a GAPF-positive signal at the known palindrome at ECM1.

FIG. 11 illustrates that nonpalindromic GAPF-positive loci were recalcitrant to a second round of GAPF but denature in the presence of 50% formamide. FIG. 11A shows a PCR-based enrichment assay after one round (GAPF×1) or two rounds (GAPF×2) of GAPF in Colo320DM (Colo). The assay was performed in duplicate. PCR products using unprocessed genomic DNA (gDNA) were included for comparison. As a negative control, the PCR product labeled as Tel amplified a region on chromosome 1 that does not contain a DNA palindrome, and primers to generate this fragment were added for multiplex PCR in each of the loci evaluated. The palindrome at the CTSK locus was enriched after one round of GAPF, but did not survive the second round. Seven non-palindromic loci (CDH2, DNAJA4, HAND2, KCNIP4, NRG1, OPCML and PHOX2B) survived the second round of GAPF. FIG. 11B illustrates that formamide addition during denaturation optimized the assay for DNA palindromes. PCR-based enrichment assay is shown. GAPF was performed in Colo cells with either no modification (GAPF) or with 50% formamide (50% Form) in the denaturation step. Both the palindrome at the CTSK locus and a naturally occurring inverted repeat (IR6-107.3) present in the human reference genome were enriched. Signal from two non-palindromic loci (HAND2 and OPCML) were largely abolished with the addition of 50% formamide. The assay was performed in duplicate. The PCR product marked Tel served as a negative control.

FIG. 12 generally provides results depicting that formamide can enhance GAPF specificity for DNA palindromes, as provided by a tiling array analysis of GAPF-positive regions in Colo320DM (Colo) cells compared to primary skin fibroblasts (HDF). Each panel graphically displays p-values (−10 log₁₀; top graph and light gray) and signal (log₂(signal ratio); bottom graph and dark grey). The solid bars below the top and bottom graph depict (−10 log₁₀) p-values>30 (=p<0.001) and log₂(signal ratio)>1.5 where Colo>HDF, respectively. The top pair of light gray and dark gray graphs depict the results from the original GAPF method, and the bottom pair of lightdepicts the results from GAPF with the addition of 50% formamide. FIG. 12A depicts tiling array data shown for nonpalindromic loci. The addition of 50% formamide abolished these signals. FIG. 12B illustrates that the palindromes at CTSK and ECM1 were enhanced by the addition of 50% formamide. FIG. 12C depicts a putative palindromic region on chromosome 13 encompassing the genomic region between PDX1-PRHOXNB.

FIG. 13 shows that nonpalindromic GAPF-positive loci identify regions of CpG DNA methylation. A bisulfite DNA sequence analysis is shown for individual clones from either Colo320DM (Colo) or primary fibroblasts (HDF). Black circles represent CpG methylation, and white circles depict unmodified CpG dinucleotides.

FIG. 14 depicts an exemplary schematic of an analysis used to assay the genome for methylation. In this assay the regions of methylated DNA do not denature while unmethylated DNA denature; upon rehybridization under rapid renaturation conditions the unmethylated DNA failed to rehybridize and is digested with a nuclease specific for single strand nucleotide sequences. The double stranded methylated DNA regions are not digested. In this embodiment, linkers are added to the double stranded methylated DNA regions, and the regions are amplified by PCR, biotin labeled and used to hybridize to a DNA arranged on microarray for detection.

FIG. 15 illustrates that differential denaturation can identify CpG methylation at previously described loci in HCT116 cells, as shown by a promoter tiling array analysis of positive regions in HCT116 compared to DKO cells. Each panel graphically displays signal (log₂(signal ratio; top graph and dark grey)) and p-values (−10 log₁₀; bottom graph and light grey). The solid bars below the top dark gray graph depict log₂(signal ratio)>1.2 where HCT116>DKO. The solid bars below the bottom light gray graph depict (−10 log₁₀) p-values>30 (=p<0.001).

FIG. 16 illustrates that differential denaturation can be used to identify common loci among primary medulloblastoma samples. FIG. 16A depicts methylation-positive gene detection. FIG. 16B depicts methylation-negative genes from four primary medulloblastoma samples (R123, R147, R160 and R162), as identified on the Affymetrix™ Promoter Array. Cerebellum from one normal individual was used as a control. Total number of methylation-positive or methylation-negative loci for each sample is shown, and common regions between the four samples are depicted on the Venn diagram. FIG. 16C depicts a bisulfite sequence analysis of the PTCH1-1C methylation-positive promoter region for one of the medulloblastoma samples (R 160) and the normal cerebellum control.

### DETAILED DESCRIPTION

The present disclosure describes methods for conducting analyses of DNA methylation and DNA palindrome formation. For example, the disclosed methods can be used for genome-wide analyses of DNA methylation and DNA palindrome formation at different regions of genomic DNA. The parent application, U.S. patent application Ser. No. 11/142,091, to the present disclosure includes the description of a novel method described as Genome-wide Analysis of Palindrome Formations (GAPF). These methods were believed to identify genomic DNA including a DNA palindrome. The present disclosure is based in-part on the unexpected discovery that the genomic DNA resulting from practicing the GAPF method as disclosed in the parent application can result in a population of genomic DNA including a palindrome but also includes a population of genomic DNA having regions of methylated DNA. The result is based on the unexpected property of methylated DNA to not fully denature under what has been believed to be standard conditions capable of denaturing all genomic DNA, e.g., heating to 100° C. in 100 mM salt. In particular, although the presence of 5-methylcytosine is known to increase the melting temperature (TO of DNA, it has been generally accepted that all DNA, even methylated DNA, fully denatures under such conditions. In accordance with this unexpected discovery, the present disclosure describes methods for the enriching for genomic DNA including methylated DNA and a DNA palindrome.

Alternatively, some of the disclosed methods can be used to enrich for genomic DNA including a DNA palindrome. In other embodiments, methods are disclosed that can be used to enrich for genomic DNA including methylated DNA. Still further, methods are disclosed that comprise differential denaturation that can enrich for varying levels of DNA methylation that is generally referred to as Methylation Analysis by Differential Denaturation (MADD). In addition, the disclosed methods can be adapted to amplify DNA enriched for unmethylated CpG islands. The methods further provide procedures to identify chromosomal regions susceptible to subsequent gene amplification associated with cancer and other conditions. Such methods can serve as sensitive techniques to detect early stages of tumorigenesis since in many cases chromosome aberration are early manifestations of malignant transformation.

Certain methods described herein offer advantages over other existing methods for identifying regions of DNA methylation. For example, the method designated as Methylated DNA Immunoprecipitation (MeDIP) can be problematic because the antibodies used in the method only recognize single-stranded DNA and thus may miss regions of the genome that are heavily methylated and resistant to efficient DNA denaturation. In certain embodiments, the disclosed methods can enrich for methylated DNA because such DNA remains double-stranded while the unmethylated (or less methylated) DNA sequence denature, and the denatured DNA is sensitive to digestion with a single strand nuclease such as 51 nuclease. The denaturation conditions used for MeDIP are similar, if not less stringent, than those used in the disclosed methods. Thus, the disclosed methods can advantageously identify a subset of CpG-methylated loci that is likely never detected using standard MeDIP protocols.

Another potential advantage for the detection of DNA methylation using the disclosed methods is that the methods are qualitative, rather than quantitative in nature like some of the existing genome-wide DNA methylation assays. This gives the presently disclosed methods the potential to sensitively detect aberrant DNA methylation associated with disease-specific DNA methylation changes from very few cells in a background of normal cells or tissue. It is also possible to ‘tune’ the disclosed methods to enrich for different amounts of DNA methylation across the genome. At the most stringent practice, the disclosed methods can efficiently identify heavily methylated loci. In addition, by adjusting salt concentration, denaturation temperature, and formamide concentration, the methods can identify a gradient of CpG methylation densities.

In addition to bettering understanding of the process of carcinogenesis, the loci identified by the disclosed methods can serve as useful biomarkers of disease. By generating disease-specific DNA methylation signatures, the development of clinical assays based on the disclosed methods can aid in: early detection of disease, disease diagnosis, measurement of response to treatment, and evaluation of minimal residual disease monitoring for disease recurrence. For each of these applications, an initial loci or set of loci can be identified by the disclosed methods or any other genome-wide assay. The low cost and high sensitivity of the disclosed methods, however, suggests one or several of the methods could be a method for clinical applications to determine the methylation status of informative loci in patient samples.

Generally, the nomenclature used herein and many of the laboratory procedures in regard to cell culture, molecular genetics and nucleic acid chemistry and hybridization, which are described below, are those well known and commonly employed in the art. (See generally Sambrook et al., Molecular Cloning: A Laboratory Manual, 3d Ed., Cold Spring Harbor Laboratory Press, New York (2001), which is incorporated by reference herein). Standard techniques are used for recombinant nucleic acid methods, preparation of biological samples, preparation of cDNA fragments, PCR, and the like. Generally enzymatic reactions and any purification and separation steps using a commercially prepared product are performed according to the manufacturers' specifications. Although specific enzymes and other recombinant nucleic acid methods and products are described and used, other enzymes and recombinant nucleic acid methods and products are well known in the art and are available for use in the described methods.

The methods described herein generally use genomic DNA from any cell population, tissue sample, and the like. Cell populations or tissue samples that can be used in the methods include any normal tissue, such as skin, blood, bladder, lung, prostate, brain, ovary, and the like, a tumor, such as a melanoma, leukemia, bladder tumor, lung tumor, prostate tumor, brain tumor, ovarian tumor, and the like, or any other tissue or organ at a particular point in development.

### Methods for Enrichment of DNA Palindromes and Methylated DNA

Loss of chromosome integrity in human cancers generates numerous gains and losses of chromosome segments. Large DNA palindromes caused by Breakage-Fusion-Bridge (BFB) cycles might facilitate gene amplification in human cancers, however, the prevalence of initial palindrome formation is largely unknown. In the present disclosure, novel methods are used to demonstrate that somatic palindrome formation and methylated DNA are widespread and non-random in human cancers. Individual tumor types appear to have a characteristic distribution of palindromes in their genome and only a subset of these palindromic or methylated loci are associated with gene amplification. The present disclosure identifies widespread palindrome formation and methylated DNA in human cancer that can provide a platform for subsequent gene amplification and indicates that tumor specific mechanisms determine the locations of palindrome formation and/or DNA methylation. A method for rapidly identifying the genomic DNA locations of palindrome formation and/or methylated DNA in various populations of cells is provided herein, as well as applications of the methods for characterizing tumor types, palindrome and/or methylated regions susceptible to gene application and their association with cancer diagnosis and early cancer detection, assessment of residual disease, and monitoring for disease recurrence.

Provided herein is a novel microarray based approach to assay palindromes and/or DNA methylation in genomic DNA. By using this approach it has been found that somatic palindrome formation is in fact a common form of chromosome instability and that these palindrome formations tend to cluster at specific loci in the genome, “hotspots for palindrome formation.” In addition, the methods have been found to efficiently detect regions of DNA methylation using assay conditions previously thought to destroy the double-strandedness of such regions. Surprisingly, use of the methods disclosed herein has revealed that individual tumor types appear to have a characteristic distribution of palindromes and/or methylated DNA in their genome, indicating that tumor specific mechanisms determine the locations of palindrome formation and/or DNA methylation. Somatic palindromes are not always associated with significant gene amplification, whereas loci with high-level amplifications are usually accompanied by somatic palindromes. These data indicate that the somatic formation of palindromes broadly alters the cancer genome and provides a platform for subsequent gene amplification. DNA methylation on the other hand is known to be a characteristic of tumorigenesis. The present methods provide a simple efficient means to detect and localize DNA methylation.

In certain embodiments of the present disclosure, the methods can be used for identifying genomic DNA including methylated DNA and/or a DNA palindrome. For example, the methods can include steps of isolating genomic DNA, fragmenting the genomic DNA, and denaturing the genomic DNA. Due to the discovered higher melting temperature of methylated DNA, certain denaturation conditions can be used to selectively denature unmethylated DNA. For example, unmethylated DNA fragments can include DNA fragments having a DNA palindrome and other DNA fragments that do not include a DNA palindrome or methylation (e.g., nonpalindromic DNA). Genomic DNA can be isolated using any of a variety of methods known generally in the art. In certain embodiments of the present disclosure, genomic DNA can be isolated from a population of cells, such as normal or cancerous cells. Fragmentation methods are similarly well known in the art and can include chemical, physical, or enzymatic methods. Methods for denaturing the genomic DNA can depend on the desired purpose of a given method. Generally, denaturation can be achieved through specific temperature conditions, such as heating to about 100° C., and with or without addition of a salt, such as NaCl. Salt concentrations can range from approximately 1-500 mM, and more typically from approximately 1-100 mM. Denaturation conditions can also include addition of other agents that can affect the melting temperature of DNA, such as a DNA helix destabilizing agent, e.g., formamide. Previous studies, for example, have shown that for every 1% of formamide, the DNA melting temperature can be reduced by approximately 0.6-0.72° C. (Hutton, Nucleic Acids Research, 4:3537-3555 (1977); McConaughy et al., Biochemistry 8:3289-3295 (1969)).

Following a denaturation step, the genomic DNA can be incubated under conditions that disfavor intermolecular hybridization and instead favor formation of snap back DNA by DNA fragments having a DNA palindrome. For example, the genomic DNA can be denatured by boiling and then rapidly cooled, or renatured, in the presence of 100 mM NaCl by cooling in an ice water bath. Subsequently, the methylated DNA, which does not denature under such conditions, and DNA having a DNA palindrome will be double-stranded and thus resistant to digestion by a single strand nuclease, such as S1 nuclease. Addition of a single strand nuclease can then digest the remaining single strand DNA, leaving intact the genomic DNA including methylated DNA and a DNA palindrome.

Known methods in the art, such as micro-array techniques, can be used to further identify regions of the genomic DNA that include a methylated DNA and/or a DNA palindrome. For example, human genomic DNA arrays can be used to quantitatively and qualitatively analyze the genomic DNA. These arrays can include, for example, DNA hybridization assays including high-density oligonucleotide arrays, such as Affymetrix™ GeneChip® Human Tiling Arrays, that can have probes tiled at an average resolution of 35 basepairs across the genome. Such arrays can sample a large genome DNA library to qualitatively analyze the regions of genomic DNA that include methylated DNA (e.g., contain CpG islands) and/or regions that include a DNA palindrome.

In some embodiments, the disclosed methods can also include amplification of the genomic DNA prior to genome-wide analyses. For example, samples containing genomic DNA fragments including methylated DNA and/or a DNA palindrome can be prepared for amplification by digesting the double stranded DNA fragments including a DNA palindrome with a nucleotide sequence specific restriction enzyme, such as MspI, TaqI, or MseI. A sequence specific linker nucleotide can then be added to the end of double stranded DNA. The DNA fragments including the added linker can be amplified using a labeled linker sequence specific primer that corresponds to the sequence specific linker. In certain embodiments, the amplified DNA fragments can be further mixed and co-hybridized with a sample of high molecular weight DNA from a normal cell population that has been digested with single strand nuclease, such as S1 nuclease, and the restriction enzyme, has added linkers labeled with a second single label, and has been amplified. In each of these embodiments, the amplified DNA fragments can then be hybridized to a genomic DNA array as described above to identify regions of the genomic DNA having methylated DNA and/or a DNA palindrome.

### Methods for Enrichment of Genomic DNA Having a DNA Palindrome

The present disclosure includes methods for enrichment of genomic DNA including a DNA palindrome. In certain embodiments, the disclosed methods can further be used to identify regions of genomic DNA including a DNA palindrome. In an exemplary embodiment, genomic DNA can be isolated and fragmented using methods described herein and known to one of ordinary skill in the art. Generally, the fragmented genomic DNA includes methylated DNA and unmethylated DNA that includes non-palindromic DNA and DNA having a DNA palindrome. Enrichment for palindromes can be achieved by denaturing the fragmented DNA and subsequently incubating the denatured, fragmented DNA under conditions that disfavor intermolecular hybridization and instead favor formation of snap back DNA by DNA having a DNA palindrome. The denaturation conditions can, also, be adjusted to lower the melting temperature of methylated DNA. The addition of a DNA helix destabilizer, for example, formamide, to a solution including the DNA during denaturation can lower the melting temperature by approximately 0.6-0.72° C. for approximately every about 1% of formamide that is added. Thus, methylated DNA can be denatured under certain conditions that depend on the density of DNA methylation. For example, lightly methylated DNA can denature under lower concentrations of the DNA helix destabilizer, whereas more heavily methylated DNA can require a higher concentration of the DNA helix destabilizer. Accordingly, in a specific embodiment, a range of concentrations of the DNA helix destabilizer formamide, such about 0-50% or more can be used. Furthermore, temperature and salt concentration can be tuned to target certain densities of DNA methylation. In one exemplary embodiment, the denaturation step can include boiling in water at about 100° C. in the presence of about 50% formamide to lower the DNA melting temperature by approximately 30° C. Under these conditions, methylated DNA can be denatured, remain single-stranded when rapidly cooled, and then subsequently digested by a single-stranded nuclease, such as S1 nuclease. Similarly, denatured non-palindromic DNA can be digested by a single-stranded nuclease. DNA having a DNA palindrome, in contrast, will still form snap-back DNA in the presence of formamide, and when rapidly cooled, will remain S1-resistant. Given that non-palindromic DNA and methylated DNA have been digested, the isolated genomic DNA will be enriched for genomic DNA including one or more DNA palindromes. This genomic DNA can then be assayed using methods described herein to determine regions of the genome that contain a DNA palindrome.

In an alternative embodiment, denaturation of methylated DNA can be achieved by other methods besides heat and formamide, such as alkaline denaturation, with for example, NaOH or KOH (Ageno et al., Biophysic. J. 9:1281-1311, 1969; Levinson et al., Am. J. Med. Genet. 51:527-534, 1994). After neutralization and rehybridization under snap back conditions, methylated DNA would remain single-stranded and thus S1-sensitive, while the intramolecular annealing of palindromic DNA would still occur and produce an S1-resistant species. Upon enrichment of DNA having a DNA palindrome, the regions of genomic DNA including such palindromes can be identified using the methods described herein.

### Methods for Enrichment of Methylated Genomic DNA

The present disclosure also includes methods for the enrichment of methylated DNA. The differential denaturation methods that can be used to analyze CpG DNA methylation as described herein are generally referred to as Methylation Analysis by Differential Denaturation (MADD). These methods can include certain steps as described above. In an exemplary embodiment, methylated DNA can be enriched by performing two successive cycles of denaturation/renaturation/single-strand nuclease digestion. The first cycle can enrich for both palindromic and methylated DNA, while the second cycle enriches for methylated DNA. Methylated DNA that was resistant to denaturation during the first cycle will remain double-stranded (and thus, e.g., S1-resistant) during the second cycle of denaturation. In contrast, palindromic DNA will not survive the second denaturation/renaturation cycle, since the initial non-palindromic DNA loop holding the arms of the palindrome together is digested by the single-strand endonuclease in the first round. During the second denaturation step, intramolecular annealing of the palindrome is not possible because of the loss of the physical connection provided to the arms of the palindrome by the non-palindromic loop region. Accordingly, the palindromic DNA is subsequently digested by a single strand nuclease, such as S1 nuclease, thereby leaving only the methylated DNA. In certain embodiments, an additional purification step can be performed by removing the DNA helix destabilizer, e.g., formamide, and performing a denaturation/renaturation/S1 digestion cycle to clean-up the reaction, thereby also enriching for the methylated DNA.

An alternative embodiment that enriches for methylated DNA can take advantage of the relative stability of S1 nuclease to both temperature and formamide. S1 retains its nuclease activity up to approximately 65° C. and approximately 50% formamide. In certain embodiments, the single-strand specific endonuclease, such as S1 nuclease, retains activity at higher temperatures and formamide concentrations. Under these conditions, most of the genomic DNA will become single-stranded, or at the least, the DNA double-helix will ‘breathe’ to form regions of single-strandedness. Palindromic DNA will also have these characteristics, and thus will be degraded in the presence of a single strand specific nuclease. Methylated DNA, because of its increased melting temperature in comparison to the palindromic DNA, will remain double-stranded and thus resistant to digestion by the endonuclease.

Embodiments that enrich for methylated DNA can further be used to identify genomic regions including methylated DNA. Given that unmethylated DNA is digested by the above methods, the genomic DNA isolated will be enriched for fragments that are methylated. This genomic DNA can then be assayed to determine which regions of the genome contain the methylated DNA using the methods described herein.

In certain embodiments of the methods disclosed herein, genomic DNA from a cell population or tissue sample is digested with a methylation sensitive restriction enzyme. Methylation sensitive restriction enzymes useful in the present disclosure include, for example, HpaII, and the like. Prior to digestion the genomic DNA can be fragmented by known physical, chemical or enzymatic means to form high molecular weight DNA. The high molecular weight DNA can then be further digested with the methylation sensitive restriction enzyme.

Methods for Enriching Methylated DNA with Varied Degrees of Methylation

In certain embodiments of the present disclosure, methods can be used to enrich for methylated DNA having varied degrees of methylation or in combination with varied degrees of CpG densities. For example, the disclosed methods can be modified to affect the thermal denaturation kinetics of DNA in order to ‘tune’ the assay to enrich for different degrees of DNA methylation and CpG content. These modifications can include performing the denaturation at a range of formamide concentrations, a range of salt (e.g., NaCl) concentrations, and at a range of different temperatures. In some embodiments, varying the concentration of formamide over a small window (0.1% to 1% final concentration) at 100° C. can enhance the melting temperature difference between different degrees of DNA methylation at regions of relatively high CpG content, e.g., CpG islands.

In addition, the range of CpG content and degree of CpG methylation differentially detected can be extended by varying the NaCl and/or formamide concentrations, while heating the DNA over a range of temperatures below 100° C. For example, a range between 90-100° C. in very low salt conditions, for example, 0 to about 10 mM, can be used to distinguish methylation differences in regions of lower CpG content or regions that have a lower percentage of CpG methylation when compared to denaturation conditions that distinguish unmethylated from heavily methylated CpG islands, for example, at about 100° C. and about 100 mM NaCl.

In other embodiments, the methods disclosed herein can be extended to identify a broad range of differences in the degree of CpG methylation at regions with a broad range of CpG content, e.g., regions that are not CpG islands. For example, the amount of salt and formamide concentrations can be varied to achieve a differential DNA melting temperature for a range of CpG content and methylation. In certain embodiments, DNA can be incubated at about 65° C. (or at a range of temperatures) and at different concentrations of formamide in which identical DNA sequences will have different melting temperatures based on CpG methylation. Theoretically, conditions can be set to distinguish any desired degree of difference in overall DNA methylation. In addition to distinguishing differences in the overall degree of methylation at a broad range of CpG content, the methods can be further adjusted to determine the methylation state of CpG residues in a given DNA context (e.g., in the context of a transcription factor or insulator factor binding site) on a genome-wide basis. Such methods can be achieved, for example, by adding a single strand nuclease, such as S1, at the time of heating the DNA in the presence of a concentration of salt and formamide designed to distinguish the melting temperature of an unmethylated and a methylated sequence.

In certain embodiments, the methods disclosed herein can be used to interrogate the genome for varying degrees of methylation at regions of varying CpG content relative to a reference sample (e.g., cancer to non-cancer). To achieve detection of differential methylation at a broad range of CpG content, a series of DNA samples can be assayed over a range of salt, formamide, and temperatures. For example, under the relatively stringent conditions (e.g., about 100° C. with about 100 mM NaCl) regions with a “high” CpG content and relatively heavy methylation can be distinguished from regions with low methylation. At lower stringencies (e.g., temperatures lower than about 100° C. with varying amounts of salt and formamide), regions with lower CpG content can be interrogated for methylation status. Under these lower stringency conditions, regions with “high” CpG content cannot be distinguished based on methylation because neither will denature.

In other embodiments, the stringency of the conditions can be modified in either a step-function or as a continuous gradient to identify regions with different CpG densities and degrees of CpG methylation. DNA enriched under different stringency conditions can be differentially labeled (e.g., with different fluorochromes or quantum dots) and hybridized to the same array of nucleotides, e.g., DNA fragments. By these methods, methylation status can be identified by reading which label (corresponding to a given condition) hybridizes to a given locus. Alternatively, DNA prepared under different conditions can be labeled or segregated and queried using other methods (e.g., sequencing). In these manners, genome-wide assessment of varying degrees of DNA methylation at regions with a broad range of CpG content can be obtained.

In yet other embodiments, the disclosed methods can also identify areas of the genome with different degrees of methylation and CpG density. Bisulfite sequencing has been performed on the regions of genomic DNA giving the strongest positive signals confirming that indeed the identified areas of the genome contained methylated DNA. There are many other statistically significant positive loci (>200) that have been identified using the methods of the present disclosure and tiling arrays comprising genomic DNA that map to regions of the genome with varying degrees of CpG density. It is quite possible that the degree of DNA methylation will also be varied among these loci.

### Methods for Analyzing a Population of Cancer Cells

The methods described in the present disclosure can be used to study populations of cells and, for example, to compare cancer cells to normal cells. In one embodiment of the present disclosure, the methods described herein can be used to classify a population of cancer cells. For example, certain methylated DNA or DNA palindromes can be associated with a certain cancer cell and not present in normal cells. Once one or more regions of genomic DNA are identified to have methylated DNA and a snap back DNA including a DNA palindrome, these marker regions can be used to classify the population of cancer cells.

In another embodiment of the present disclosure, the methods described herein can be used to detect a population of cancer cells, for example, by comparing a profile of methylated DNA and DNA palindromes identified in cancer cells versus a profile characteristic of normal cells. In certain embodiments, a profile can include analyzing one or more regions of genomic DNA that indicate a positive or negative result for the presence of a DNA palindrome. Other embodiments can include profiling one or more regions of genomic DNA including methylated DNA. In yet another embodiment, profiles can be associated with cancer cells or normal cells based on the analysis of one or more regions of genomic DNA including methylated DNA and a DNA palindrome. As described herein, the methods for detecting a population of cancer cells can include steps described elsewhere in the present disclosure, such as isolating genomic DNA from a cell population, identifying one or more genomic DNA regions including methylated DNA and snap back DNA including a palindrome, and using the identity of the one or more genomic DNA regions including methylated DNA and a palindrome to detect the population of cancer cells.

### Methods for Enrichment of Unmethylated CpG Islands

Ligation-mediated PCR (LM-PCR) can also be used to amplify DNA enriched for unmethylated CpG islands. The method can be used, for example, to study differential methylation between cancer and normal cells, and tissue specific methylation during differentiation. The method generally can use genomic DNA from any cell population, tissue sample, and the like. The cell population or tissue samples that can be used in the method include any normal tissue, such as skin, blood, bladder, lung, prostate, brain, ovary, and the like, a tumor, such as a melanoma, leukemia, bladder tumor, lung tumor, prostate tumor, brain tumor, ovarian tumor, and the like, or any other tissue or organ at a particular point in development. Genomic DNA from a cell population or tissue sample is digested with a methylation sensitive restriction enzyme. Methylation sensitive restriction enzymes useful in the present disclosure include, for example, HpaII, and the like. Prior to digestion the genomic DNA can be fragmented by known physical, chemical or enzymatic means to form high molecular weight DNA. The high molecular weight DNA can then be further digested with the methylation sensitive restriction enzyme.

### EXAMPLES

### Example 1

The following example describes a process for genome-wide assessment of palindrome formation.

### Methods

### Cell Lines and Cancer Tissues

D79IR-8 and D79IR-8-Sce 2 cells were previously described (Tanaka et al., Proc. Natl. Acad. Sci. USA 99:8772-8777 (2002)). Colo320DM and RD were obtained from American Type Culture Collection. MCF7 and AG 1113215 were from the University of Washington. Skin biopsy derived fibroblasts HDF1 and HDF3 were obtained from the University of Washington and human foreskin fibroblasts HFF2 from the Fred Hutchinson Cancer Research Center (FHCRC) as anonymous cell lines. DNA samples stripped of identifying information from five primary medulloblastomas were provided by the Fred Hutchinson Cancer Research Center. All samples were obtained after Fred Hutchinson Cancer Research Center Institutional Review Board review and approval for use of anonymous human DNA samples and human cell lines.

### Linkers and Oligonucleotides

Oligonucleotides were synthesized by QIAGEN™ Genomics. For ligation mediated PCR, two oligonucleotides were annealed in the presence of 100 mM NaCl; for MspI digested DNA, JW102 g -5′-GCGGTGACCCGGGAGATCTGAATTG-3′ (SEQ ID NO:1) and JW103 pc2-5′-[Phosp]CGCAATTCAGATCTCCCG-3′ (SEQ ID NO:2), for TaqI digested DNA, JW102-5′-GCGGTGACCCGGGAGATCTGAATTC-3′ (SEQ ID NO:3) and JW103p2 5′-[Phosp]CGGAATTCAGATCTCCCG-3′ (SEQ ID NO:4), and for MseI digested DNA, JW102 g- and JW103 pcTA -5′-[Phosp]TACAATTCAGATCTCCCG-3′ (SEQ ID NO:5). To label DNA for microarray, the following linker specific primers were end-labeled either with Cy3 or Cy5 and used for PCR; for MspI linker ligated DNA, JW102gMSP -5′-GCGGTGACCCGGGAGATCTGAATTGCGG-3′ (SEQ ID NO:6), for TaqI linker ligated DNA, JW102Taq -5′-GCGGTGACCCGGGAGATCTGAATTCCGA-3′ (SEQ ID NO:7), for MseI linker ligated DNA, JW102gMse -5′-GCGGTGACCCGGGAGATCTGAATTGT AA-3′ (SEQ ID NO:8).

To make a probe for Southern analysis, human genomic DNA was amplified by PCR and a fragment was cloned (TOPO TA Cloning® Kit (Invitrogen™)). Oligonucleotides used for PCR were; for ECM1, ECM15154, 5′-ACACCTTTCACACCTCGCTTCTC-3′ (SEQ ID NO:9) and ECM15851 5′-GGCAGATAAAGAAGAGACAGTGGTTG-3′ (SEQ ID NO:10).

### Microarray Analysis

To make a snap-back DNA, 2 μg of high molecular weight genomic DNA in 50 μl with 100 mM NaCl was boiled for 7 minutes and transferred on ice to cool it down quickly. 6 μl of S1 nuclease buffer, 4 μl of 3 M NaCl and 100 Units of S1 nuclease (Invitrogen™) was added to the DNA and incubated at 37° C. for about one hour. S1 nuclease was inactivated by 10 mM EDTA and phenol/chloroform extraction. DNA was precipitated by ethanol and dissolved in water and digested with 40 U of MspI, TaqI or MseI for 16 hours. DNA was precipitated, dissolved into 21 μl of water and ligated to a MspI, TaqI or MseI specific linker by adding 5 μl of 20 mM linker, 3 μl of T4 DNA ligase buffer and 400 U of T4 DNA ligase at 16° C. for about 16 hours. DNA was precipitated and dissolved into 200 μl TE, followed by being applied onto a centrifugal filter unit (MICROCON YM-50; Millipore™) to remove any excess of linker. DNA was recovered in 20 μl water. Thus for each cell line or tumor tissue, templates with three different linkers were prepared. For PCR, 2 μl of DNA, 0.5 μl of Taq DNA polymerase (FASTSTART Taq DNA polymerase; Roche™), 2.5 μl of 2 mM dNTP, 5 μl of 10×PCR buffer, 2 μM of a Cy3 or Cy5 labeled linker-specific primer were mixed with water to a total of 50 μl reaction. PCR was performed at 96° C. for 6 minutes followed by 30 cycles of 96° C. for 30 sec, 55° C. 30 sec and 72° C. 30 sec on a 9600 Thermal Cycler (Perkin-Elmer™). PCR reactions for the same template from different linker specific primer were mixed and purified (PCR purification Kit; QIAGEN). Human Cot-1 DNA (100 μg), poly polydA/dT (20 μg), and yeast tRNA (100 μg) were added for hybridization to a 18 k human cDNA array. For primary medulloblastoma, each tumor sample was processed as a singleton and the GAPF profiles from the five independent samples were compared to the human foreskin cell sample (HDF) GAPF profile. To prepare template DNA for array-CGH analysis, genomic DNA was digested with MspI, TaqI or MseI, and ligated with a linker specific for each restriction enzyme. Three independent preparation of template DNA were amplified either by Cy3 or Cy5 labeled linker-specific primer. Triplicated co-hybridization of either Cy3-labeled cancer (Colo320DM or MCF7) DNA with Cy5-labeled normal (HFF2) DNA or Cy5-labeled cancer DNA with Cy3-labeled normal DNA was performed. Oligonucleotides were synthesized by QIAGEN Genomics.

### Southern Blotting

Southern blotting was performed as described previously. Briefly, 2 μg of high molecular weight human genomic DNA was digested with restriction enzyme, run on 0.8% agarose gel and blotted to nylon membrane. Snap-back DNA was prepared as follows; 2 μg of genomic DNA in 50 μl water with 100 mM NaCl was boiled for 7 minutes and immediately transferred on ice to be cooled down. DNA was precipitated by ethanol, and digested with restriction enzyme. 2.5 kb Molecular Ruler (BIO-RAD), 1 kb DNA ladder and 100 by DNA ladder (New England Biolabs™) were used as size markers. To make a probe for Southern analysis, human genomic DNA was amplified by PCR and a fragment was cloned by TOPO TA Cloning Kit® (Invitrogen™) as described above.

### Statistical Analysis

Array data was normalized in the GeneSpring™ Analysis Package, version 6.2 (Silicon Genetics™, Redwood City, Calif.) using Lowess normalization (an intensity-dependent algorithm). The data was then transformed into logarithmic space, base 2. Data was annotated by cytogenetic band or by UniGene cluster using NCBI databases current as of February, 2004. Welch's t-test was performed for each cytogenetic band or UniGene cluster comparing replicate data sets. Storey's q-value was used to control for multiple testing error and each p-value was transformed to a q-value, which is an estimate of the false discovery rate.

### Results

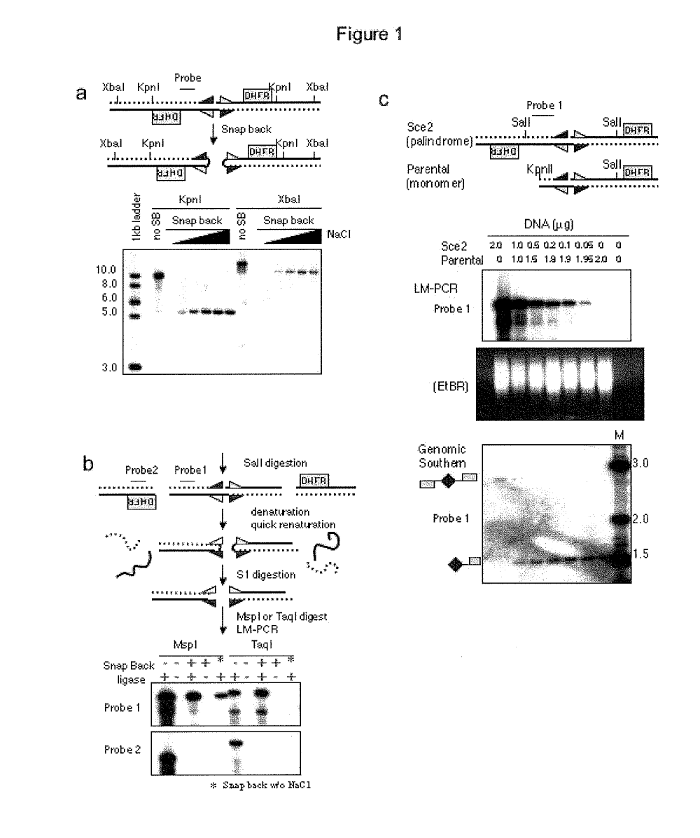

A method to obtain a genome-wide assessment of palindrome formation is disclosed herein based on the efficient generation of intra-molecular base pairing in large palindromic sequences. (Ish-Horowicz et al., J. Mol. Biol. 142:231-245 (1980); Ford and Fried, Cell 45:425-430 (2986). Palindromic sequences can rapidly anneal intramolecularly to form “snap-back” (SB) DNA under conditions that do not favor inter-molecular annealing. Snap-back DNA formation can be demonstrated from an endogenous palindrome after heat denaturation and rapid cooling of genomic DNA from cells that contain a few copies of a large palindrome of the DHFR transgene (D79-8 Sce2 cells) (FIG. 1A). The decreased size of the restriction length fragment—the 11 kb KpnI fragment becomes 5.5 kb and the 24 kb XbaI fragment becomes 12 kb, respectively—indicates that renaturation occurs through intramolecular base-pairing.

To determine whether the efficient formation of snap-back DNA could be used to isolate large palindromic sequences from total genomic DNA, genomic DNA from D79-8 Sce2 cells was digested with SalI, followed by denaturation, rapid-renaturation, and digestion with the single strand specific nuclease S1. The snap-back DNA formed by palindromes should be relatively resistant to S1 nuclease, whereas the remainder of the genomic DNA will not efficiently re-anneal and should be S1 sensitive (FIG. 1B). S1 resistant double-stranded DNA was amplified by ligation-mediated (LM) PCR using linker-specific primers after digestion with MspI or TaqI and detected by Southern blotting with either a probe within the inverted repeat (probe 1) or a probe in an adjacent non-palindromic fragment (probe 2). A signal was detected exclusively with the probe to the palindromic fragment, indicating that the genomic DNA obtained by this method was highly enriched for palindromic sequences. This also demonstrated that the enrichment depended on the structure of the DNA, not the copy number of the gene, because the copy number was the same for the fragment with the inverted repeat and the adjacent non-palindromic fragment.

A dilution experiment was performed to demonstrate that this technique can identify genomic palindromes that exist in a sub-population of cells, such as might occur in a tumor with a heterologous population of genetically altered cells, such as provided by an intratumoral heterogeneity. Genomic DNA from D79IR-8 Sce2 cells was serially diluted with DNA from the parental cells that contained a single non-palindromic copy of the transgene. The DNA mixes were analyzed by standard genomic Southern analysis (FIG. 1C, lower panel) or subjected to snap-back, amplification by LM-PCR, and then Southern analysis (FIG. 1C, upper panel). Using a probe specific to the inverted repeat (probe 1 from FIG. 1B), specific signal from the palindrome was seen even after a 1/40 dilution, demonstrating that this approach can detect a somatic palindrome in a sub-population of cells.

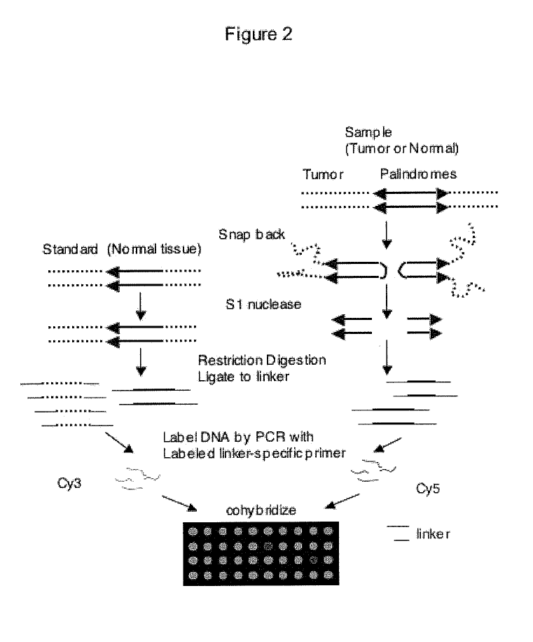

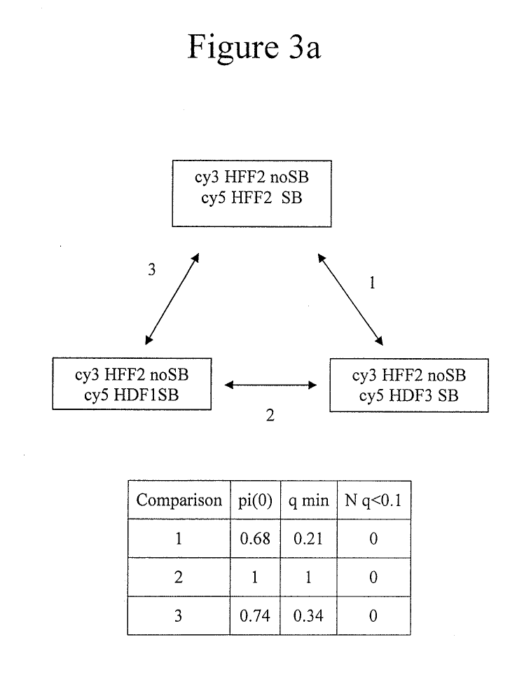

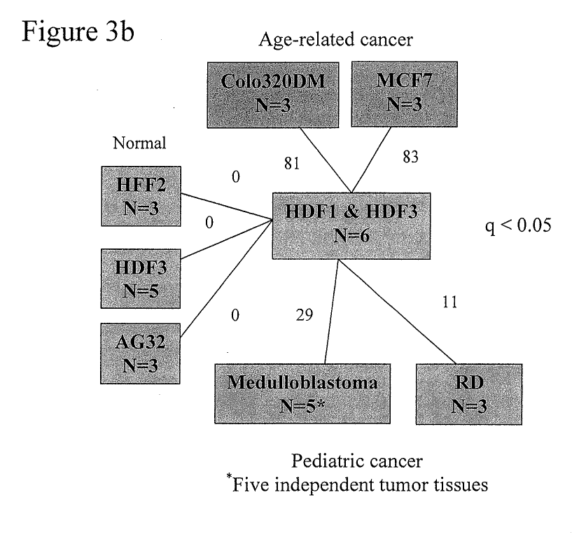

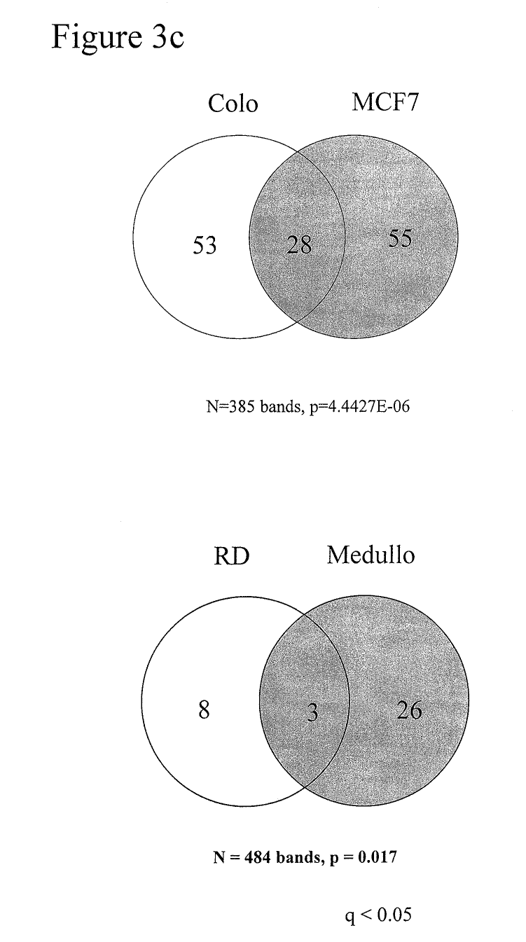

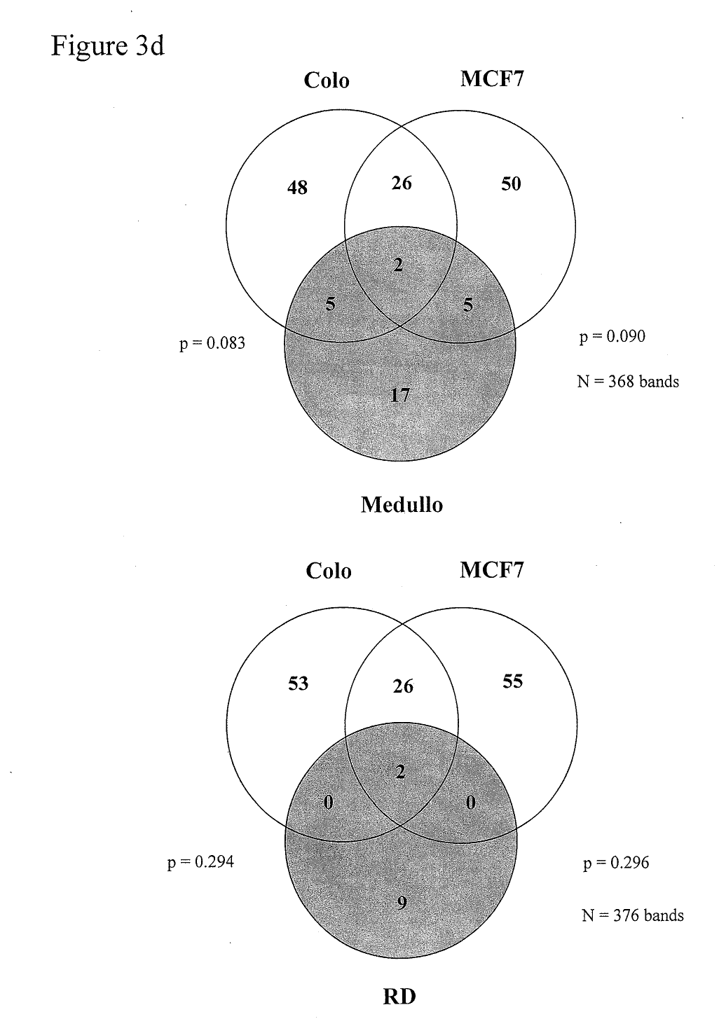

With this technique, genome-wide analysis of palindrome formation (GAPF) can be assessed using DNA array hybridization. Initially, genomic DNA was used from primary cultures of human fibroblasts derived from three different individuals (HDF1 (skin biopsy), HFF2 (foreskin sample) and HDF3 (skin biopsy)). It was assumed that somatic DNA palindrome formation was related to genetic instability and that normal fibroblasts would not have many differences between them. Genomic DNA from each of the fibroblasts was subjected to denaturation and rapid-renaturation (snap-back, or SB DNA); digested with S1 nuclease and restriction enzymes (MspI, TaqI or MseI); ligated to a linker specific for each enzyme; and amplified by PCR amplification with Cy-5 labeled linker specific primers (FIG. 2). For the common standard competitor DNA, genomic DNA was used from similarly processed HFF2 fibroblasts but without denaturation (non-SB DNA) and amplified using Cy-3 labeled linker specific primers. Cy-3 labeled non-SB HFF2 DNA was competitively hybridized against Cy-5 labeled SB DNA from HFF2, HDF1, or HDF3 on spotted arrays containing 18,000 (18k) human cDNAs, generating comparable GAPF profiles of fibroblasts from each individual. For each fibroblast DNA, three independent preparations of SB DNA were processed for hybridization. The Storey's q-value, a measure of significance in terms of false discovery rate (FDR), was calculated for each gene in each comparison between fibroblasts to control for multiple testing errors. At a threshold of q<0.1, no features showed a significant difference between any two of the normal fibroblast samples (FIG. 3A).

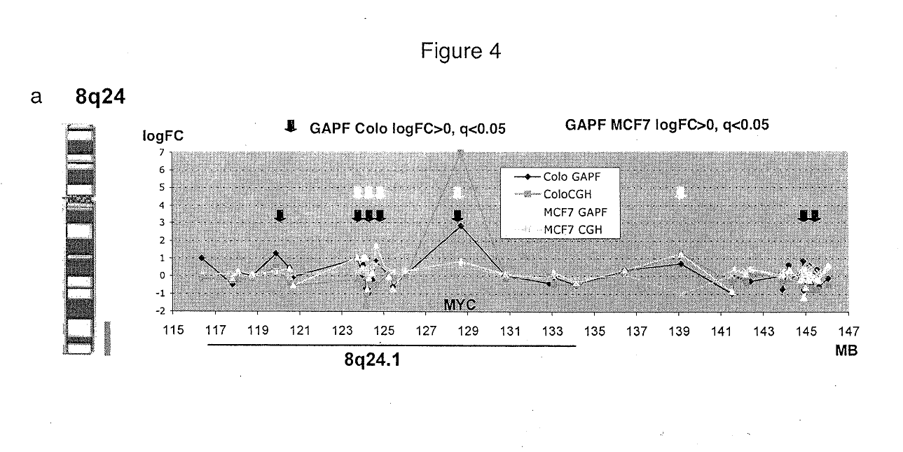

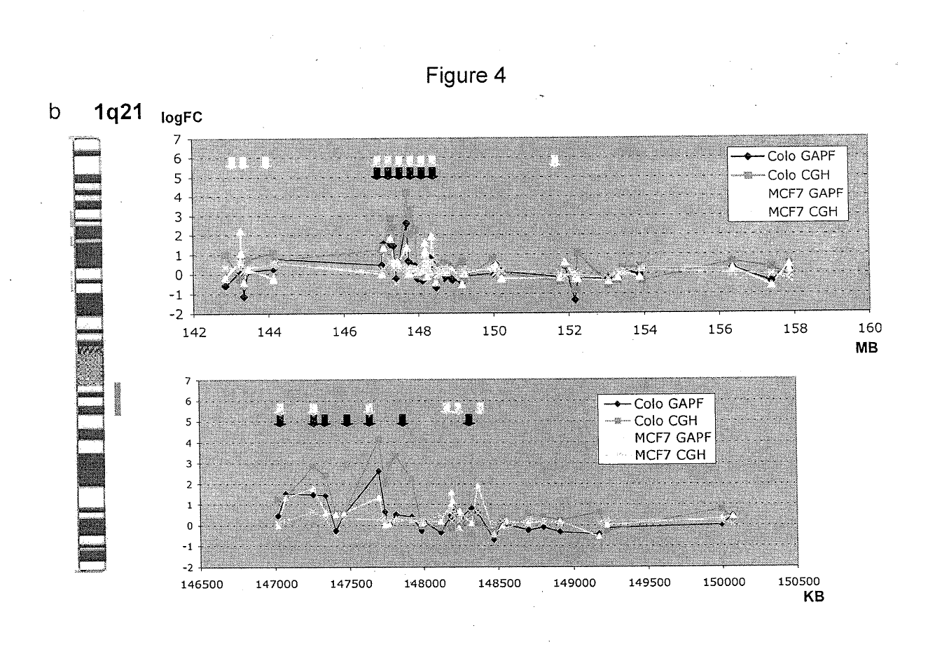

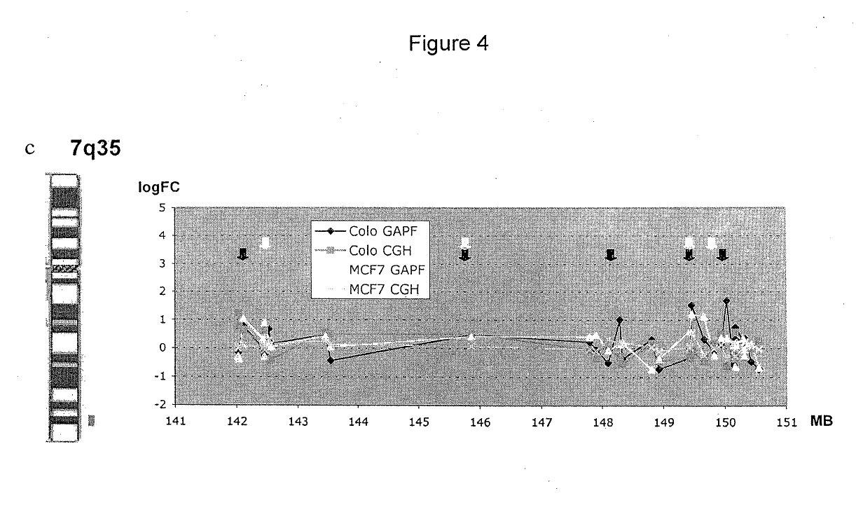

To determine whether GAPF can detect palindromes formed in cancer cells, the Colo320DM human colon cancer cell line (Colo) that has a large inverted repeat of the cMyc gene was used initially. SB DNA from Colo was labeled with Cy-5 and co-hybridized with the Cy-3 labeled non-SB DNA of HFF2 fibroblast. Experiments were performed in triplicate and the GAPF profile was compared to a ‘common baseline’ GAPF profile consisting of two triplicate data sets of SB DNA from the HDF1 and HDF3 fibroblasts (FIG. 3B). For this analysis, the data from individual genes was grouped into 521 cytogenetic bands that ranged in size from 1 to 132 genes with an average of 18 genes per cytogenetic band. Locating each gene on a physical map of cytogenetic bands helped to identify regions susceptible to palindrome formation. Based on a criteria of a q-value<0.05 and a log-fold change>0, there were no differences between the common baseline and the HFF2 GAPF, whereas 81 cytogenetic bands were increased in the Colo GAPF (FIG. 3B), indicating increased numbers of palindromes in the Colo DNA when compared to normal fibroblast DNA. As predicted, the cytogenetic band that includes cMyc, 8q24.1, showed a significant increase in Colo (q=0.024). This band covers 18 genes in a 13 Mb region and the increased features show a bimodal distribution: cMyc is GAPF-positive and there was also a cluster of three genes (ZHX2, MGC21654, and annexin A13) in an approximately 900 kb region located 5 MB centromeric to cMyc that are also GAPF-positive (FIGS. 4A and 5A), which is consistent with a previous report that cMyc is amplified as a large inverted repeat in this cell line. A similar clustering of GAPF increased genes was also identified at 1q21 (FIG. 4B). This cytogenetic band was significantly increased in Colo (q=5.53×10⁻⁵), with three individual genes (Histone 2 (HIST2H2BE), vacuolar protein sorting 45A (VPS45A) and extracellular matrix protein 1 (EMC1), CKIP1 and FLJ23221) clustering within 600 kb (FIGS. 4B and 5B). Two additional genes (CK2 interacting protein 1 and FLJ23221) with a significant increase are also assigned to this region, indicating that this subregion of a cytogenetic band was a hotspot for palindrome formation.

For comparison, a GAPF profile was obtained for a breast cancer cell line, MCF7, a normal breast epithelial cell line (AG 11132), and a rhabdomyosarcoma cell line, RD. No cytogenic bands were GAPF-positive in the comparison of AG 11132 with the normal HDF fibroblast baseline, whereas eighty-three cytogenetic bands and 73 bins were significantly increased in MCF7 relative to the HDFs (FIG. 3B), including both 8q24.1 (q=0.035) and 1q21 (q=0.0056). At 8q24.1, the increased genes were the same four as are increased in the Colo cells (FIG. 5A). At 1q21, the increased genes include three that were also increased in Colo (Histone 2 (HIST2H2BE), Vacuolar protein sorting 45A (VPS45A) and Extracellular matrix protein 1 (ECM1)) (FIG. 4B). Overall, there was a significant overlap of the palindrome containing cytogenetic bands in Colo and MCF7 (28 bands, p=3.4427×10⁻⁶ and 20 bins, p=4×10⁻⁶) (FIG. 3C), indicating that these epithelial tumor cell lines from age-related cancers have common hotspots of palindrome formation. Similar to the analyses based on cytogenic bands or bins, there is also a significant overlap of GAPF-positive genes between Colo (150 genes) and MCF7 (388 genes) (40 genes in common, p<1×10⁻⁹⁹).

The GAPF profile of the RD cell line, derived from an embryonal rhabdomyosarcoma, identified 11 palindrome-containing cytogenetic bands. These 11 bands do not show significant overlap with those of Colo (p=0.29) or MCF7 (p=0.29), indicating that distinct GAPF patterns were associated with different types of tumor cells. It is interesting that the 2q35 band was identified as containing a palindrome in RD cells and the PAX3 gene in this region was enriched but did not meet the preset statistical criteria to be independently called elevated. Alveolar rhabdomyosarcomas are characterized by a t(2; 13)(q35; q14) translocation that fuses the PAX3 gene with the FKHR gene on chromosome 13, whereas embryonal rhabdomosarcomas do not carry this translocation; however, the association of this region with a somatic palindrome formation in an embryonal rhabdomosarcoma indicates that PAX3 resides in a GAPF hotspot in this cell type and suggested that the alternative resolutions of a double-stranded break at this hotspot might determine the subtype of rhabdomyosarcoma generated.

Interestingly, the formation of palindromes at the GAPF hotspots was not always associated with an increase in gene copy number, as measured by comparative genomic hybridization (array-CGH). For example, at both 8q24.1 and 1q21, palindrome formation was associated with a significant increase (more than two-fold) in copy number in Colo but not in MCF7. In Colo, the cMyc associated palindrome at 8q24.1 was amplified, whereas the cluster of palindrome embedded genes in the adjacent region 5 MB centromeric to cMyc was not amplified. This discrepancy between the GAPF profile and array-based CGH indicates that the two approaches are measuring different features in the cancer cells: GAPF measures a structural feature (palindrome) and CGH measures the average copy number. In fact the majority of the genes that are significantly increased by GAPF in Colo were not identified as increased by CGH; however, GAPF genes were significantly more likely to be amplified than other loci, indicating that a subset of GAPF loci were selected for amplification. These data suggest that BFB cycles drive tumor progression by forming somatic palindromes at the specific loci, some of which are selected for gene amplification. For example, two of the three Colo loci (8q24.1 and 1q21) that include genes with more than a three-fold increase in copy number by CGH were associated with palindrome formations by GAPF. Also, the DUSP22 gene, another gene that shows more than three-fold amplification at 6p25 by array-CGH was associated with palindrome formation at the gene level, although 6p25 itself was not identified as a palindrome-containing cytogenetic band based on our predetermined statistical criteria. In contrast, at 7q35, where a common fragile site (FRA7I) is implicated as a chromosome break site in the palindromic amplification of the PIP oncogene in a breast cancer cell line, a gene (Contactin associated protein-like 2) has a palindrome formation in both Colo and MCF7 with a low-level increase in copy number in Colo, whereas two other genes (Zinc finger protein 289 and potassium voltage-gated channel, subfamily H) demonstrated palindromes in Colo with a low-level decrease in copy number. These data indicated that unstable hotspots in the cancer genome resulted in clustered areas of palindrome formation that serve as a platform for gene amplification.

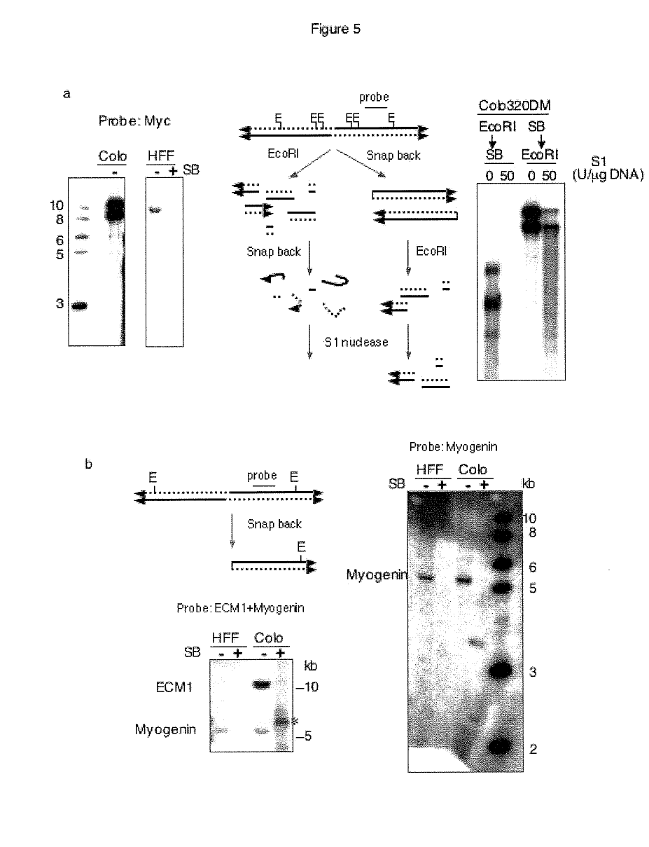

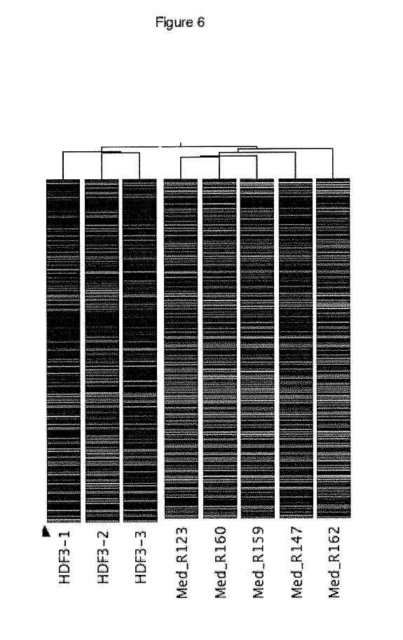

Colo, MCF7, and RD are cell lines derived from primary tumors and it is possible that the widespread palindrome formation revealed by GAPF might be secondary to multiple passages in culture. To examine somatic palindrome formation in primary tumors, GAPF analysis was performed on DNA isolated from five independent primary medulloblastomas, the most common central nervous system malignancy of childhood. Each tumor sample was processed as a singleton and the GAPF profiles from the five independent samples compared to the HDF GAPF profile. Somatic palindrome formation was detected at 29 cytogenetic bands in the primary human medulloblastomas (q<0.05) (FIG. 3B) and hierarchical clustering showed a high degree of similarity among individual medulloblastomas, which have a GAPF pattern that was clearly similar to each other and distinct from Colo and MCF7 (FIG. 6 and FIG. 3D). These palindrome-containing loci include 6q (6q12, 6q14), 4q (4q24, 4q25) and 7q (7q21.1, 7q22.1 and 7q31), which were commonly amplified in medulloblastoma tissues. Other GAPF-positive loci, such as 1p34.2, 5p15.2, 5p15.3 and 13q34, have been identified as highly amplified loci in a subset of medulloblastomas, suggesting a link between gene amplification and palindrome formation. The fact that five independent primary tumors have common loci of somatic palindrome formation indicates a shared mechanism of palindrome formation and indicated that tumor specific mechanisms determine their genomic location. It was interesting to note that the palindromic regions contained genes that likely contribute to tumor progression: Skp2 at 5p13 encodes a subunit of ubiquitin ligase complex that regulates entry into S phase by inducing the degradation of the cyclin dependent kinase inhibitors p21 and p27; Fzd1 at 7q21.1 encodes a receptor for the Wnt signaling pathway that is often dysregulated in medulloblastomas; and, Tert, telomere reverse transcriptase at 5p15.3 is often amplified in medulloblastomas.

In contrast to the similarity of the Colo and MCF7 GAPF profiles, there was no significant overlap of cytogenetic bands between medulloblastomas and Colo320DM (p=0.08) or between medulloblastomas and MCF7 (p=0.09); however, significant overlap was evident between medulloblastomas and RD (p=0.01) (FIG. 3C), despite the much smaller number of palindrome containing cytogenetic bands in RD. These results indicated a different distribution of somatic palindromes in pediatric tumors (medulloblastomas and rhabdomyosarcomas) and age-related cancers (colon and breast), suggesting that the mechanisms responsible for palindrome formation at specific loci might reflect fundamental properties of tumor cell biology.

### Discussion

These results identify widespread somatic palindromes that occur in characteristic patterns in specific cancer types. Unlike conventional array-CGH (comparative genomic hybridization) analysis that measures the average gene dosage in cell populations, GAPF provides a qualitative measurement of a structural chromosomal aberration (palindromes) that has previously been examined only by cytogenetic studies. Detailed mapping of the palindromes on the physical genome reveals that palindrome formations tend to cluster at specific regions, some of which undergo gene amplification. In addition, the pattern of genome wide palindrome formation appears to be different among different types of cancers, indicating that the palindrome formation reflects specific differences in the biology of each cancer type.

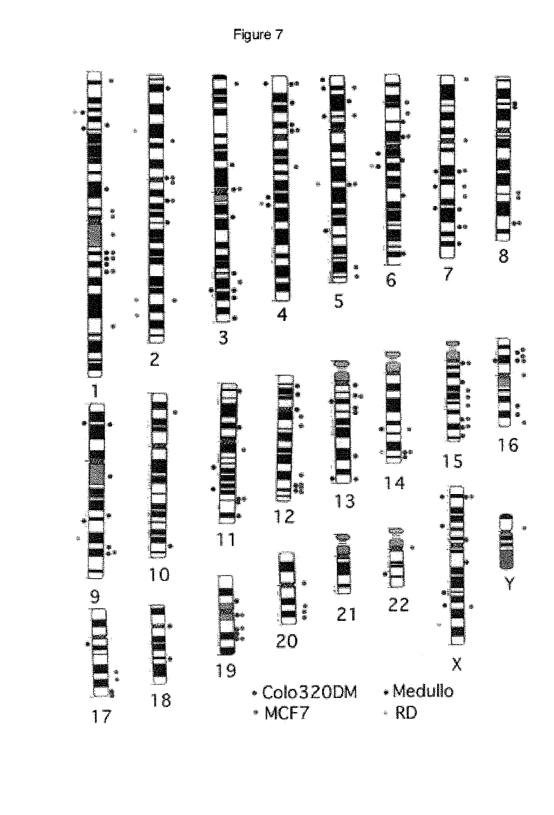

The clustering of somatic palindromes could be due to clustering of chromosome breakage sites in the genome, since chromosome breakage is required for palindrome formation. Cytogenetic studies have shown that clastogenic drug-induced fragile sites are involved in inverted duplications and gene amplifications in rodent cells (Coquelle et al., Cell 89:215-225 (1997)), and aphidicolin-induced fragile sites are involved in oncogene amplification in human cancer cells (Ciullo et al. Hum. Mol. Genet. 11:2887-2894 (2002); Hellman et al., Cancer Cell 1:89-97 (2002)). In fact, the GAPF-positive cytogenetic bands detected in both the Colo320DM human colon cancer cell line and the MCF7 breast cancer cell line were co-localized at 1q21, 8q24.1, 12q24, 16p12-13.1 and 19q13, which all harbor common fragile sites (FIG. 7). Although the majority of the common fragile sites remain to be characterized at the molecular level, the fact that palindromes cluster at these loci suggests a role for common fragile sites in palindrome formation. Stability of common fragile sites is controlled, in part, by the replication checkpoint kinase ATR (Casper et al., Cell 111:779-789 (2002)). In yeast, impaired function of the ATR homologue Mce1 leads to stalled replication forks and chromosome breaks in specific regions of the genome (Cha and Kleckner, Science 297:602-606 (2002) that can result in gross chromosome rearrangement (Myung et al., Cell 104:397-408 (2001)). Compromised checkpoint function might generate similar chromosome breaks and somatic palindromes in specific regions of the genome in cancer cells. In addition to common fragile sites, topoisomerase cleavage sites might determine sites of initial DNA double strand breakage, which have been shown to initiate disease-associated chromosomal translocations (Domer et al., Proc. Natl. Acad. Sci. USA 90:7884-7888 (1993); Dong et al., Genes Chrom. Cancer 6:133-139 (1993); Hirai et al., Genes Chrom. Cancer 26:92-96 (1999); Lovett et al., Proc. Natl. Acad. Sci. USA 98:9802-9807 (2001); Obata et al., Genes Chrom. Cancer 26:6-15 (1999)). It is also interesting that a number of GAPF positive genes are associated with translocations in some tumor types, such as T-cell leukemia/lymphoma 1A (TCL1A) (Davey et al., Proc. Natl. Acad. Sci. USA 85:9287-9291 (1998); Erickson et al., Science 229:784-786 (1985); Hecht et al., Science 226:1445-1447 (1984)); Synovial sarcoma, X-breakpoint 4 (SSX4) (Skytting et al., J. Natl. Cancer Inst. 91:974-975 (1999), and Myeloid leukemia factor 1 (MLF1) (Yoneda-Kato et al., Oncogene 12:265-275 (1996)). Therefore, it is possible that chromosome breaks at these genes might be resolved either as a palindrome or as a translocation with significantly different consequences to the progression of the tumor.

In RD, 2q35 was identified as GAPF-positive and the PAX3 gene in this region was enriched by GAPF, although not meeting the present statistical criteria to be independently call elevated as a single gene. Alveolar rhabdomyosarcomas are characterized by a t(2; 13)(q35; q14) translocation that fuses the PAX3 with the FKHR gene on chromosome 13, whereas embryonal rhabdomyosarcomas do not carry this translocation (Anderson et al. Genes Chrom. Cancer 26:275-285 (1999)); however, the association of this region with a somatic palindrome formation in an embryonal rhabdomyosarcomas indicates that PAX3 resides in a GAPF hotspot in this cell type and suggests that the alternative resolutions of a double-stranded break at this hotspot might determine the subtype of rhabdomyosarcoma generated. For medulloblastoma, it is also interesting to note that the palindromic regions contain genes that might contribute to tumor progression: Skp2 at 5p13 encodes a subunit of ubiquitin ligase complex that regulates entry into S phase by inducing the degradation of the cyclin dependent kinase inhibitors p27 (Carron et al., Nat. Cell Biol. 1:193-199 (1999)); Fzd1 at 7q21.1 encodes a receptor for Wnt signaling pathway that is often dysregulated in medulloblastomas (Yokota et al., Int. J. Cancer 101:198-201 (2002)); and Tert, telomere reverse transcriptase at 5p15.3 is often amplified in medulloblastomas (Fan et al., Am. J. Pathol. 162:1763-1769 (2003)).

In addition to the requirement for a double-strand break, other cis-acting sequences might determine where palindromes can form. In the simple eukaryotes Tetrahymena (Butler et al., Mol. Cell. Biol. 15:7117-7126 (1995); Yao et al., Cell 63:763-772 (1990); Yasuda and Yao, Cell 67:505-516 (1991)), yeast, e.g., S. pombe (Albrecht et al., Mol. Biol. Cell 11:8730886 (2000)), and Leshmania (Grondin et al. Mol. Cell. Biol. 16:3587-3595 (1996)), palindrome formation is mediated by a pair of short inverted repeats that naturally exist in the genome. In S. cervisiae, exogenous short inverted repeats consisting of human Alu repeats inserted in the chromosome can induce chromosome breaks and palindrome formation in an Mre11 mutant background (Lobachev et al., Cell 108:183-193 (2002)). In CHO cells, it has been directly shown that short inverted repeats can mediate palindrome formation following an adjacent double-strand break, which leads to subsequent BFB cycles and gene amplification (Tanaka et al., Proc. Natl. Acad. Sci. USA 99:8772-8777 (2002)). Short inverted repeats are common in the human genome and are often involved in disease-related DNA rearrangements (Kurahashi and Emanuel, Hum. Mol. Genet. 10:2605-2617 (2002); Kurahashi et al., Am. J. Hum. Genet. 72:733-738 (2003)). Further studies might determine whether naturally occurring short inverted repeats facilitate the widespread palindrome formation that has been characterized in cancer cells.

Alveolar rhabdomyosarcomas are characterized by a t(2; 13)(q35; q14) translocation that fuses the PAX3 and FOXO1A genes on chromosome 13, whereas embryonal rhabdomyosarcomas do not carry this translocation; however, the association of this region with a somatic palindrome formation in an embryonal rhabdomyosarcoma RD implies that PAX3 also resides in a region susceptible to DSBs and suggests that the alternative resolutions of a DSB might determine the subtype of rhabdomyosarcoma generated.

Surprisingly, most of the loci with palindromes are not associated with an increase in gene copy number. In addition, the cancer cells from age-related epithelial cancers form palindromes at similar locations, whereas five different primary medulloblastomas have their own distinct pattern of palindrome distribution, which is similar to a pediatric rhabdomyosarcoma derived cancer cell line. It appears, therefore, that sets of cancer types share common profiles of palindrome formation. Subsequent gene amplification might occur at subsets of these loci given tumor-specific selective pressure for growth. For example, palindromes cluster at 1q21 and 8q24 in both Colo320DM and MCF7, however, copy number is increased only in Colo320DM. This indicates that palindrome formation might be an early and fundamental step in cancer formation, providing a platform for subsequent gene amplification at a restricted set of loci. In this model, different tumor types might have a common set of palindromes, but the selective advantage of a given locus would determine its subsequent amplification in the cancer. The identification of widespread palindrome formations specific to different types of cancers provides a new opportunity to develop sensitive assays for detection of residual disease, early detection, and tumor classification. Ultimately, preventing the underlying mechanisms that lead to widespread palindrome formation might prevent tumor initiation.

### Example 2

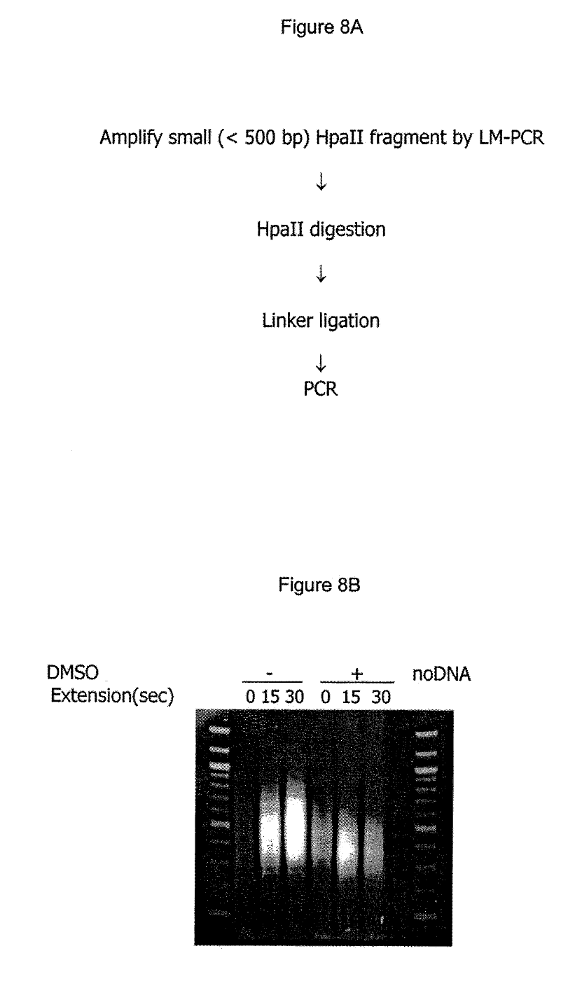

The following example demonstrates the use of ligation-mediated PCR to isolate a DNA fragment enriched in unmethylated CpG islands in a mammalian cell. A schematic of the process is provided as FIG. 8A.

Briefly, mouse genomic DNA was digested with a methylation sensitive restriction enzyme (for example, HpaII). The MspI linkers used above in Example 1 were used to ligate the HpaII fragments. The ligated DNA was amplified by PCR using the MspI primer from Example 1 (SEQ ID NO: 6). The method resulted in the specific amplification of HpaII digested genomic DNA of less than 500 base pairs (FIG. 8B). Random cloning and sequencing of the PCR products revealed that more than 50% of clones were at the CpG islands as defined using stringent criteria. (Takai and Jones, Proc. Natl. Acad. Sci. USA 99:3740-3745 (2002); incorporated herein by reference). In contrast, amplification of DNA digested with methylation-resistant isoschizomer MspI gave no clones near CpG islands.

TABLE 1
Results of random sequencing.

| | n | GC content | CpG Island |
|---|---|---|---|
| HpaII | 20 | 56.2% | 11 (55%) |
| | | (43-68%) | |
| MspI | 11 | 50.6% | 0 (0%) |
| | | (43-59%) | |

A systematic study of the methylation status of CpG islands throughout the genome becomes possible by combining this approach with human or mouse CpG island microarrays. For example, the labeled unmethylated DNA fragments can use to interrogate a microarray DNA library constructed from a particular organism or tissue from a particular organism. The result with this library can be compared to a DNA library constructed from a different tissue or the same tissue from a different developmental period. The differences between the methylation patter determined from each tissue sample can indicate changes in DNA methylation associate with, for example, tumorigenesis, or development.

### Example 3

The following example describes methods used to identify palindromes and methylated DNA.

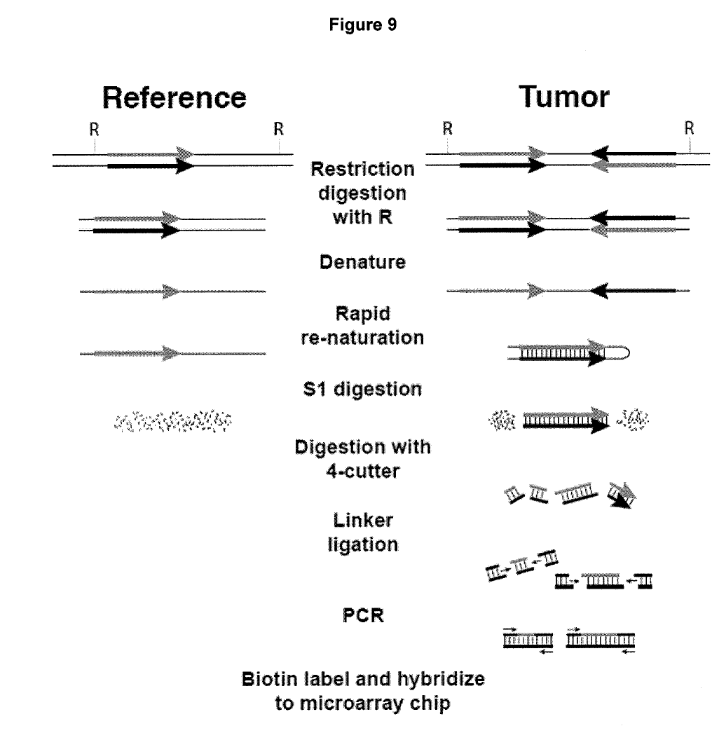

Above is described a method to obtain a genome-wide analysis of palindrome formation (GAPF) based on the efficient intrastrand base pairing in large palindromic sequences (Tanaka et al., Nat. Genet. 37:320-327 (2005)). Palindromic sequences can rapidly anneal intramolecularly to form ‘snap-back’ DNA under conditions that do not favor intermolecular annealing. This snap-back property was used to enrich for palindromic sequences in total genomic DNA by denaturing the DNA at 100° C. in the presence of 100 mM NaCl, rapidly renaturing it by snap cooling, and then digesting the mixture with a single-strand specific nuclease. Snap-back DNA formed from palindromes was double-stranded and resistant to the single-strand specific nuclease, whereas the remainder of genomic DNA was single-stranded and thus was sensitive to digestion (FIG. 9). Using this assay, de novo palindromes were shown to form in cancers (Tanaka et al., Mol. Cell. Biol. 27:1993-2002 (2007)), and that the GAPF-positive signal at the CTSK locus in Colo320DM cells represents a DNA palindrome that defines the border of an amplicon (Tanaka et al., Mol. Cell. Biol. 27:1993-2002 (2007)).

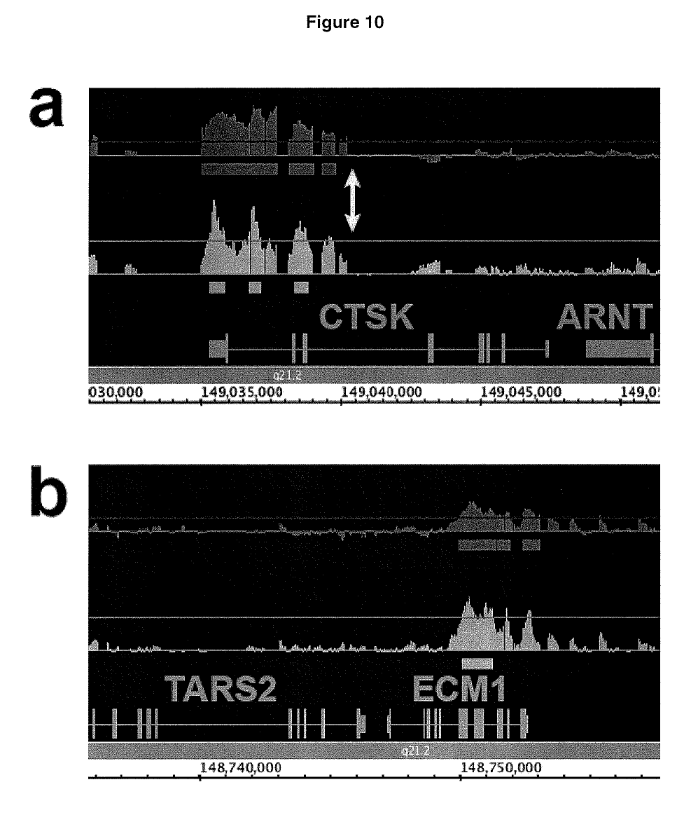

To facilitate the detailed mapping of DNA palindromes, the GAPF assay was performed as described in Example 1 on genomic DNA from Colo320DM cells (Colo) and control primary human diploid fibroblasts (HDF) and applied to high-density oligonucleotide arrays. The previously identified Colo-specific palindrome at CTSK was used as a positive internal control, and pairwise comparisons between Colo and HDF revealed a robust positive signal within approximately 300 by of the known junction of the palindromic arm and non-palindromic spacer (FIG. 10A). Another previously confirmed DNA palindrome at the ECM1 locus (Tanaka et al., Nat. Genet. 37:320-327 (2005)) also showed a strong GAPF-positive signal on the tiling array (FIG. 10B), demonstrating that GAPF applied to whole-genome tiling arrays can accurately detect and map palindromic rearrangements.

When the GAPF data from the Colo and HDF cells was analyzed on a genome-wide scale, 120 GAPF-positive regions (Colo>HDF; log₂(signal ratio)>1.5; p<0.001; >100 kb between signals; filtered for c-MYC double minute amplification signal) were identified. Using these same statistical criteria, 9 GAPF-negative signals (i.e., HDF>Colo) were identified. These data support the above initial studies that GAPF-positive signals are more prevalent in cancer cells compared to normal cells. To verify that these newly identified GAPF-positive regions contained palindromes, a subset of these signals were chosen for analysis by Southern. Even though these loci were consistently identified as GAPF-positive in independent experiments, evidence was not found for DNA palindrome formation or genomic rearrangement at these loci.

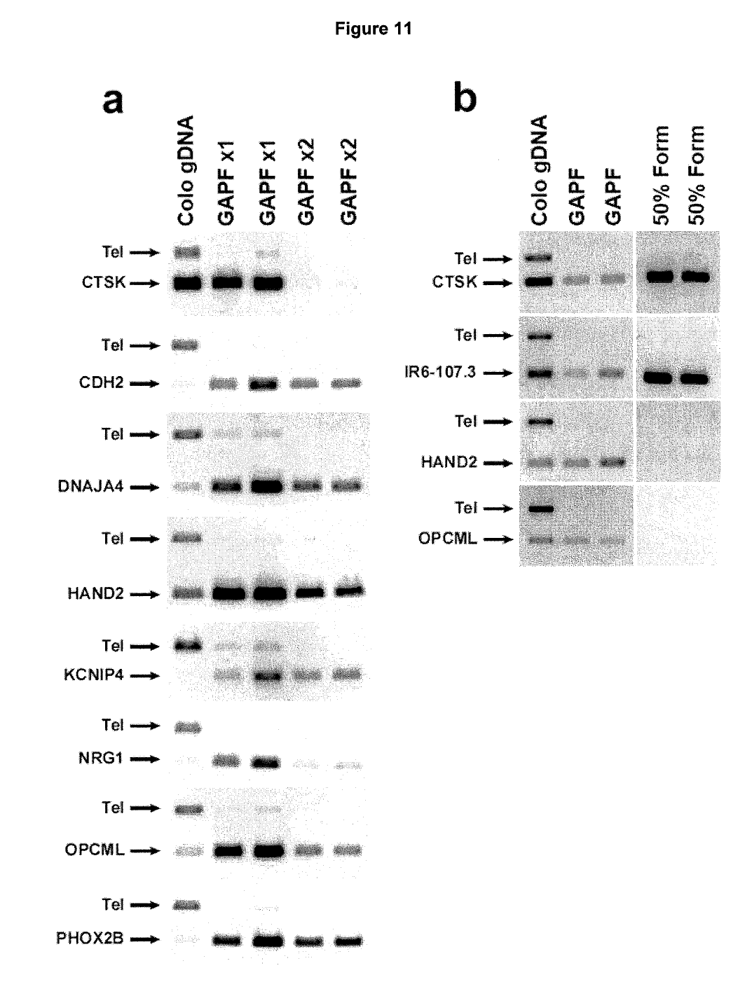

The nonpalindromic signals identified by GAPF were postulated to be due to regions of incomplete denaturation of genomic DNA that would remain S1 nuclease resistant. To initially test this possibility, a ‘cycled’ GAPF was performed in which a second cycle of denaturation/renaturation/S1-digestion after the initial round of GAPF was repeated. DNA regions resistant to denaturation during the first round of GAPF should also survive a second round of GAPF, whereas palindromic DNA would not survive the second round of GAPF because the loop of DNA holding two palindromic arms together would be digested by S1 in the first round of GAPF. Indeed, the palindromic region at the CTSK locus was enriched after the first round of GAPF in Colo cells but did not survive a second round of GAPF. Interestingly, the seven other loci examined that had reproducibly scored as GAPF-positive, but without evidence of palindrome formation (CDH2, DNAJA4, HAND2, KCNIP4, NRG1, OPCML and PHOX2B), survived the second round of GAPF, implying that the DNA at these loci were resistant to denaturation and/or S1 digestion (FIG. 11A).

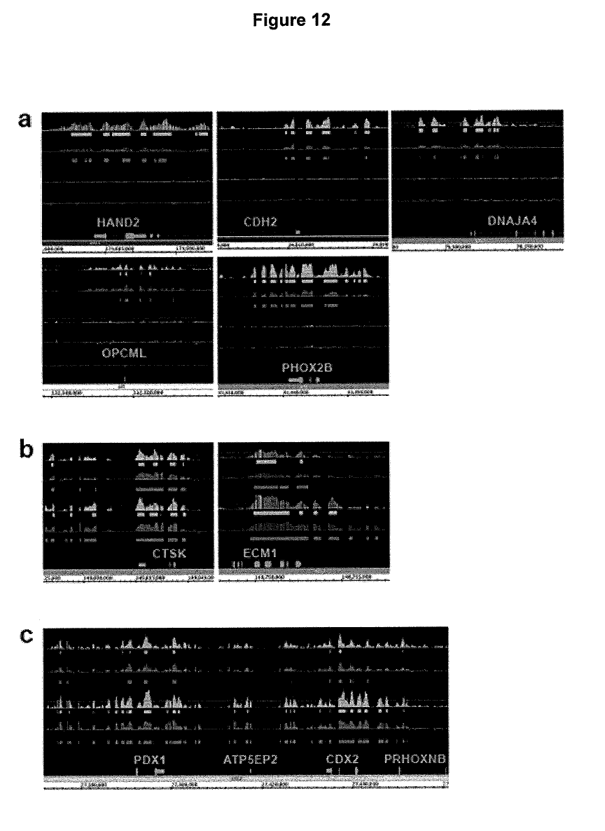

To directly determine whether the nonpalindromic GAPF-positive signals represented regions of incomplete DNA denaturation, formamide was added as a DNA helix destabilizer during the DNA denaturation step of the assay. Previous studies have shown that for every 1% of formamide, the DNA melting temperature (Tₘ) is reduced by 0.6-0.72° C. (Hutton, Nucleic Acids Research 4:3537-3555 (1977); McConaughy et al., Biochemistry 8:3289-3295 (1969)). Earlier experiments had also demonstrated that S1 nuclease is active in up to 60% formamide (Hutton & Wetmur, Biochem. Biophys. Res. Commun. 66:942-948 (1975). Therefore, a modified GAPF protocol was created by adding 50% formamide to the denaturation step, thus decreasing the Tₘ by about 35° C. A semi-quantitative PCR assay was used to analyze the GAPF-enrichment of two known DNA palindromes and two regions that were GAPF-positive using the original assay but were not in palindromic regions. Compared to the original GAPF procedure, the addition of 50% formamide greatly reduced the GAPF-positive signals generated by the nonpalindromic loci, whereas the GAPF-positive signals at previously identified palindromes, the CTSK locus and a naturally occurring DNA inverted repeat located on chromosome VI (Warburton et al., Genome Res. 14:1861-1869 (2004), were retained and somewhat enhanced (FIG. 11B and FIG. 12A). Thus, the lowering of the Tₘ by formamide eliminated GAPF-positive signals from non-palindromic regions of DNA, consistent with the hypothesis that these were caused by incomplete denaturation.

Whole genome analysis using the formamide-modified GAPF procedure identified 16 GAPF-positive regions, compared to the 120 GAPF-positive regions using the original protocol without formamide, and 8 GAPF-negative regions, compared to 9 previously. The GAPF-positive tiling array signals at loci with validated DNA palindromes, such as CTSK and ECM1 were enhanced by formamide-modified GAPF (FIG. 12B). A genomic region spanning approximately 170 kb on chromosome 13 also became more pronounced (FIG. 12C), which was a new potential DNA palindrome detected using GAPF-palindrome bordering a region of genomic amplification in Colo320DM cells. Interestingly, this region has previously been shown to be amplified in Colo320DM cells by a CGH analysis (Barrett et al., Proc. Natl. Acad. Sci. USA 101:17765-17770 (2004)). Similar to the CTSK locus, it was possible that a DNA palindrome defines the borders of the amplicons in this region and thus was enriched in the GAPF assay. In summary, the formamide-modified GAPF procedure enhanced detection of palindromes and eliminated most of the non-palindromic signals. GAPF can be used to identify regions of the genome susceptible to palindrome formation and to help understand mechanistically how gene amplification occurs in cancer (Tanaka & Yao, Nat. Rev. Cancer 9:216-224 (2009)).

The elimination of the majority of the non-palindromic signals by the addition of formamide to the original GAPF procedure indicated that these signals were secondary to incomplete DNA denaturation in the Colo DNA sample compared to the control sample. Southern and sequencing analysis did not identify primary sequence or structural differences between samples at these loci (data not shown), and therefore it was concluded that cell-specific epigenetic modification was increasing the DNA denaturation temperature at these regions in the Colo cells.

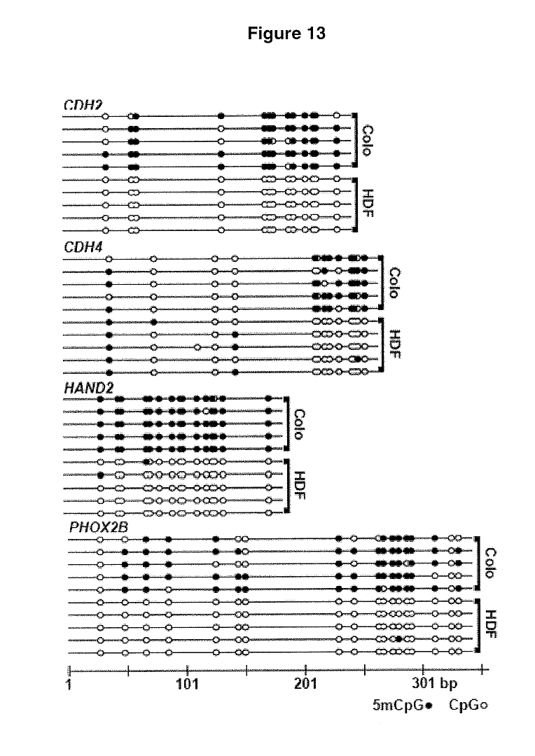

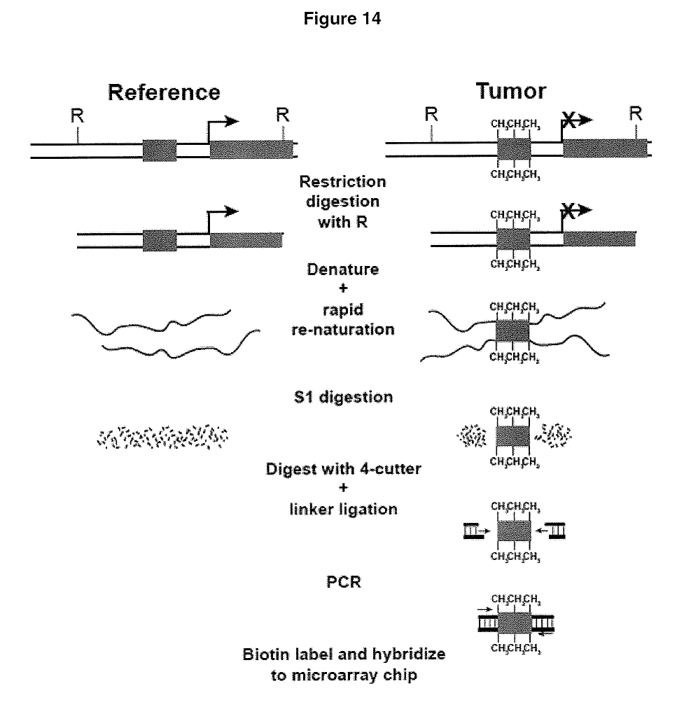

CpG DNA methylation is an epigenetic modification that has been shown to increase the Tₘ of DNA (Ehrlich et al., Biochim. Biophys. Acta, 395:109-119 (1975); Gill et al., Biochim. Biophys. Acta, 335:330-348 (1974)). The methylation status of a subset of the nonpalindromic GAPF-positive loci was initially assessed by the methylation sensitive restriction endonuclease HpaII or its methylation-insensitive isoschizomer MspI. While this assay only interrogates the methylation status of one CpG dinucleotide in the recognition sequence of the enzyme (CCGG), it was interesting to find that most of these loci showed more methylation in Colo cells than HDF cells (Table 2). To confirm that the GAPF-positive non-palindromic loci were indeed differentially methylated in Colo cells, bisulfite DNA sequence analysis of four selected loci was performed. Strikingly, all of these loci showed heavy DNA methylation in Colo cells compared to the HDF controls (FIG. 13). Thus, the non-palindromic GAPF-positive signals observed in cancer cells represented regions of differential methylation that altered the Tₘ of DNA denaturation.

TABLE 2
Methylation status of nonpalindromic loci.

Methylation status

| Locus | Colo320DM | HDF |
|---|---|---|
| CDH2 | + | − |
| CDH4 | − | − |
| DNAJA4 | + | +/− |
| GDF6 | + | +/− |
| HAND2 | + | − |
| KCNIP4 | + | − |
| NRG1 | + | − |
| OPCML | − | − |
| PHOX2B | + | − |
| SCXB | + | − |
| TCF15 | + | + |
| VAV3 | + | − |
| VWA1 | + | + |
| ZNF521 | + | − |

Methylation status was determined by digesting genomic DNA with either HpaII or MspI, and then performing PCR for each locus. Primers for each locus flank the recognition site (CCGG) such that the generation of a PCR product off of HpaII digested genomic DNA indicates CpG methylation. A plus sign (+) in Table 2 represents PCR product generation, (+/−)<(+), and (−) no product observed. In each case MspI digested DNA gave no PCR product.

Given that the original GAPF protocol also identified regions of differential CpG DNA methylation, this original protocol can be generally referred to as MADD (Methylation Analysis by Differential Denaturation) when using this assay to detect CpG DNA methylation. It previously has been observed that cytosine methylation at the C-5 position increases the melting temperature of naked DNA (Ehrlich et al., Biochim. Biophys. Acta 395: 109-119 (1975); Gill et al., Biochim. Biophys. Acta 335:330-348 (1974)). It has been hypothesized that the increase in the stability of duplex DNA caused by cytosine methylation is a result of changes in base-base stacking interactions (Aradi, Biophys. Chem. 54:67-73 (1995)). This effect of methylated cytosine on duplex DNA has previously been used to detect methylation patterns of specific loci by using denaturing gradient gel electrophoresis (Collins & Myers, J. Mol. Biol. 198:737-744 (1987)), but this technique is not amenable to genome-wide studies. Differential denaturation can be used for genome wide studies and enriches for differential DNA methylation based on this increase in Tₘ caused by methylated cytosine. During the denaturation and rapid cooling steps described herein, conditions can be such that methylated DNA remains double stranded and S1-resistant, while an exact same sequence in a less methylated state can become single-stranded and hence digested by S1.

The following description provides exemplary methods and materials for conducting the present methods as described herein.

Genomic DNA was isolated from cells using the QIAGEN Blood and Cell Culture DNA Kit® per the manufacturer's protocol. A total of 2 μg of genomic DNA was used as starting material for the assay. The sample was split into two tubes such that 1 μg was digested with KpnI (10 Units, NEB™) and 1 μg was digested with SbfI (10 Units, NEB™) for at least 8 hours in a total volume of 20 μl for each digestion. The restriction enzymes were then heat inactivated at 65° C. for 20 minutes. The KpnI and SbfI digests were combined, and then split evenly into two tubes. To the 20 μl of the DNA mixture, 27.36 μl of water and 1.64 μl of 3M NaCl was added such that the final concentration of NaCl was 100 mM and the total volume was 49 μl. For the formamide variation of the protocol to more specifically enrich for DNA palindromes, formamide was added to a final concentration of 50% before DNA denaturing. Denaturation was performed by boiling samples in a water bath for 7 minutes followed by rapid renaturation by immersing samples in an ice-water bath for at least 3 minutes. S1 nuclease (Invitrogen™) digestion was performed by adding 6 μl 10× S1 nuclease buffer, 4 μl 3M NaCl, and 1 μl of S1 nuclease (diluted to 100 Units/μl using S1 Dilution buffer). Samples were then incubated for 60 minutes at 37° C. S1 was inactivated by extraction with phenol followed by a phenol:chloroform extraction. DNA was ethanol precipitated in the presence of 20 μg of glycogen, and the DNA pellet was resuspended in 80 μl of 1/10 TE. The sample was then divided evenly into two tubes, with one tube subjected to digestion with MseI (40 Units, NEB™) and the other tube with MspI (40 Units, NEB™) for at least 6 hours at 37° C. (final volume of each digestion was 50 μl). Restriction enzymes were subsequently heat inactivated at 65° C. for 20 minutes. For ligation-mediated PCR, linkers were first created by combining 100 μl of a 100 pmol/μl solution of each oligonucleotide with 6.9 μl of 3M NaCl (final concentration 100 mM) and boiling in a water bath for 7 minutes. The water bath was then allowed to slowly cool to 25° C. to allow for annealing. Linkers were recovered by ethanol precipitation and the DNA pellet was resuspended in 500 μl of water. For the MseI linker, JW-102 g (SEQ ID NO: 1) was annealed to JW103 pcTA (SEQ ID NO: 5). For the MspI linker, JW-102 g (SEQ ID NO: 1) was annealed to JW103 pc-2 (SEQ ID NO: 2). Linkers were then ligated onto the MseI or MspI digested DNA by adding 5 μl of the appropriate linker to the 50 μl digest, then 7 μl 10×T4 DNA ligase buffer, 1 μl T4 DNA ligase (400 Units, NEB™) and 7 μl water for a final volume of 70 μl. Ligation was performed at 16° C. for at least 8 hours and then heat inactivated at 65° C. for 10 minutes. Linkers were then removed using a YM-50 Microcon™ (Amicon™) filter by adding the 70 μl ligation mixture to the column followed by the addition of 160 μl of 1/10 TE. Columns were spun at 12000×g in a microcentrifuge for 5 minutes to almost dryness. 20 μl of 1/10 TE was then added to the membrane, incubated at room temperature for 5 minutes, and then the DNA was recovered by spinning at 1000×g for 3 minutes per the manufacturer's protocol. 4 μl of this DNA was used as template for PCR using the appropriate MseI (JW-102gMse (SEQ ID NO: 8)) or MspI (JW-102gMsp (SEQ ID NO: 6)) primer (4 μl DNA, 10 μl 10×PCR buffer, 10 μl 2 mM dNTPs, 20 μl 5×GC-rich solution, 12 μl primer (10 μmol/μl), 1 μl Taq, 43 μl water (reagents from ROCHE FastStart® Taq kit). PCR conditions were as follows: 96° C. 6 minutes, 30 cycles of 96° C. 30 seconds, 55° C. 30 seconds, 72° C. 30 seconds, with final extension of 72° C. for 7 minutes. MseI and MspI PCR products were combined and purified using a YM-30 Microcon™ (Amicon™) filter. The 200 μl of PCR reaction was placed on the column and 300 μl of 1/10 TE was added. The column was spun at 14000×g until sample was concentrated to approximately 25 μl, and DNA was recovered into a new tube (1000×g for 3 minutes). DNA was quantitated and 7.5 μg of DNA was subjected to DNA fragmentation as follows: 44 μl DNA (7.5 μg total), 5 μl 10×DNase I buffer, 1 μl DNase I (diluted to 0.017 Units in water, NEB™) for 25 minutes at 37° C. with subsequent heat inactivation at 95° C. for 15 minutes. Fragmented DNA was labeled with biotin for hybridization on Affymetrix™ Human Tiling Arrays using the Affymetrix™ GeneChip® Whole-Transcript Double-Stranded Target Kit. To 45 μl of the fragmented DNA (6.75 μg DNA) from the previous step, 12 μl 5×TdT buffer, 2 μl TdT and 1 μl DNA labeling reagent were added, incubated at 37° C. for 60 minutes, and then heat inactivated at 70° C. for 10 minutes. Samples were processed per the manufacturer's protocol.

PCR-based enrichment assay. The assay was performed as described above through the DNA precipitation step after the inactivation of 51 nuclease with the modification that the DNA pellet was resuspended in 100 μl of 1/10 TE rather than 80 μl. 5 μl of this DNA was used in a PCR as follows: 5 μl template DNA, 5 μl 10×PCR buffer, 5 μl 2 mM dNTPs, 10 μl 5×GC-rich solution, 4 μl Tel F+R primer mix (5 pmol/μl of each), 4 μl F+R primer mix to region of interest (5 pmol/μl each), 0.4 μl Taq, 16.6 μl water (reagents from ROCHE FastStart® Taq kit). PCR conditions were as follows: 96° C. 6 minutes, 30 cycles of 96° C. 30 seconds, 58° C. 30 seconds, 72° C. 45 seconds, with final extension of 72° C. for 7 minutes.

| Primers. Tel | |
|---|---|
| (SEQ ID NO: 11 | |
| (Forward: | CTCCTCAGTCCCCTATGACTACATTT; |
| (SEQ ID NO: 12)) | |
| Reverse: | GCCCAGCCAATATACAACTGTAAAGC, |
| CTSK2 | |
| (SEQ ID NO: 13) | |
| (Forward: | GTCTAGGGCTCCTGCTCCTT; |
| (SEQ ID NO: 14)) | |
| Reverse: | GCAGGAGCTTTGGAATTACG, |
| mCDH2 | |
| (SEQ ID NO: 15 | |
| (Forward: | CCGGAGGGAAGCCTAGAGT; |
| (SEQ ID NO: 16)) | |
| Reverse: | GGCTGTTCCAGTACATCCTCA, |
| mCDH4 | |
| (SEQ ID NO: 17 | |
| (Forward: | GCAGAC ACTCCTGACAGCTC; |
| (SEQ ID NO:18)) | |
| Reverse: | CGGTCTTAGTCCGACTTCC, |
| mDNAJA4 | |
| (SEQ ID NO: 19) | |
| (Forward: | AGCCCATTCATTCCTCCATT; |
| Reverse: | CGCTTTTATCA GGTAGGCAGT, |
| mGDF6 | |
| (SEQ ID NO: 20) | |
| (Forward: | CACGACTCCACCACCATGT; |
| (SEQ ID NO: 21)) | |
| Reverse: | CTACGCTGCAGCAAGAAGC, |
| mHAND2 | |
| (SEQ ID NO: 22) | |
| (Forward: | AGCCCGATCTGGGTTCTT; |
| (SEQ ID NO: 23)) | |
| Reverse: | GAGAACCACCGCCGTCAC, |
| mKCNIP4 | |
| (SEQ ID NO: 24) | |
| (Forward: | TGCATAAACAACCTCGGAAA; |
| (SEQ ID NO: 25)) | |
| Reverse: | GCAGACCCGTGGACAGAC, |
| mNRG1 | |
| (SEQ ID NO: 26) | |
| (Forward: | AAGAAGGA CTCGCTGCTCAC; |
| (SEQ ID NO: 27)) | |
| Reverse: | CTCCAGTGGCAAAGCCTAAG, |
| mOPCML | |
| (SEQ ID NO: 28) | |
| (Forward: | GAGGGAAGGGGCAGAGTT; |
| (SEQ ID NO: 29)) | |
| Reverse: | TGACAGCTCCTGTATGTCAGAGA, |
| mPHOX2B | |
| (SEQ ID NO: 30) | |
| (Forward: | GAAGCAG GGGGAGAAAGAAG; |
| (SEQ ID NO: 31)) | |
| Reverse: | GCTCTTCCAGGCTCAAAGG, |
| mSCXB | |
| (SEQ ID NO: 32) | |
| (Forward: | CTGCACCTTCACATTTTCCA; |
| (SEQ ID NO: 33)) | |
| Reverse: | TTCTTGTGCTGTGTGGACCT, |
| mTCF15 | |
| (SEQ ID NO: 34) | |
| (Forward: | CAAACACCAG TAGTTCGTTCG; |
| (SEQ ID NO: 35)) | |
| Reverse: | CCTTTGGCTCAGCAATTCTC, |
| mVAV3 | |
| (SEQ ID NO: 36) | |
| Forward: | CCTAGTTGCCCCTAGTGGTG; |
| (SEQ ID NO:37)) | |
| Reverse: | GTTCTGGGGTCAAGTTCCAA, |
| mVWA1 | |
| (SEQ ID NO: 38) | |
| (Forward: | AACCTCCA CGTGGCCTTC; |
| (SEQ ID NO: 39)) | |
| Reverse: | CCTCACAACATGAGGAAGTGG, |
| mZNF521 | |
| (SEQ ID NO: 40) | |
| (Forward: | GCACAGGTATTTTGCAGTTCG; |
| (SEQ ID NO: 41)) | |
| Reverse: | GCGAAGTACCAGGACAAACC, |
| mCDH2s2 | |
| (SEQ ID NO: 42) | |
| (Forward: | AATTTAAT GGAGATGAAGAATGG; |
| (SEQ ID NO: 43)) | |
| Reverse: | TCAAACTCCCAAAAAAAACA, |
| mCDH4s1 | |
| (SEQ ID NO: 44) | |
| (Forward: | TTTTTAGTTTAGGTTAGGGT; |
| (SEQ ID NO: 45)) | |
| Reverse: | ACACCCTTTCTAAATAAAAC, |
| mHAND2as2 | |
| (SEQ ID NO: 46 | |
| (Forward: | ATCTCAATA CATCCATTTTCTCA; |
| (SEQ ID NO: 47)) | |
| Reverse: | GTTGTATATGGAGATTTTGT, |
| mPHOX2Bs1 | |
| (SEQ ID NO: 48) | |
| (Forward: | AGAAATTTTTTTAGGGGGAGT; |
| (SEQ ID NO: 49)) | |
| Reverse: | ACTTACTCCAACCTATTAAACA, |
| and | |
| PTCHl_bis | |
| (SEQ ID NO: 50) | |
| (Forward: | GAGGATTGTAGAAGAATATTA; |
| (SEQ ID NO: 51)) | |
| Reverse: | ACATTTAAATAACATA CCCC. |

Restriction enzyme-mediated methylation detection. Genomic DNA (1 μg) was digested with either MspI or HpaII (both from NEB™). This DNA (20 ng) was then used as template in a 30 cycle PCR (conditions as above) with primers that were designed to amplify across a recognition site for MspI/HpaII.

Bisulfite sequencing. Genomic DNA (1 μg) was treated with bisulfite per manufacturer's protocol (Qiagen™ EpiTect® Bisulfite Kit) and eluted in a total of 40 μl. PCR reaction: 4 μl DNA, 2.5 μl 10×PCR buffer, 2.5 μl 2 mM dNTPs, 2 μl. primer F+R mix (5 pmol/μl each), 5 μl 5×GC-rich solution, 0.2 μl Taq and 8.8 μl water (reagents from Roche™ FastStart® Taq Kit). PCR conditions: 96° C. 6 minutes, 5 cycles of 96° C. 45 seconds, 50° C. 90 seconds, 72° C. 2 minutes followed by 30 cycles of 96° C. 45 seconds, 50° C. 90 seconds, 72° C. 90 seconds followed by final extension of 72° C. for 7 minutes. PCR products were gel purified (QIAquick Gel Extraction Kit™, Qiagen™) and cloned (TOPO TA® Cloning Kit for Sequencing, Invitrogen™). Independent clones were isolated, plasmid DNA purified (QIAprep® Miniprep Kit, Qiagen™), and subjected to sequencing (Applied Biosystems™ 3730×1 DNA Analyzer per manufacturer's protocol). Sequence analysis was visualized using MethTools (Grunau et al., Nucl. Acids Res. 28:1053-1058, 2000).

Tiling Array Analysis. Affymetrix™ Human Tiling 2.0R Arrays and 1.0R Promoter Arrays were analyzed using Tiling Array Software (v 1.1.02, Affymetrix™). Raw data were scaled to a target intensity of 100 and normalized by quantile normalization. For probe analysis, a bandwidth of 250 by was used and perfect match (PM) probes were used in a Wilcoxon Rank Sum two-sided test. Two independent replicates were used for sample and control unless otherwise stated. Signal and p-value thresholds are stated for each experiment. For all experiments, a maximum gap of ≦100 and minimum run of >30 by were used. Data were visualized using the Integrated Genome Database Browser (v 5.12, Affymetrix™). For the generation of gene lists, .bed files generated in the above analysis were imported into NimbleScan® software (v 2.4), and a gene was denoted as positive if the GAPF-positive region mapped to −7 kb to +1.5 kb of the transcriptional start site.

### Example 4

The following example demonstrates the identification of methylated genomic loci in the colon cancer cell line HCT116 as compared to a derivative cell line having a disruption of the methylase enzymes DNMT1 and DNMT3b (DKO).

To determine whether a differential denaturation protocol can effectively be used to identify regions of differential DNA methylation genome-wide, the signal obtained using the assay above from the colorectal cancer cell line HCT116 was compared to its double DNA methyltransferase knockout (DKO) derivative that was generated by disrupting DNMT1 and DNMT3b, reducing global DNA methylation approximately 95% (Rhee et al., Nature 416:552-556 (2002)). The DKO derivative shares the same palindromes with the parental HCT116 cell line and as such there was no difference in the signal obtained for each cell line in the assay. As such, the only differences in signal were in the regions of DNA having differences in methylation. Further, since the initial focus was on the promoter CpG DNA hypermethylation found in cancer cells, the Affymetrix™ GeneChip® Human Promoter 1.0R Array was used to interrogate a subset of the genome consisting of >25,500 promoter regions with an average coverage from −7.5 to +2.45 kb relative to the transcriptional start site. Methylation-positive signals (log₂(signal ratio)>1.2 and p<0.001) were obtained that corresponded to the promoter regions of 563 genes (Table 3). When the same statistical criteria were used, no negative signal (DKO>HCT116) regions were identified.

TABLE 3
In one example, 563 genes resulted in methylation-positive signals
(HCT116 > DKO).

ABCB4
ABCC8
ABHD1
ACAA2
ACCN1
ACOT12
ACR
ACSS1
ACTC1
ACVRL1
ADAM12
ADAMTS18
ADAMTS19
ADAMTS2
ADAMTSL3
ADCYAP1R1
ADD2
ADRA2A
AFAP1L2
AK5
AKAP5
ALDH1A2
ALPK3
ALPL
ALX3
ALX4
AMIGO1
AMPH
ANKAR
ANKRD27
ANKRD38
AP1G2
APOB
ARHGAP20
ARHGAP27
ARL10
ARNT2
ARRDC4
ATP1A3
ATP6V1C2
ATRNL1
AVP
B4GALT4
BAALC
BARX2
BASP1
BCL11B
BHLHB5
BMP6
BMP7
C12orf53
C13orf21
C14orf2
C18orf34
C1orf164
C1orf59
C1orf76
C1orf95
C1QL2
C20orf177
C20orf39
C20orf58
C21orf70
C2orf40
C4orf19
C6orf60
C6orf97
CACNA2D1
CACNA2D3
CACNG2
CASD1
CBLN1
CBS
CCDC62
CCDC67
CCM2
CCND2
CDH22
CDH23
CDK5R2
CDX1
CECR6
CELSR3
CFC1
CGNL1
CGREF1
CHN2
CHRNA3
CHST1
CHST10
CHST11
CHST2
CITED2
CLDN11
CLSTN2
CNTN4
COL11A2
COL15A1
COL19A1
COL4A1
COL4A2
COL5A1
CPEB1
CPM
CPNE9
CPT1B
CPXM2
CRHR1
CRTAC1
CSMD2
CTNNA2
CTSF
CXCL12
CYP26A1
D4S234E
DAAM2
DBX2
DEGS2
DGKZ
DKFZP566E164
DLK1
DLL1
DLX3
DLX6
DMGDH
DMN
DMRT2
DMRT3
DMRTA2
DMRTB1
DOCK10
DPP10
DPP6
DPYSL5
DRD4
DSCAML1
DSCR6
DTX4
DUSP22
EBF1
ECE2
EDEM2
EDIL3
EFEMP2
EFHD1
EFS
EGR2
ELMOD1
EMILIN2
EML2
EMX1
EPHA4
EPHA6
ERC2
ERG
ERICH1
EVX1
FAM131B
FAM132A
FAM19A4
FAM20A
FAM26F
FAM43B
FAM78B
FAM98C
FANK1
FBLN2
FBLN5
FBN1
FBN2
FBXL21
FBXO17
FEZ1
FEZF2
FGD1
FGF4
FGF8
FIGN
FLJ33790
FLJ37440
FLJ44815
FLJ45717
FLT1
FMN2
FMNL3
FNDC4
FOXA2
FOXC2
FOXD3
FOXE1
FOXF1
FOXL1
FRAT1
FRAT2
FSTL4
FZD7
FZD9
GALC
GALNT14
GALNTL1
GAS1
GATA5
GATA6
GCKR
GDF10
GDF6
GDNF
GFRA2
GFRA4
GGN
GIPC3
GJB6
GLB1L3
GLDC
GLIS1
GLRB
GLT25D2
GNAL
GNG4
GPR25
GPR62
GPRIN2
GPT
GRASP
GREM1
GRIA2
GRM8
GSC
GUCY2D
GYG1
HCN4
HEPN1
HES5
HEY2
HHIP
HIST1H4K
HMBOX1
HNT
HOM-
TES-103
HOXA1
HOXA2
HOXB2
HOXB4
HOXC12
HOXC13
HOXD1
HOXD12
HOXD13
HOXD8
HOXD9
HS3ST2
HSF5
HTRA1
HTRA3
HTRA4
HYOU1
ID3
ID4
IGFBP4
IGFBP7
IGSF21
IL12RB2
IL13
IL17RC
INA
INHA
INPPL1
IRF4
IRX3
IRX4
ISL2
ITPKB
KCNA2
KCNA3
KCNA4
KCNB2
KCNC1
KCNF1
KCNG3
KCNH2
KCNIP1
KCNK10
KCNK12
KCNK4
KCNMB3
KCNN1
KCNQ3
KCNQ5
KCNS2
KCTD12
KIAA1024
KIAA1026
KIAA1191
KIAA1614
KIF7
KLHDC7B
KLHL14
KRBA1
LAMA1
LBH
LBX1
LBXCOR1
LEF1
LGI2
LHFPL4
LHX2
LHX3
LIF
LIMD2
LIMS2
LMO1
LOC253970
LOC285016
LOC390688
LOC400451
LOR
LRFN5
LRIG1
LRP12
LRRC24
LRRN1
LRRTM1
LYL1
MAL2
MAP6
MATN3
MEST
MFSD4
MFSD7
MGC33846
MGC4655
MGC70857
MGMT
MLC1
MLLT3
MMP2
MMP21
MOV10L1
MOXD1
MTNR1A
MYH11
NAT14
NCAM2
NDRG4
NEFH
NELL1
NEURL
NEUROG2
NFASC
NFE2L3
NFIB
NKX2-2
NKX2-4
NKX3-2
NKX6-1
NOVA1
NPAS1
NPB
NPL
NPR2
NPTX1
NPTX2
NR4A3
NRCAM
NRG2
NRIP3
NRXN1
NRXN2
NSD1
NTNG1
NUDT3
NUTF2
NXPH3
OLIG2
OPRD1
OPRK1
OTX2
OXTR
P2RX2
PALM2-
AKAP2
PAQR4
PARD3B
PAX1
PCDH7
PCSK1N
PDE4D
PDGFC
PDLIM4
PDZRN3
PER3
PFKFB3
PGR
PHOX2A
PHYHIPL
PIF1
PIP5K1B
PLCB1
PLXDC1
PLXNA2
POU2F3
POU3F2
PPM1E
PRCD
PRKACG
PRKD1
PROK2
PRR16
PRR18
PRTFDC1
PTF1A
PTGER3
PTHLH
PTPRB
PTPRZ1
PUNC
PXDN
RAMP1
RAMP2
RAPGEFL1
RASL10B
RASSF5
RBP4
REEP2
RFTN1
RGS20
RGS7
RHBDL1
RNF180
RORA
RORC
RPRML
RSPO3
RSPO4
RTN1
RYR3
SALL1
SAMD14
SARM1
SCGB1C1
SCGB3A1
SCT
SCTR
SCUBE1
SCUBE3
SELV
SEMA5A
SEMA6D
SEZ6
SEZ6L
SFMBT2
SFRP1
SFRP5
SGPP2
SH3GL3
SH3MD4
SH3PXD2A
SHE
SIX2
SIX3
SIX6
SKAP1
SLC10A4
SLC15A3
SLC16A12
SLC17A7
SLC18A3
SLC1A4
SLC22A3
SLC26A1
SLC32A1
SLC35D3
SLC39A7
SLC40A1
SLC6A1
SLC6A11
SLC6A20
SLC7A10
SLC8A3
SLC9A3
SLIT3
SMO
SMPD3
SNTB1
SORBS3
SORCS3
SOX1
SOX5
SOX7
SOX9
SP8
SPG20
SPOCK1
SPSB4
SSTR4
ST5
ST6GALNAC3WNT11
ST8SIA2
STAT5A
STX16
STXBP6
SUSD4
TAC4
TACC2
TAL1
TBX21
TBX4
TDRD10
TFAP2B
TFAP2E
THNSL2
THOC5
TIAM1
TIMP3
TJP2
TMEM130
TMEM132E
TMEM163
TMEM16B
TMEM178
TMEM179
TNFAIP8
TNFRSF1B
TNS3
TP53INP1
TPM4
TPPP3
TRIM58
TRIM71
TRIM73
TRIM74
TRPM2
TRPV4
TSPAN2
TSPAN31
TTLL9
UCHL1
USP51
UTF1
UTS2R
VAMP5
VASH2
VIPR2
VLDLR
VSTM2A
VWC2
WBSCR17
WIPF1
WNK2
WNT5A
WNT9B
WT1
XKR6
YBX2
YPEL3
ZFP36L2
ZFP37
ZNF141
ZNF184
ZNF22
ZNF503
ZNF642
ZNF703

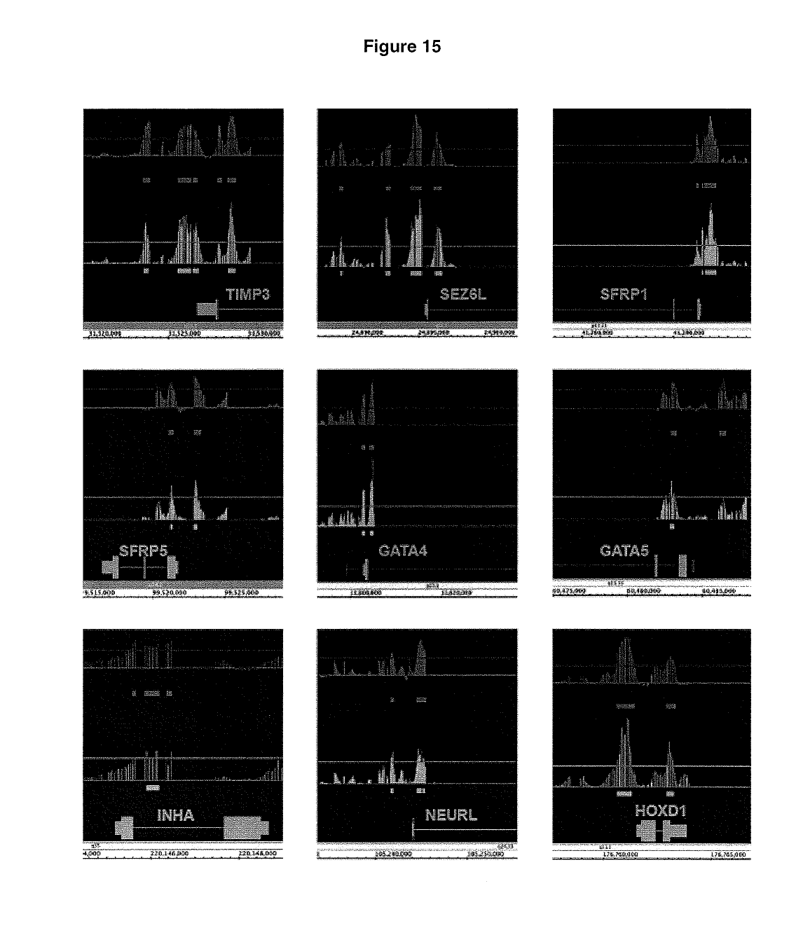

Methylation-positive signals (HCT116>DKO) showed a strong positive correlation with regions in HCT116 previously shown to be hypermethylated relative to the DKO line. The TIMP3 gene has been previously identified as methylated in HCT116 cells and unmethylated in DKO cells (Rhee et al., Nature 416:552-556 (2002), and the TIMP3 was found to be positive in the region of the promoter (FIG. 15). In addition, a number of other loci known to be methylated in HCT116 cells were also positive, such as SEZ6L (Suzuki et al., Nat. Genet. 31:141-149 (2002)), SFRP1 (Suzuki et al., Nat. Genet. 31:141-149 (2002)), SFRP5 (Suzuki et al., Nat. Genet. 31:141-149 (2002)), GATA4 (Akiyama et al., Mol. Cell. Biol. 23:8429-8439 (2003)), GATA5 (Akiyama et al., Mol. Cell. Biol. 23:8429-8439 (2003)), INHIBINα (Akiyama et al., Mol. Cell. Biol. 23:8429-8439 (2003)), NEURL (Schuebel et al., PLoS Genet. 3:1709-1723 (2007)), and HOXD1 (Schuebel et al., PLoS Genet. 3:1709-1723 (2007); Jacinto et al., Cancer Res. 67: 11481-11486 (2007)) (FIG. 15). Therefore, the above described assay and its variations can be used to identify differentially methylated loci in genome-wide screens.

### Example 5

The following example provides an analysis of DNA having different CpG density and methylation. In this comparison the genes identified as having a methylation-positive signal when denatured without formamide were compared with the genes identified as having a methylation-positive signal when denatured with 0.5% formamide.

Because the melting temperature of DNA is a function of the CpG density and methylation, it was predicted that additional differentially methylated regions could be identified by varying the denaturation conditions. The denaturation step was therefore modified by adding 0.5% formamide and the differential denaturation repeated in HCT116 and DKO cells. Positive signals were obtained in the promoter region of 455 genes, 241 of which were not identified using the original denaturation conditions above (Table 4). Some of these 241 positives have been previously characterized as being methylated in HCT116 cells compared to DKO cells, such as HIC1 (Arnold et al., Int. J. Cancer 106:66-73 (2003)), CHFR (Toyota et al., Proc. Natl. Acad. Sci. USA 100:7818-7823 (2003)), and RASGRF2 (Jacinto et al., Cancer Res. 67: 11481-11486 (2007)). Thus, the total number of unique positive promoter regions identified with these two denaturation conditions encompasses 804 genes, a substantially larger number than identified using the MeDIP assay (methylated DNA immunoprecipitation) in HCT116 (Jacinto et al., Cancer Res. 67: 11481-11486 (2007)). One hundred and twenty-six candidate hypermethylated genes in HCT116 versus DKO were identified in the MeDIP study (Jacinto et al., Cancer Res. 67: 11481-11486 (2007)), with only 7 of these genes (ERG1, FANK1, HOXD1, RASGRF2, RORC, ZNF141 and ZSCAN1) overlapping with the differential denaturation data set. This suggests that the present differential denaturation assay, under the conditions used herein, identified a largely distinct set of methylated regions compared to MeDIP.

TABLE 4
Methylation-positive signals resulted for an additional 241 genes that did
not show up in original denaturation conditions, which did not include
0.5% formamide.

ACHE
ACTN2
ADAMTS8
ADRA1D
ADRB1
AFF3
AIFM3
APCDD1
ARHGDIG
ARTN
ASTN2
ATOH7
ATP10A
AUTS2
B3GALT6
B3GAT1
BAD
BAI3
BARHL2
BEGAIN
BHLHB4
BMP2
BMP8A
BMP8B
BMPR1B
BNC1
BRUNOL4
C10orf25
C1orf69
C1QL1
C9orf4
CACNG7
CAMK2N2
CDH2
CEBPA
CELSR1
CG018
CHFR
CHRDL2
CLIP4
COL23A1
COL27A1
COLEC12
CPAMD8
CRAMP1L
CRMP1
CUGBP2
CYGB
DACT3
DCHS1
DCLK1
DGKI
DIO3
DLGAP4
DRD2
DTNA
ECEL1
EMILIN3
EN1
EN2
EPB41L3
EVC
EVC2
EVX2
EXOC3L2
FAM123C
FAM49A
FBXL11
FBXL7
FEV
FLJ45557
FLNC
FN3K
FNDC1
FOXC1
FOXD2
FOXE3
FOXG1
FOXL2
FZD10
GABRG3
GDF1
GLT1D1
GNAO1
GPR101
GPR150
GPR68
GPR88
GPX7
GRIK3
GRIN1
GRIN2C
GRIN3B
GRM6
GUCY1A2
GUCY1A3
HCN1
HDGFRP3
HERC2
HIC1
HOMER2
HOXB3
HRH3
HS3ST6
HS6ST3
HSPA12B
HUNK
IFT172
IGF2
IGF2AS
IGFBPL1
IHH
INSM1
IRS1
IRX5
ITGA9
KCNB1
KCND2
KCND3
KCNIP4
KCNK3
KCNK9
KCNMA1
KCTD21
KIAA1045
KIF1A
KIF26A
LASS1
LENG9
LOC164714
LOC285382
LOC389813
LOC401089
LOC91461
LRRC3B
MAFA
MAMDC4
MARCKS
MEIS2
MFSD3
MGAT5B
MIXL1
MLNR
MUPCDH
MYCN
NANOS1
NDN
NETO1
NETO2
NKX2-3
NLF1
NPAS3
NRG1
OLIG1
ONECUT1
ONECUT2
OPCML
PANK4
PCDH19
PCSK2
PDE8B
PELI2
PEX5L
PHOX2B
PHPT1
PID1
PKNOX2
PLD5
PLEC1
PODXL2
POLR2L
POU3F1
PPP1R3D
PRDM2
PRKCB1
PRTN3
PTPRM
PTPRT
PYGO1
RAB11FIP4
RAB42
RASGRF2
RASL10A
RBM32A
RBM32B
RELN
RET
RGMA
RGS11
RGS17
RIMS1
RPESP
RYR2
SCARF2
SCCPDH
SCUBE2
SCXB
SDF4
SHC3
SHROOM4
SLC16A8
SLC1A6
SLC24A3
SLC24A4
SLC4A4
SORCS2
SOX11
SOX21
SPRY2
STUB1
SULF2
SULT4A1
SYCE1
TBX2
TBX6
TCBA1
TCERG1L
TCF15
TCF4
THBS4
TLE4
TMEM47
TNFAIP2
TRPS1
TSHZ3
TUB
UBE2E2
UFSP1
UNCX
VAV3
VEGFC
VENTX
VGLL2
VPS13C
WIZ
WSCD1
ZAR1
ZDBF2
ZFP28
ZFPM2
ZSCAN1

Recently, a study identified CpG methylation in HCT116 cells using a genome-wide DNA methylation assay known as Methyl-seq (Brunner et al., Genome Res. published online on Mar. 9, 2009). Genomic DNA is first digested with either the methylation-sensitive restriction enzyme HpaII or its methylation-insensitive isoschizomer MspI, and then these fragment libraries are subjected to next-generation Solexa sequencing to determine CpG methylation status. When this publicly available dataset was analyzed to identify genes that have methylated CpG dinucleotides in their promoter regions, over 5500 genes are positive. Of these approximately 5500 genes identified, 84% (676/804) of the positive signal genes were represented. In contrast, of the 126 candidate hypermethylated genes in the MeDIP study of HCT116 (Jacinto et al., Cancer Res. 67: 11481-11486 (2007)), 15% (19/126) are identified using Methyl-seq. Thus, compared to MeDIP, the present assay identifies a substantially larger proportion of differentially methylated genes.

Since promoter hypermethylation has been associated with decreased gene expression, RNA expression levels were correlated with signal-positive regions. A publicly available dataset (GEO GSE11173) was used comparing the RNA expression level of DKO to HCT116 (McGarvey et al., Cancer Research 68: 5753-5759 (2008)). Out of the 804 signal-positive genes, 357 genes were represented on the array and had a statistically significant change in RNA expression level (p-value<0.05), of which, 301 (84%) of these genes had a higher level of RNA expression in DKO than HCT 116 (log₂(signal ratio)>0) (Table 5). These results further support the hypothesis that the majority of loci enriched by the present method identify regions of CpG hypermethylation.

TABLE 5
Expression levels of 357 genes compared between DKO and HCT116.

| | Log2SignalRatio | Pᵥₐₗᵤₑ |
|---|---|---|
| Gene Name | (DKO/HCT116) | Log2SignalRatio |
| HDGFRP3 | 2.46195 | 0.00000000 |
| NDN | 2.03393 | 0.00000000 |
| CHFR | 1.91729 | 0.00000000 |
| NPTX1 | 1.89611 | 0.00000000 |
| UCHL1 | 1.88238 | 0.00000000 |
| CXCL12 | 1.83868 | 0.00000000 |
| BNC1 | 1.7674 | 0.00000000 |
| C1orf59 | 1.66003 | 0.00000000 |
| ECEL1 | 1.59284 | 0.00000000 |
| SFRP1 | 1.59119 | 0.00000000 |
| NEFH | 1.58969 | 0.00000000 |
| TSPAN2 | 1.5789 | 0.00000000 |
| PRKCB1 | 1.48586 | 0.00000000 |
| NPTX2 | 1.47761 | 0.00000000 |
| TSHZ3 | 1.46734 | 0.00000000 |
| PXDN | 1.43132 | 0.00000000 |
| PRTFDC1 | 1.39522 | 0.00000000 |
| ATRNL1 | 1.37441 | 0.00000000 |
| PODXL2 | 1.35425 | 0.00000000 |
| AK5 | 1.33992 | 0.00000000 |
| FMNL3 | 1.3201 | 0.00000000 |
| SLC7A10 | 1.28862 | 0.00000000 |
| TUB | 1.28677 | 0.00000000 |
| VEGFC | 1.28496 | 0.00000000 |
| INA | 1.27519 | 0.00000000 |
| SYCE1 | 1.26451 | 0.00000000 |
| EVC2 | 1.26024 | 0.00000000 |
| COL4A2 | 1.25959 | 0.00000000 |
| HTRA3 | 1.25618 | 0.00000000 |
| ADD2 | 1.25109 | 0.00000000 |
| MAL2 | 1.22921 | 0.00000000 |
| CTSF | 1.22658 | 0.00000000 |
| ZFP28 | 1.21745 | 0.00000000 |
| IGFBP4 | 1.21241 | 0.00000000 |
| HOXD1 | 1.17765 | 0.00000000 |
| D4S234E | 1.14509 | 0.00000000 |
| COL4A1 | 1.12417 | 0.00000000 |
| DMRT2 | 1.10391 | 0.00000000 |
| PGR | 1.10109 | 0.00000000 |
| LOC285382 | 1.09352 | 0.00000000 |
| ABCC8 | 1.09172 | 0.00000000 |
| HOM-TES-103 | 1.08984 | 0.00000000 |
| RAB42 | 1.08921 | 0.00000000 |
| HOXB2 | 1.07687 | 0.00000000 |
| LEF1 | 1.07257 | 0.00000000 |
| SLC40A1 | 1.05005 | 0.00000000 |
| FBLN2 | 1.04715 | 0.00000000 |
| CDH2 | 1.03182 | 0.00000000 |
| MOV10L1 | 1.03027 | 0.00000000 |
| BMP2 | 1.02554 | 0.00000001 |
| FIGN | 1.02195 | 0.00000001 |
| CBS | 1.02004 | 0.00000000 |
| GDF6 | 1.01976 | 0.00000000 |
| FLNC | 1.00786 | 0.00000000 |
| TBX2 | 1.00307 | 0.00000000 |
| LOC400451 | 1.0005 | 0.00000000 |
| MLC1 | 1.00049 | 0.00000000 |
| EFS | 0.99901 | 0.00000000 |
| ID4 | 0.997266 | 0.00000000 |
| TBX21 | 0.993409 | 0.00000000 |
| HSPA12B | 0.992986 | 0.00000000 |
| FNDC1 | 0.985385 | 0.00000000 |
| VENTX | 0.975648 | 0.00000005 |
| ACSS1 | 0.972752 | 0.00000000 |
| COLEC12 | 0.965921 | 0.00000009 |
| TBX4 | 0.961524 | 0.00000000 |
| SPG20 | 0.95577 | 0.00000002 |
| GDF10 | 0.947233 | 0.00000000 |
| SULF2 | 0.946875 | 0.00000000 |
| TIMP3 | 0.940942 | 0.00000000 |
| HOXC12 | 0.927776 | 0.00000083 |
| CGREF1 | 0.924638 | 0.00000000 |
| TCF15 | 0.905041 | 0.00000001 |
| PRKD1 | 0.883234 | 0.00000000 |
| HTRA1 | 0.882529 | 0.00000000 |
| NPL | 0.875609 | 0.00000000 |
| OLIG1 | 0.873375 | 0.00000163 |
| WT1 | 0.870339 | 0.00000000 |
| GATA5 | 0.867291 | 0.00000000 |
| LRIG1 | 0.861875 | 0.00000000 |
| NEURL | 0.858916 | 0.00000000 |
| LOC285016 | 0.853966 | 0.00000754 |
| KLHDC7B | 0.850641 | 0.00000000 |
| EVC | 0.847638 | 0.00000000 |
| TLE4 | 0.847023 | 0.00000101 |
| NRG2 | 0.836327 | 0.00002508 |
| DCHS1 | 0.830064 | 0.00000006 |
| FOXL2 | 0.822225 | 0.00000000 |
| RPRML | 0.819382 | 0.00000000 |
| LAMA1 | 0.801227 | 0.00011983 |
| TCF4 | 0.800773 | 0.00000000 |
| WNT5A | 0.787736 | 0.00006535 |
| PDLIM4 | 0.785976 | 0.00000000 |
| LBH | 0.78528 | 0.00000000 |
| STAT5A | 0.782296 | 0.00000000 |
| NKX2-3 | 0.777173 | 0.00024356 |
| HES5 | 0.773653 | 0.00000000 |
| FAM43B | 0.772604 | 0.00000000 |
| GRASP | 0.770645 | 0.00000000 |
| PER3 | 0.765325 | 0.00000000 |
| AMPH | 0.758622 | 0.00060387 |
| TMEM130 | 0.748795 | 0.00000138 |
| PKNOX2 | 0.744319 | 0.00069505 |
| RBP4 | 0.74138 | 0.00000000 |
| FEZ1 | 0.74104 | 0.00000000 |
| NETO2 | 0.739978 | 0.00000000 |
| SLC22A3 | 0.739132 | 0.00038851 |
| HEY2 | 0.730564 | 0.00067551 |
| GPR68 | 0.727836 | 0.00000001 |
| CDH22 | 0.725277 | 0.00000000 |
| GREM1 | 0.717244 | 0.00197168 |
| SH3PXD2A | 0.714474 | 0.00000000 |
| GALC | 0.713326 | 0.00200159 |
| CHST2 | 0.712975 | 0.00000000 |
| KCNC1 | 0.710469 | 0.00148113 |
| VAV3 | 0.708528 | 0.00000000 |
| PDE8B | 0.705531 | 0.00000143 |
| LOC91461 | 0.700719 | 0.00000000 |
| ADAMTS2 | 0.693602 | 0.00000000 |
| SNTB1 | 0.690754 | 0.00267747 |
| FOXE1 | 0.68733 | 0.00000000 |
| XKR6 | 0.685501 | 0.00191419 |
| KCTD12 | 0.683722 | 0.00000000 |
| HOMER2 | 0.679011 | 0.00000000 |
| KIAA1045 | 0.678605 | 0.00125537 |
| SLC32A1 | 0.67843 | 0.00000000 |
| HS6ST3 | 0.674754 | 0.00441248 |
| HOXD9 | 0.674416 | 0.00000000 |
| TRPS1 | 0.667854 | 0.00646714 |
| KIF7 | 0.664097 | 0.00000000 |
| SLC15A3 | 0.657256 | 0.00000000 |
| TPM4 | 0.653803 | 0.00000000 |
| IL12RB2 | 0.651874 | 0.00353941 |
| C6orf60 | 0.651108 | 0.00000000 |
| CDH23 | 0.648518 | 0.00010794 |
| GALNTL1 | 0.645102 | 0.00000000 |
| GSC | 0.640548 | 0.00000000 |
| KCNMB3 | 0.634232 | 0.01239150 |
| SOX7 | 0.625382 | 0.00894783 |
| TMEM16B | 0.621789 | 0.00000000 |
| CPM | 0.62032 | 0.00253372 |
| COL5A1 | 0.616956 | 0.01593390 |
| ATP10A | 0.614604 | 0.00000007 |
| GABRG3 | 0.611562 | 0.01068870 |
| CYP26A1 | 0.611283 | 0.00000000 |
| GPX7 | 0.605692 | 0.00000000 |
| HOXB4 | 0.604514 | 0.00000000 |
| TNFAIP2 | 0.602667 | 0.00000000 |
| RAMP1 | 0.6019 | 0.00000000 |
| SCCPDH | 0.588139 | 0.00000005 |
| FAM19A4 | 0.588125 | 0.00000000 |
| USP51 | 0.581825 | 0.00811829 |
| LIF | 0.576336 | 0.00000000 |
| IRX3 | 0.57277 | 0.00000000 |
| CACNA2D3 | 0.571324 | 0.00000353 |
| IGF2 | 0.569082 | 0.00000000 |
| FLT1 | 0.567601 | 0.00092879 |
| RASSF5 | 0.564 | 0.02925730 |
| GALNT14 | 0.557542 | 0.00000000 |
| NANOS1 | 0.557021 | 0.00000000 |
| IGFBPL1 | 0.551414 | 0.00000000 |
| NTNG1 | 0.549156 | 0.00046033 |
| HOXD13 | 0.547516 | 0.00000000 |
| CRMP1 | 0.546673 | 0.00000001 |
| CHRDL2 | 0.542375 | 0.03815240 |
| DLX6 | 0.538737 | 0.00968645 |
| TNFRSF1B | 0.537973 | 0.00000285 |
| NRIP3 | 0.536296 | 0.00000000 |
| LRFN5 | 0.532825 | 0.04098170 |
| SUSD4 | 0.531107 | 0.04997800 |
| PPM1E | 0.526075 | 0.04957330 |
| FBXL21 | 0.521842 | 0.00009906 |
| EN1 | 0.521353 | 0.00000692 |
| BASP1 | 0.520082 | 0.00000000 |
| IRX5 | 0.51409 | 0.00000002 |
| SMPD3 | 0.511393 | 0.00000000 |
| ADAMTSL3 | 0.506411 | 0.00000000 |
| TRIM58 | 0.504652 | 0.00000012 |
| CPAMD8 | 0.503399 | 0.00000000 |
| HOXC13 | 0.503319 | 0.00000000 |
| ALDH1A2 | 0.491275 | 0.02546600 |
| MGAT5B | 0.483148 | 0.01013510 |
| SPOCK1 | 0.482202 | 0.00026379 |
| DSCR6 | 0.478995 | 0.00000000 |
| FAM49A | 0.476307 | 0.00000001 |
| EFEMP2 | 0.474225 | 0.00000001 |
| SOX9 | 0.468049 | 0.00000000 |
| CACNA2D1 | 0.466351 | 0.00015036 |
| DMRT3 | 0.464338 | 0.00643338 |
| VLDLR | 0.454224 | 0.00213007 |
| WNT11 | 0.452474 | 0.00000000 |
| ABHD1 | 0.450293 | 0.00000229 |
| RGS11 | 0.443449 | 0.00000169 |
| KIF1A | 0.442234 | 0.00000002 |
| CCND2 | 0.440734 | 0.00101199 |
| GUCY2D | 0.43814 | 0.00000746 |
| AMIGO1 | 0.435686 | 0.03413680 |
| LHX2 | 0.430711 | 0.00000000 |
| C12orf53 | 0.416544 | 0.00677519 |
| PDE4D | 0.415289 | 0.01111750 |
| HOXB3 | 0.414088 | 0.00000003 |
| SLC24A3 | 0.413561 | 0.00563093 |
| LRP12 | 0.412747 | 0.00000009 |
| NFE2L3 | 0.40763 | 0.00000001 |
| DMN | 0.407168 | 0.00000016 |
| ZFP37 | 0.406256 | 0.00000000 |
| RET | 0.400774 | 0.00001873 |
| ST6GALNAC3 | 0.399489 | 0.00000020 |
| C13orf21 | 0.387835 | 0.00752451 |
| PUNC | 0.386145 | 0.00000144 |
| POLR2L | 0.382353 | 0.00000013 |
| TRPV4 | 0.379125 | 0.00024072 |
| KCNG3 | 0.378218 | 0.00000002 |
| FZD9 | 0.378163 | 0.00000021 |
| DLL1 | 0.378141 | 0.00000013 |
| SORCS2 | 0.377102 | 0.01096290 |
| SPRY2 | 0.370543 | 0.00000006 |
| ZNF141 | 0.364062 | 0.00001472 |
| C1QL1 | 0.357152 | 0.00010475 |
| CHST10 | 0.354426 | 0.00038934 |
| SULT4A1 | 0.352165 | 0.00000079 |
| SCUBE1 | 0.351607 | 0.00000387 |
| HIC1 | 0.350797 | 0.00388565 |
| NOVA1 | 0.336146 | 0.00324738 |
| FLJ33790 | 0.33205 | 0.00010206 |
| RAB11FIP4 | 0.327565 | 0.00090527 |
| FOXA2 | 0.326804 | 0.00319583 |
| VAMP5 | 0.323135 | 0.00001748 |
| CYGB | 0.316743 | 0.00683416 |
| BMP7 | 0.316065 | 0.00003135 |
| TJP2 | 0.315785 | 0.00001143 |
| NDRG4 | 0.315652 | 0.00000624 |
| C9orf4 | 0.307769 | 0.01988740 |
| SLC8A3 | 0.304421 | 0.00144858 |
| SLC10A4 | 0.297697 | 0.00003720 |
| ZAR1 | 0.293082 | 0.01212460 |
| STXBP6 | 0.291383 | 0.00003604 |
| EFHD1 | 0.288541 | 0.01278810 |
| ST5 | 0.286565 | 0.00009248 |
| NRXN2 | 0.282715 | 0.00010214 |
| ZFP36L2 | 0.282202 | 0.00009655 |
| DMRTB1 | 0.277942 | 0.00032007 |
| C4orf19 | 0.277766 | 0.02393910 |
| PLXNA2 | 0.272372 | 0.00063342 |
| CPEB1 | 0.27114 | 0.00115618 |
| MGC4655 | 0.271023 | 0.00011842 |
| CHST1 | 0.270797 | 0.00172692 |
| CHRNA3 | 0.266733 | 0.01787250 |
| PYGO1 | 0.264357 | 0.00073879 |
| COL27A1 | 0.263535 | 0.00013283 |
| GLIS1 | 0.261171 | 0.00025461 |
| NRG1 | 0.259443 | 0.00118161 |
| FOXF1 | 0.254161 | 0.00022717 |
| ONECUT1 | 0.248013 | 0.00157246 |
| DLX3 | 0.246384 | 0.00251122 |
| NSD1 | 0.2433 | 0.00013602 |
| DIO3 | 0.241827 | 0.00081988 |
| NR4A3 | 0.241251 | 0.00108803 |
| PHPT1 | 0.238017 | 0.00012666 |
| ZNF22 | 0.237665 | 0.00024548 |
| SLIT3 | 0.2369 | 0.00083076 |
| ARNT2 | 0.227284 | 0.00050780 |
| LHFPL4 | 0.223894 | 0.00155499 |
| LIMD2 | 0.220789 | 0.00429697 |
| FBXL11 | 0.219446 | 0.00089127 |
| FNDC4 | 0.218926 | 0.00385914 |
| NXPH3 | 0.218056 | 0.01195570 |
| PALM2-AKAP2 | 0.213459 | 0.00185401 |
| SH3GL3 | 0.212031 | 0.00051961 |
| PRDM2 | 0.210747 | 0.00274269 |
| CGNL1 | 0.209065 | 0.02980390 |
| CELSR3 | 0.203897 | 0.00258390 |
| PFKFB3 | 0.198755 | 0.01001570 |
| FOXC1 | 0.196785 | 0.00245823 |
| PPP1R3D | 0.196573 | 0.00403746 |
| ITGA9 | 0.196304 | 0.00159484 |
| IRX4 | 0.192808 | 0.00186541 |
| SCARF2 | 0.190571 | 0.00573162 |
| ALPL | 0.18549 | 0.02021600 |
| KCNN1 | 0.178805 | 0.02582290 |
| TRPM2 | 0.177768 | 0.00237912 |
| ZNF703 | 0.173716 | 0.01295460 |
| ECE2 | 0.171808 | 0.03700010 |
| DTNA | 0.171004 | 0.01075750 |
| IGF2AS | 0.168637 | 0.00850520 |
| B4GALT4 | 0.1681 | 0.01213510 |
| DRD4 | 0.166374 | 0.00759962 |
| MGC33846 | 0.165076 | 0.02021740 |
| SMO | 0.163824 | 0.01608620 |
| ASTN2 | 0.161321 | 0.01014720 |
| HERC2 | 0.155711 | 0.01557060 |
| LYL1 | 0.154795 | 0.01705970 |
| SIX2 | 0.153852 | 0.02030770 |
| ACCN1 | 0.150246 | 0.01717170 |
| DUSP22 | 0.143777 | 0.02538290 |
| BAD | 0.138326 | 0.03198800 |
| FOXD2 | 0.137433 | 0.02778640 |
| SLC17A7 | 0.136558 | 0.03756310 |
| MAMDC4 | 0.134781 | 0.04674780 |
| KIAA1191 | 0.127682 | 0.04828040 |
| GYG1 | 0.119033 | 0.04517560 |
| MGMT | −0.120489 | 0.04436320 |
| ACAA2 | −0.12613 | 0.04584530 |
| C1orf164 | −0.13618 | 0.02674980 |
| EMILIN2 | −0.141065 | 0.01774390 |
| PAQR4 | −0.141228 | 0.03518320 |
| INPPL1 | −0.142742 | 0.02843300 |
| YBX2 | −0.14287 | 0.03062350 |
| GLDC | −0.151476 | 0.01168670 |
| STX16 | −0.156361 | 0.01107110 |
| FBLN5 | −0.160205 | 0.02535800 |
| KIF26A | −0.185448 | 0.00715529 |
| HEPN1 | −0.188751 | 0.01701620 |
| UBE2E2 | −0.194483 | 0.00207548 |
| BMP8B | −0.200298 | 0.00220759 |
| ZNF184 | −0.201503 | 0.00381080 |
| ARTN | −0.214828 | 0.00170586 |
| RGS17 | −0.216088 | 0.00321792 |
| RORC | −0.220371 | 0.00233819 |
| VPS13C | −0.223244 | 0.00020525 |
| CASD1 | −0.224799 | 0.00113099 |
| B3GALT6 | −0.233049 | 0.00017464 |
| DLGAP4 | −0.234303 | 0.00027044 |
| HMBOX1 | −0.234421 | 0.00040242 |
| ARHGAP27 | −0.235535 | 0.00218529 |
| RGMA | −0.237438 | 0.00277914 |
| TACC2 | −0.241429 | 0.00041343 |
| NUDT3 | −0.242229 | 0.00024736 |
| CITED2 | −0.243734 | 0.00016286 |
| MFSD3 | −0.260001 | 0.00019352 |
| FZD7 | −0.263275 | 0.00007751 |
| GATA6 | −0.265431 | 0.00005529 |
| HOXA2 | −0.272077 | 0.01178820 |
| ATP6V1C2 | −0.278695 | 0.00001183 |
| EGR2 | −0.285223 | 0.00000848 |
| THBS4 | −0.293671 | 0.00018642 |
| TSPAN31 | −0.29535 | 0.00000325 |
| NPAS1 | −0.298 | 0.00006921 |
| TNS3 | −0.338476 | 0.00000152 |
| HIST1H4K | −0.354667 | 0.00000015 |
| CEBPA | −0.371838 | 0.00000013 |
| TNFAIP8 | −0.387377 | 0.00000018 |
| TRIM73 | −0.395493 | 0.00001903 |
| PLCB1 | −0.400098 | 0.00000002 |
| NFIB | −0.404334 | 0.00000020 |
| BCL11B | −0.431849 | 0.00000001 |
| GNAL | −0.459042 | 0.00065682 |
| FGF8 | −0.46457 | 0.00000879 |
| MEIS2 | −0.469743 | 0.00000181 |
| MLLT3 | −0.477841 | 0.00000001 |
| PCDH7 | −0.529917 | 0.00000070 |
| CG018 | −0.613001 | 0.00225287 |
| ARRDC4 | −0.698737 | 0.00000000 |
| IRS1 | −0.867164 | 0.00000000 |
| CCDC62 | −0.949383 | 0.00000000 |
| DMGDH | −1.08456 | 0.00000000 |
| MARCKS | −1.27182 | 0.00000000 |

### Example 6

The following example demonstrates the detection of CpG DNA methylation in primary medulloblastoma samples.

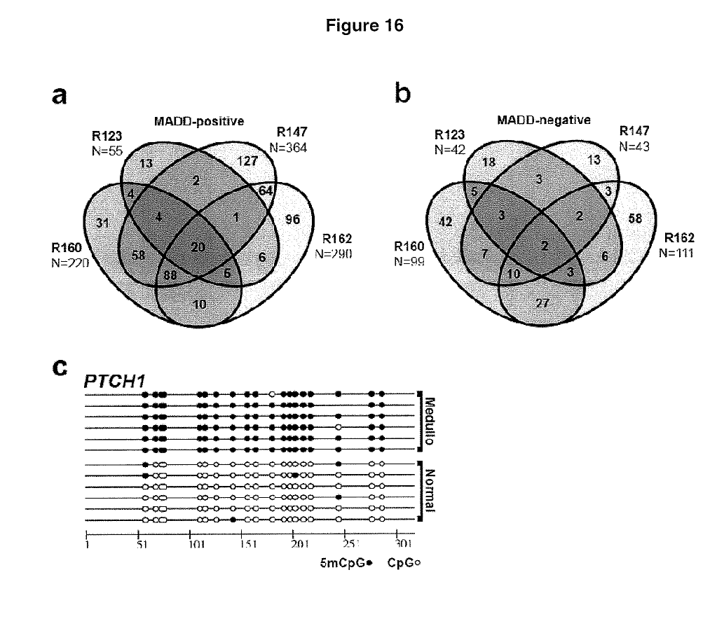

To test the hypothesis that present methods for enriching for methylated DNA can be used to identify cancer-specific methylation changes from patient samples, medulloblastoma biopsy specimens from four individual patients were analyzed using normal cerebellum as a control. In our previous study of DNA palindromes in cancer, common genomic regions between different medulloblastoma samples were found that scored as positive using the original palindrome assay (Tanaka et al., Nat. Genet. 37:320-327 (2005)). Given that the majority of signals from the assay have been found to be from differential DNA methylation, these regions were reexamined using a differential denaturation assay described above. Differential denaturation was performed using the same two denaturation conditions used in the HCT116/DKO experiments (denaturation in the presence of no formamide and in the presence of 0.5% formamide) and identified both methylation-positive and methylation-negative common regions shared between individual tumor samples (FIGS. 16A and B, and Tables 6 and 7).

TABLE 6
Methylation-positive regions among tumor samples designated R123,
R147, R160, and R162.

| R123 positive | R147 positive | R160 positive | R162 positive |
|---|---|---|---|
| ACR | ADCY3 | ACR | ACR |
| AFAP1 | ADCY5 | ADCY3 | ACSL1 |
| ALPK3 | ADCY6 | ADCY6 | ADCY6 |
| ANKRD43 | ADRA1D | AFAP1 | ADRA2A |
| B3GALT6 | AFF3 | AFF3 | AFAP1 |
| BCL2L11 | AJAP1 | AJAP1 | AFF3 |
| C10orf72 | AKT1 | AKT1 | AJAP1 |
| C14orf2 | AMFR | ANKRD12 | ALDH1A3 |
| C20orf177 | ANAPC11 | APCDD1 | ALPK3 |
| CPT1B | ANKH | AQP5 | ALX3 |
| CPXM2 | ANKS6 | ARHGDIA | AMFR |
| CYP3A5 | APCDD1 | ARHGEF7 | AMH |
| DACT3 | AQP12A | ATP1A3 | AMY1B |
| DAZAP1 | AQP5 | ATP1B3 | ANKRD13D |
| DMRTA2 | ARHGDIA | B3GALT6 | APCDD1 |
| DRD4 | ARHGEF7 | BAG3 | ARHGDIA |
| FAM83F | ASXL1 | BAI2 | ASXL1 |
| FASTK | ATP1B3 | BCL2 | ATP1A3 |
| FBXL11 | AXIN2 | BCR | ATP1B3 |
| GP1BB | B3GALT6 | BRD7 | ATP5I |
| GPR17 | B3GNT1 | BRI3 | ATXN7 |
| GRIN2D | B4GALNT3 | BRUNOL4 | AXIN2 |
| GRWD1 | BAI2 | BTBD14A | B3GALT6 |
| H1FNT | BCL2 | C11orf80 | B4GALNT3 |
| HIC1 | BRD7 | C14orf2 | B4GALT4 |
| HNRPCL1 | BRF1 | C1orf34 | BAI2 |
| IFT140 | BRI3 | C1QTNF4 | BRUNOL4 |
| KCNH2 | BRUNOL4 | C20orf177 | C11orf9 |
| KCTD21 | BRUNOL5 | C6orf146 | C14orf2 |
| LOC164714 | BSN | C7orf41 | C1orf34 |
| LOC374569 | BTBD14A | CABP7 | C1orf69 |
| MECR | BTBD6 | CACNA1B | C1QTNF4 |
| MLC1 | C14orf2 | CAMK2B | C20orf118 |
| MOV10L1 | C16orf24 | CASQ2 | C20orf177 |
| NFIC | C16orf65 | CBFA2T3 | C2orf49 |
| NR2F1 | C16orf79 | CCDC40 | C6orf146 |
| OBSCN | C19orf26 | CDC34 | C6orf201 |
| PDE9A | C1orf34 | CDC42BPB | CABP7 |
| PDLIM4 | C1QTNF4 | CDH22 | CADPS |
| PHOX2B | C6orf146 | CDK5R1 | CALM2 |
| PRDM8 | C6orf201 | CDYL | CAMK2G |
| RHBDL3 | C7orf41 | CELSR1 | CDC34 |
| SCT | C9orf30 | CELSR2 | CDH22 |
| SDF4 | C9orf91 | CHD3 | CDK5R1 |
| SLC10A4 | CABP7 | CHD5 | CDV3 |
| SLC7A5 | CACNA1B | CHD6 | CDYL |
| SOX1 | CACNG7 | CIC | CELSR2 |
| SOX9 | CAMK2B | CLMN | CENTG2 |
| TBX4 | CAMK2G | CLPTM1L | CFC1 |
| TPPP3 | CAMK2N1 | COBL | CHD6 |
| TRPV4 | CAMK2N2 | COL18A1 | CHRD |
| WDR24 | CBFA2T3 | COLEC12 | CHSY1 |
| ZAR1 | CBX2 | CPT1B | CIC |
| ZFP36L2 | CCDC40 | CPXM2 | CLDN9 |
| ZNF524 | CCM2 | CRAMP1L | CLPTM1L |
| | | CRIP2 | COL4A1 |
| | | CRMP1 | CORO2B |
| | | CSK | CPT1B |
| | | CSNK1G2 | CPXM2 |
| | | CTNNBIP1 | CRAMP1L |
| | | CTSZ | CRMP1 |
| | | CUL3 | CRYBA2 |
| | | DACT3 | CSK |
| | | DDT | CTNNBIP1 |
| | | DDTL | CTNND2 |
| | | DIO3 | CUL3 |
| | | DKFZP564J102 | CUL4A |
| | | DMRTB1 | CYP26B1 |
| | | DNAJC5 | CYP3A5 |
| | | DTNB | DACT3 |
| | | DUSP22 | DAZAP1 |
| | | DVL3 | DDEF2 |
| | | ECOP | DMRTB1 |
| | | EML2 | DNAJA5 |
| | | EN2 | DNAJC5 |
| | | FAM53B | DNMT3A |
| | | FAM83F | DOCK5 |
| | | FBXL11 | DPP10 |
| | | FBXL16 | DTNB |
| | | FGFR3 | E2F5 |
| | | FGFRL1 | ECOP |
| | | FOXC1 | EPB49 |
| | | GNA12 | EPHA8 |
| | | GP1BB | EPHB2 |
| | | GPS1 | EPPK1 |
| | | GPT | FAM102B |
| | | GRB10 | FAM44A |
| | | GRWD1 | FAM49A |
| | | H1FNT | FAM59A |
| | | HIC1 | FAM83F |
| | | HOXA13 | FAM83H |
| | | HS6ST1 | FBXL16 |
| | | HS6ST3 | FDXR |
| | | HTR7 | FEV |
| | | IFT140 | FGFR3 |
| | | INHBB | FGFRL1 |
| | | ITPK1 | FLJ37440 |
| | | KCNH2 | FOSL2 |
| | | KCNIP4 | FOXC1 |
| | | KCNK3 | FOXK1 |
| | | KCTD21 | GAB2 |
| | | KIAA0664 | GALNT10 |
| | | KIAA0746 | GATA6 |
| | | KIAA1026 | GLCCI1 |
| | | KIF26A | GP1BB |
| | | LOC116236 | GPR12 |
| | | LOC164714 | GPRIN2 |
| | | LOC389813 | GPT |
| | | LOC91461 | GRIFIN |
| | | LPHN1 | GRIN2C |
| | | LRG1 | GRWD1 |
| | | LRRC4 | HIC1 |
| | | LRRC56 | HIST2H3C |
| | | LSDP5 | HOMER3 |
| | | LZTS2 | HOXA11 |
| | | MAN1C1 | HOXA13 |
| | | MAP3K3 | HS6ST1 |
| | | MAP4K2 | HS6ST3 |
| | | MAPK8IP2 | HTRA3 |
| | | MED16 | IGF2BP2 |
| | | MEIS3 | IGF2R |
| | | METRN | INHBB |
| | | MGAT4B | INSM1 |
| | | MLLT6 | IRX2 |
| | | MPP6 | ITPK1 |
| | | MUC1 | JAZF1 |
| | | MYRIP | JSRP1 |
| | | NBL1 | JUND |
| | | NFE2L3 | KCNB1 |
| | | NFIC | KCNF1 |
| | | NLRP5 | KCNH2 |
| | | NPAS3 | KCNJ14 |
| | | NPTXR | KCNK3 |
| | | NR2F1 | KCNK7 |
| | | NR2F6 | KIAA0664 |
| | | NR4A3 | KIAA0746 |
| | | OBSCN | KIAA1026 |
| | | ODC1 | KIAA1045 |
| | | ONECUT2 | KIAA1450 |
| | | PATZ1 | KIAA1618 |
| | | PDE10A | KIAA1641 |
| | | PHF21B | KL |
| | | PHLPP | KLF11 |
| | | PHOX2B | KLF2 |
| | | PHPT1 | LINGO1 |
| | | PIP4K2A | LOC164714 |
| | | PITPNM3 | LOC339123 |
| | | PPP1R12C | LOC374569 |
| | | PQLC3 | LOC389813 |
| | | PRDM8 | LOC653275 |
| | | PRKACG | LOC91461 |
| | | PRR6 | LPHN1 |
| | | PTCH1 | LRIG2 |
| | | PTPRN2 | LRP3 |
| | | RAB11FIP3 | LRRC14 |
| | | RAB11FIP4 | LYL1 |
| | | RAB11FIP5 | LZTS2 |
| | | RAB12 | MAP3K3 |
| | | RAB40C | MAP4K2 |
| | | RAD52 | MAPK11 |
| | | RANBP9 | MAPK8IP2 |
| | | RASSF8 | METRN |
| | | RBM38 | MFSD3 |
| | | RBPJ | MFSD7 |
| | | RERE | MLC1 |
| | | RFNG | MMP17 |
| | | RHBDL3 | MOV10L1 |
| | | SAMD4B | MPP6 |
| | | SCARF2 | MUC1 |
| | | SDF4 | NBL1 |
| | | SF1 | NCK2 |
| | | SH2B2 | NCOA2 |
| | | SH3PXD2B | NFE2L3 |
| | | SIX3 | NFIC |
| | | SLC24A3 | NFKB1 |
| | | SLC9A3R2 | NOPE |
| | | SMARCD3 | NPAS3 |
| | | SNCAIP | NPTXR |
| | | SNIP | NR2F6 |
| | | SORCS2 | NXPH4 |
| | | SOX1 | OBSCN |
| | | SOX9 | ODC1 |
| | | SPTBN4 | ONECUT2 |
| | | SSH1 | OTUD4 |
| | | STK11 | PAQR4 |
| | | SULF2 | PARD6G |
| | | TBX2 | PCSK6 |
| | | TCERG1L | PDE10A |
| | | TCF7 | PGF |
| | | TEX2 | PHLPP |
| | | TFAP2E | PHOX2B |
| | | THPO | PHPT1 |
| | | TMEM121 | PIP4K2A |
| | | TMEM16A | PITPNM3 |
| | | TMEM8 | PODXL2 |
| | | TNRC6B | PPP1R12C |
| | | TOX2 | PPP1R3D |
| | | TPPP3 | PRDM8 |
| | | TRIM28 | PRKACG |
| | | TRPV4 | PRKAG2 |
| | | TSPAN14 | PTCH1 |
| | | TTC7A | PWWP2 |
| | | TXNDC5 | QKI |
| | | UBE2F | RAB11FIP3 |
| | | UBE2Q1 | RAB11FIP4 |
| | | UBE2S | RAB11FIP5 |
| | | USP31 | RAB26 |
| | | WDR24 | RAE1 |
| | | WDR85 | RANBP9 |
| | | WSCD1 | RBM38 |
| | | ZAR1 | RBPMS2 |
| | | ZDHHC14 | RERE |
| | | ZFP36L2 | RGMA |
| | | ZMYND19 | RHBDL3 |
| | | ZNF282 | RHOQ |
| | | ZNF395 | RNF19B |
| | | ZNF524 | RORC |
| | | ZNF562 | RYK |
| | | ZNF592 | SDF4 |
| | | ZNRF1 | SDK1 |
| | | | SF1 |
| | | | SH2B2 |
| | | | SH3BP4 |
| | | | SIX3 |
| | | | SLC15A3 |
| | | | SLC24A3 |
| | | | SLC39A13 |
| | | | SLC9A3R2 |
| | | | SMARCD3 |
| | | | SMYD2 |
| | | | SNIP |
| | | | SOX1 |
| | | | SOX18 |
| | | | SP5 |
| | | | SPTBN2 |
| | | | SPTBN4 |
| | | | SRM |
| | | | SS18L1 |
| | | | STAU2 |
| | | | STIP1 |
| | | | STXBP5 |
| | | | SUMO3 |
| | | | TBX2 |
| | | | TBX4 |
| | | | TCBA1 |
| | | | TCEA2 |
| | | | TCF7 |
| | | | TEAD4 |
| | | | TENC1 |
| | | | TEX2 |
| | | | TFAP2E |
| | | | TFDP1 |
| | | | TGFBRAP1 |
| | | | THPO |
| | | | TMEM132D |
| | | | TMEM8 |
| | | | TMEPAI |
| | | | TPPP3 |
| | | | TRIO |
| | | | TSHZ1 |
| | | | TSPAN14 |
| | | | TSPAN33 |
| | | | TTC7A |
| | | | TTL |
| | | | TTYH3 |
| | | | TWIST1 |
| | | | TXNDC5 |
| | | | UBE2F |
| | | | UBE2I |
| | | | UBE2S |
| | | | UNCX |
| | | | VEGFC |
| | | | WDR24 |
| | | | WDR85 |
| | | | WTIP |
| | | | XKR6 |
| | | | XYLT1 |
| | | | YIF1A |
| | | | ZBTB39 |
| | | | ZBTB8 |
| | | | ZDHHC14 |
| | | | ZFP161 |
| | | | ZFP36L2 |
| | | | ZMYND19 |
| | | | ZNF2 |
| | | | ZNF395 |
| | | | ZNF524 |
| | | | ZNF660 |
| | | | ZNF710 |
| | | | ZNRF2 |

TABLE 7
Methylation-negative regions among tumor samples
designated R123, R147, R160, and R162.

| R123 | R147 | R160 | R162 |
|---|---|---|---|
| negative | negative | negative | negative |
| ADCY9 | ABCA7 | ABBA-1 | ADARB1 |
| ADRA2C | ADCY9 | ABCA7 | AGA |
| ARID1B | AGA | ADARB1 | ANKRD9 |
| B4GALNT4 | ATP10A | ADRBK1 | AQP12B |
| C11orf75 | B4GALNT4 | AGA | B4GALNT4 |
| CACNA1H | C11orf9 | AQP12A | BARX1 |
| CD81 | C3orf32 | AQP12B | BRD3 |
| CDC42BPB | C9orf72 | ARID1B | C10orf38 |
| CLMN | C9orf86 | B4GALNT4 | C10orf72 |
| CLN8 | CCDC42 | BARX1 | C3orf32 |
| COG1 | CD81 | BRD3 | C9orf30 |
| CTDSPL | COG1 | C10orf38 | C9orf37 |
| DDT | DUB3 | C6orf124 | C9orf61 |
| DDTL | DUSP22 | C9orf61 | C9orf86 |
| DGKD | ECHDC3 | C9orf72 | CACNA1H |
| DIP2C | EXOC3 | C9orf86 | CAMTA1 |
| EXOC3 | GPR137B | CCDC42 | CCDC42 |
| FGD5 | KIAA0467 | CD81 | CDRT15 |
| GSTT2 | KIR2DL3 | CDYL2 | CDYL2 |
| GTF2A1 | KIR2DS4 | CLN8 | CHD3 |
| JDP2 | KIR3DL1 | CSNK1E | CLMN |
| KIF13A | KIR3DL2 | CTDSPL | CLPTM1 |
| KRCC1 | KRTAP5-7 | DIP2C | CMTM4 |
| LOC116349 | LOC116349 | DVL1 | CTDSPL |
| MFRP | LOC441956 | DYRK1A | CTGLF1 |
| MMP24 | LRP5 | ECHDC3 | CTGLF4 |
| NKX6-2 | MEX3C | EFCBP2 | DDT |
| PAPPA | NKX6-2 | EPSTI1 | DDTL |
| PCSK6 | PER1 | FBXL16 | DIO3 |
| PLXNA1 | PLXND1 | FNDC5 | DIP2C |
| PLXND1 | RBM38 | FREQ | DUB3 |
| PXDN | REV1 | HBA2 | EEF1D |
| RBM38 | REXO1L1 | HBM | EFNA2 |
| SNN | SERPINF2 | HECA | EGFL7 |
| SPTBN2 | TP53TG3 | HEY1 | EXOC3 |
| SS18L1 | TTLL10 | HIST2H2AA3 | FAM108C1 |
| TMUB1 | TUBGCP5 | HIST2H2AA4 | FAM75B |
| UBXD8 | UTF1 | HIST2H3C | FAM78A |
| ZADH2 | WDR1 | IL17D | FAM81A |
| ZCCHC14 | ZDHHC11 | IRF2BP2 | FREQ |
| ZFP37 | ZFP28 | JPH3 | FTCD |
| ZNF419 | ZFP37 | KHDRBS3 | GSTT2 |
| | ZNF195 | KIAA0649 | HS3ST4 |
| | | KIAA0692 | ITPK1 |
| | | KIR2DL3 | JPH3 |
| | | KIR2DS4 | KCNC3 |
| | | KIR3DL1 | KHDRBS3 |
| | | KIR3DL2 | KIAA0467 |
| | | KIR3DP1 | KIAA0649 |
| | | KRTAP5-7 | KIF26A |
| | | LOC338328 | KIF7 |
| | | LOC392982 | KIR2DL3 |
| | | LOC440348 | KIR2DS4 |
| | | LOC440350 | KIR3DL1 |
| | | LOC441956 | KIR3DL2 |
| | | LOC653499 | KIR3DP1 |
| | | LRIG2 | KRTAP5-7 |
| | | MEX3C | LARGE |
| | | MGC21874 | LOC116349 |
| | | MUC20 | LOC338328 |
| | | NOMO1 | LOC441956 |
| | | NOPE | M-RIP |
| | | OR2A4 | MAPK6 |
| | | OR2A7 | MED16 |
| | | PCNX | METTL5 |
| | | PCSK6 | MLLT6 |
| | | PDS5B | MRPS6 |
| | | PLXND1 | NEK6 |
| | | POGZ | NFIL3 |
| | | POU4F1 | NFYB |
| | | PRDM15 | NOMO1 |
| | | PXDN | OR2A7 |
| | | QKI | PAPPA |
| | | RAP2A | PCNX |
| | | RBM38 | PHF2 |
| | | RCC2 | POGZ |
| | | REXO1L1 | PXDN |
| | | SNF1LK | RAB11FIP4 |
| | | SPTBN2 | RAP2A |
| | | SYT7 | RAPGEF1 |
| | | TBC1D3 | RBM38 |
| | | TBC1D3B | RNF130 |
| | | TBC1D3C | RNPEPL1 |
| | | TBC1D3G | RPS6KA5 |
| | | TBL1XR1 | RTN4R |
| | | TCEB3C | RXRA |
| | | TCEB3CL | SAMD4B |
| | | TP53TG3 | SH3PXD2B |
| | | USP22 | SHC2 |
| | | USP6 | SNF1LK |
| | | USP7 | SOHLH1 |
| | | UTF1 | SOLH |
| | | VEGFB | TBC1D3 |
| | | WNT3A | TBC1D3B |
| | | WNT4 | TBC1D3C |
| | | ZFP161 | TBC1D3G |
| | | ZFP37 | TBC1D9B |
| | | ZNF195 | TCEB3C |
| | | ZNF419 | TCEB3CL |
| | | | TCL6 |
| | | | TMEPAI |
| | | | TRAF3 |
| | | | UBE2E3 |
| | | | USF2 |
| | | | USP22 |
| | | | USP7 |
| | | | WSCD1 |
| | | | ZDHHC8 |
| | | | ZNF195 |
| | | | ZNF480 |
| | | | ZNRF3 |

Interestingly, among the loci identified were members of the Notch-Hes and Sonic hedgehog (Shh) pathways, two pathways implicated in the pathogenesis of medulloblastoma. Of the methylation-positive loci shared among all four patient samples, PRDM8, a putative negative regulator of the Notch-Hes pathway30 and HIC1, a putative tumor suppressor and negative regulator of the Shh pathway (Briggs et al., Genes & Development 22:770-785 (2008)) that is found to be frequently hypermethylated in medulloblastoma (Rood et al., Cancer Research 62:3794-3797 (2002)) were identified. In addition, in three of the four patient samples PTCH1, a negative regulator of the Shh pathway was found to be methylation-positive. Recently, PTCH1 mRNA expression was found to be absent with concomitant Shh pathway activation in a subset of medulloblastoma patient samples, and bisulfite sequence analysis of the PTCH1-1B promoter region failed to show hypermethylation (Pritchard & Olson, Cancer Genetics and Cytogenetics 180:47-50 (2008)). Interestingly, the methylation-positive signal mapped to the PTCH1-1C promoter region which was not evaluated in the previous study. When bisulfite sequence analysis was performed on this region in one of the tumors, the medulloblastoma sample was heavily methylated compared to the normal cerebellum control. Thus, differential denaturation under the conditions defined herein can identify cancer-specific common regions of differential CpG methylation in primary patient samples.

The previous examples are provided to illustrate but not limit the scope of the claimed inventions. Other variations of the disclosure will be readily apparent to those of ordinary skill in the art and encompassed by the following claims. All publications, patents and patent applications and other references cited herein are hereby incorporated by reference.

What is claimed is:

1. A method for identifying genomic DNA comprising a methylated DNA and a DNA palindrome, comprising the steps of:
a) isolating genomic DNA comprising the DNA palindrome and the methylated DNA;
b) fragmenting the genomic DNA;
c) denaturing unmethylated genomic DNA;
d) rehybridizing the denatured unmethylated DNA under suitable conditions for the DNA palindrome to form a snap back DNA;
e) digesting the rehybridized DNA with a nuclease that digests single strand DNA; and,
f) identifying the genomic DNA comprising the methylated DNA and the snap back DNA comprising the DNA palindrome.

2. The method according to claim 1, wherein the method further comprises identifying regions of the genomic DNA comprising the methylated DNA and the DNA palindrome by hybridization of the genomic DNA fragments with a human genomic DNA array.

3. The method according to claim 2, wherein the method further comprises the steps of:
a) isolating genomic DNA comprising the DNA palindrome or the methylated DNA from a population of cells;
b) denaturing the isolated, unmethylated DNA;
c) rehybridizing the denatured isolated DNA under suitable conditions for the DNA palindrome to form a snap back DNA and to keep the methylated DNA hybridized;
d) digesting the rehybridized DNA with a nuclease that digests single strand DNA to form double stranded DNA fragments comprising the snap back DNA and the methylated DNA;
e) digesting the double stranded DNA fragments comprising the snap back DNA with a nucleotide sequence specific restriction enzyme;
f) adding a sequence specific linker nucleotide sequence to one end of each stand of the double strand DNA comprising the snap back DNA;
g) amplifying the DNA fragments comprising the added linker using a labeled linker sequence specific primer corresponding to the sequence specific linker added in step (f); and,
h) hybridizing the methylated DNA and the amplified DNA fragments comprising the snap back DNA to a genomic DNA library and identifying the genomic DNA region comprising the palindrome or the methylated DNA.

4. The method according to claim 3, wherein the amplified DNA fragments comprising the snap back DNA are mixed and co-hybridized in step (h) with a sample of high molecular weight DNA from a normal cell population that has been digested with S1 nuclease, and the restriction enzyme of step (e), adding a linker labeled with a second single label, and amplified.

5. The method according to claim 3, wherein the single strand nuclease comprises S1 nuclease.

6. The method according to claim 3, wherein the restriction enzyme comprises MspI, TaqI, or MseI.

7. The method according to claim 3, wherein the genomic DNA is fragmented by a chemical, physical, or enzymatic method.

8. A method for classifying a population of cancer cells, comprising the steps of:
a) identifying regions of genomic DNA comprising a methylated DNA and a snap back DNA comprising a DNA palindrome; and,
b) using the identity of genomic DNA regions comprising the palindromes or methylated DNA to classify the population of cancer cells.

9. The method according to claim 8, wherein step (b) further comprises fragmenting the genomic DNA; denaturing the unmethylated genomic DNA fragments; incubating the denatured and unmethylated genomic DNA fragments under conditions conducive to the formation of snap back DNA by genomic DNA fragments comprising the DNA palindrome; and identifying regions of genomic DNA containing the DNA palindrome and the methylated DNA to form a profile.

10. The method of claim 9, further comprising comparing the profile of genomic DNA comprising a DNA palindrome and methylated DNA of the cancer cell population to a population of normal cells.

11. A method for detecting a population of cancer cells, comprising the steps of:
a) isolating genomic DNA from a cell population;
b) identifying a plurality of genomic DNA regions comprising methylated DNA and snap back DNA comprising a palindrome; and,
c) using the identity of the plurality of genomic DNA regions comprising the methylated DNA and palindrome to detect the population of cancer cells.

12. The method according to claim 11, wherein the method further comprises fragmenting the isolated genomic DNA; denaturing the unmethylated genomic DNA fragments; incubating the denatured and unmethylated genomic DNA fragments under conditions conducive to formation of snap back DNA comprising the DNA palindrome; digesting denatured, single strand DNA; and identifying a plurality of regions of the genomic DNA containing the DNA palindrome and the methylated DNA to form a profile.

13. The method of claim 12, further comprising comparing the profile of the cancer cell population to a population of normal cells, wherein the cancer cell population comprises genomic DNA comprising the DNA palindrome and the methylated DNA.

14. A method for determining a region of genomic DNA that comprises an unmethylated CpG island, comprising:
a) digesting genomic DNA with a methylation sensitive restriction enzyme;
b) amplifying the DNA fragments using a labeled linker sequence; and,
c) hybridizing the amplified DNA fragments to a genomic DNA library and identifying the genomic DNA region comprising the palindrome.

15. A method for identifying a region of genomic DNA comprising a DNA palindrome, comprising the steps of:
a) isolating genomic DNA comprising the DNA palindrome or the methylated DNA from a population of cells;
b) denaturing the isolated, unmethylated DNA;
c) incubating denatured isolated DNA under conditions conducive to inducing formation of a snap back DNA rather than inter-molecular hybridization, the snap back DNA comprising the DNA palindrome;
d) digesting the denatured, unmethylated DNA;
e) isolating the methylated DNA and the snap back DNA;
f) denaturing the methylated DNA and the snap back DNA;
g) incubating the methylated DNA and the snap back DNA under conditions conducive to inducing formation of the snap back DNA;
h) digesting the denatured methylated DNA; and,
i) identifying one or more regions of the genomic DNA comprising the snap back DNA thereby identifying one or more regions of the genomic DNA comprising the DNA palindrome.

16. The method of claim 15, wherein denaturation of methylated DNA comprises alkaline denaturation or heating and an agent capable of lowering the melting temperature of methylated DNA.

17. The method claim 16, wherein the agent comprises formamide.

18. A method for isolating genomic DNA comprising a methylated DNA, comprising the steps of:
a) incubating isolated genomic DNA under conditions conducive to hybridization of the methylated DNA and to denaturation of an unmethylated DNA;
b) digesting the unmethylated DNA; and,
c) isolating the genomic DNA comprising methylated DNA.

19. The method of claim 18, further comprising identifying regions of the genomic DNA comprising methylated DNA.

20. The method of claim 18, further comprising additional steps between steps (a) and (b) comprising,
incubating the isolated genomic DNA under conditions conducive to inducing formation of a snap back DNA rather than inter-molecular hybridization, wherein the unmethylated DNA comprises a DNA palindrome capable of forming snap back DNA;
isolating the methylated DNA and the unmethylated DNA comprising the DNA palindrome; and,
denaturing the unmethylated DNA comprising the DNA palindrome.

21. The method of claim 18, wherein the conditions in step (a) used to denature unmethylated DNA comprise a temperature and a concentration of formamide conducive to allowing for digestion of the unmethylated DNA in step (b).

22. The method of claim 18, wherein the denatured, unmethylated DNA is digested with a single strand nuclease.

23. A method for identifying CpG densities and degrees of CpG methylation in one or more regions of genomic DNA, comprising the steps of:
a) isolating genomic DNA;
b) denaturing the isolated, unmethylated DNA;
c) digesting the unmethylated DNA;
d) isolating the genomic DNA comprising methylated DNA; and,
e) enriching for regions of genomic DNA having a specific CpG density and degree of CpG methylation.

24. The method of claim 23, wherein step (e) further comprises the steps of:
denaturing the genomic methylated DNA under a temperature, a concentration of formamide, and a concentration of NaCl tuned for hybridization of one or more regions of genomic DNA having a specific CpG density and degree of CpG methylation;
digesting the denatured genomic methylated DNA; and,
identifying the undigested regions of genomic DNA comprising methylated DNA.


SEQUENCE LISTING (52 sequences)

SEQ ID NO: 1 - DNA, 25 bp, Artificial
  ligation-mediated PCR (LM-PCR) synthetic oligonucleotide JW102g
gcggtgacccgggagatctgaattg

SEQ ID NO: 2 - DNA, 18 bp, Artificial
  ligation-mediated PCR (LM-PCR) synthetic oligonucleotide JW103pc2
cgcaattcagatctcccg

SEQ ID NO: 3 - DNA, 25 bp, Artificial
  ligation-mediated PCR (LM-PCR) synthetic oligonucleotide JW102
gcggtgacccgggagatctgaattc

SEQ ID NO: 4 - DNA, 18 bp, Artificial
  ligation-mediated PCR (LM-PCR) synthetic oligonucleotide JW103p2
cggaattcagatctcccg

SEQ ID NO: 5 - DNA, 18 bp, Artificial
  ligation-mediated PCR (LM-PCR) synthetic oligonucleotide JW103pcTA
tacaattcagatctcccg

SEQ ID NO: 6 - DNA, 28 bp, Artificial
  microarray PCR synthetic linker specific primer JW102gMSP, MspI primer
gcggtgacccgggagatctgaattgcgg

SEQ ID NO: 7 - DNA, 28 bp, Artificial
  microarray PCR synthetic linker specific primer JW102Taq
gcggtgacccgggagatctgaattccga

SEQ ID NO: 8 - DNA, 28 bp, Artificial
  microarray PCR synthetic linker specific primer JW102gMse
gcggtgacccgggagatctgaattgtaa

SEQ ID NO: 9 - DNA, 23 bp, Artificial
  PCR synthetic oligo ECM15154
acacctttcacacctcgcttctc

SEQ ID NO: 10 - DNA, 26 bp, Artificial
  PCR synthetic oligo ECM15851
ggcagataaagaagagacagtggttg

SEQ ID NO: 11 - DNA, 26 bp, Artificial
  nucleotide primer
ctcctcagtcccctatgactacattt

SEQ ID NO: 12 - DNA, 26 bp, Artificial
  nucleotide primer
gcccagccaatatacaactgtaaagc

SEQ ID NO: 13 - DNA, 20 bp, Artificial
  nucleotide primer
gtctagggctcctgctcctt

SEQ ID NO: 14 - DNA, 20 bp, Artificial
  nucleotide primer
gcaggagctttggaattacg

SEQ ID NO: 15 - DNA, 19 bp, Artificial
  nucleotide primer
ccggagggaagcctagagt

SEQ ID NO: 16 - DNA, 21 bp, Artificial
  nucleotide primer
ggctgttccagtacatcctca

SEQ ID NO: 17 - DNA, 20 bp, Artificial
  nucleotide primer
gcagacactcctgacagctc

SEQ ID NO: 18 - DNA, 19 bp, Artificial
  nucleotide primer
cggtcttagtccgacttcc

SEQ ID NO: 19 - DNA, 20 bp, Artificial
  nucleotide primer
agcccattcattcctccatt

SEQ ID NO: 20 - DNA, 21 bp, Artificial
  nucleotide primer
cgcttttatcaggtaggcagt

SEQ ID NO: 21 - DNA, 19 bp, Artificial
  nucleotide primer
cacgactccaccaccatgt

SEQ ID NO: 22 - DNA, 19 bp, Artificial
  nucleotide primer
ctacgctgcagcaagaagc

SEQ ID NO: 23 - DNA, 18 bp, Artificial
  nucleotide primer
agcccgatctgggttctt

SEQ ID NO: 24 - DNA, 18 bp, Artificial
  nucleotide primer
gagaaccaccgccgtcac

SEQ ID NO: 25 - DNA, 20 bp, Artificial
  nucleotide primer
tgcataaacaacctcggaaa

SEQ ID NO: 26 - DNA, 18 bp, Artificial
  nucleotide primer
gcagacccgtggacagac

SEQ ID NO: 27 - DNA, 20 bp, Artificial
  nucleotide primer
aagaaggactcgctgctcac

SEQ ID NO: 28 - DNA, 20 bp, Artificial
  nucleotide primer
ctccagtggcaaagcctaag

SEQ ID NO: 29 - DNA, 18 bp, Artificial
  nucleotide primer
gagggaaggggcagagtt

SEQ ID NO: 30 - DNA, 23 bp, Artificial
  nucleotide primer
tgacagctcctgtatgtcagaga

SEQ ID NO: 31 - DNA, 20 bp, Artificial
  nucleotide primer
gaagcagggggagaaagaag

SEQ ID NO: 32 - DNA, 19 bp, Artificial
  nucleotide primer
gctcttccaggctcaaagg

SEQ ID NO: 33 - DNA, 20 bp, Artificial
  nucleotide primer
ctgcaccttcacattttcca

SEQ ID NO: 34 - DNA, 20 bp, Artificial
  nucleotide primer
ttcttgtgctgtgtggacct

SEQ ID NO: 35 - DNA, 21 bp, Artificial
  nucleotide primer
caaacaccagtagttcgttcg

SEQ ID NO: 36 - DNA, 20 bp, Artificial
  nucleotide primr
cctttggctcagcaattctc

SEQ ID NO: 37 - DNA, 20 bp, Artificial
  nucleotide primer
cctagttgcccctagtggtg

SEQ ID NO: 38 - DNA, 20 bp, Artificial
  nucleotide primer
gttctggggtcaagttccaa

SEQ ID NO: 39 - DNA, 18 bp, Artificial
  nucleotide primer
aacctccacgtggccttc

SEQ ID NO: 40 - DNA, 21 bp, Artificial
  nucleotide primer
cctcacaacatgaggaagtgg

SEQ ID NO: 41 - DNA, 21 bp, Artificial
  nucleotide primer
gcacaggtattttgcagttcg

SEQ ID NO: 42 - DNA, 20 bp, Artificial
  nucleotide primer
gcgaagtaccaggacaaacc

SEQ ID NO: 43 - DNA, 23 bp, Artificial
  nucleotide primer
aatttaatggagatgaagaatgg

SEQ ID NO: 44 - DNA, 20 bp, Artificial
  nucleotide primer
tcaaactcccaaaaaaaaca

SEQ ID NO: 45 - DNA, 20 bp, Artificial
  nucleotide primer
tttttagtttaggttagggt

SEQ ID NO: 46 - DNA, 20 bp, Artificial
  nucleotide primer
acaccctttctaaataaaac

SEQ ID NO: 47 - DNA, 23 bp, Artificial
  nucleotide primer
atctcaatacatccattttctca

SEQ ID NO: 48 - DNA, 20 bp, Artificial
  nucleotide primer
gttgtatatggagattttgt

SEQ ID NO: 49 - DNA, 21 bp, Artificial
  nucleotide primer
agaaatttttttagggggagt

SEQ ID NO: 50 - DNA, 22 bp, Artificial
  nucleotide primer
acttactccaacctattaaaca

SEQ ID NO: 51 - DNA, 21 bp, Artificial
  nucleotide primer
gaggattgtagaagaatatta

SEQ ID NO: 52 - DNA, 20 bp, Artificial
  nucleotide primer
acatttaaataacatacccc